(19) United States
(12) Patent Application Publication
(10) Pub. No.: US 2009/0004171 A1
(43) Pub. Date: Jan. 1, 2009

(54) COMPOUND PROFILING METHOD

(75) Inventors: Masato Miyake, Koutou-ku (JP); Yoshiji Fujita, Koutou-ku (JP)

Correspondence Address: SEED INTELLECTUAL PROPERTY LAW GROUP PLLC, 701 FIFTH AVE, SUITE 5400, SEATTLE, WA, 98104, US

(73) Assignee: CYTOPATHFINDER, INC., Koutou-ku (JP)

(21) Appl. No.: 12/101,719

(22) Filed: Apr. 11, 2008

Related U.S. Application Data
(60) Provisional application No. 60/911,843, filed on Apr. 13, 2007.

(51) Int. Cl. A61K 38/44; C12Q 1/68; C40B 30/06; A61P 35/00; C12M 1/34

(52) U.S. Cl. 424/94.4; 435/6; 506/10; 435/287.2

(57) ABSTRACT

The present invention provides a method for deriving an upstream or downstream component of a component necessary for phenotypic alteration of a living organism, the method comprising the steps of: specifying a pathway of interest related to the phenotypic alteration and a reference pathway different from the pathway of interest, and specifying a stimulant of interest and a reference stimulant which respectively stimulate the pathway of interest and the reference pathway; giving the stimulant of interest to the living organism to identify a collection of components of interest necessary for the phenotypic alteration; giving the reference stimulant to the living organism to identify a collection of reference components necessary for the phenotypic alteration; calculating an intersection between the collections of the components of interest and the reference components; and calculating a differential collection by subtracting the intersection from the collection of components of interest.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 230032_{—}401_SEQUENCE_LISTING.txt. The text file is 331 KB, was created on Apr. 11, 2008, and is being submitted electronically via EFS-Web, concurrent with the filing of the specification.

### BACKGROUND OF THE INVENTION

1. Field of the Invention

The present invention is related to the technologies of classifying compounds. More specifically, the present invention is related to a method for profiling compounds based on biological functions and its relevant inventions.

2. Description of Background Art

Biological actions of compounds essential for our daily life such as pharmaceutical products, cosmetics, and food additives are not always fully understood. There are a number of such cases in which unexpected toxicity has developed into a social problem, or unexpected pharmacological effects have been discovered or the like. While the antiepilepsy drug “thalidomide” has developed into a drug induced social problem of cacomelia, it has also been shown to exhibit effects on Hansen's disease or myeloma, which is a typical example. As such, there are still demands in which compound profiling (or biological activity analysis) serves the development of use of compounds and securing safety, while the number of novel compounds to be on the market is decreasing.

Bowers, P. M. et al. (Use of logic relationships to decipher protein network organization. Science 306, 2246-2249 (2004)) proposed that gene occurrence in the genome is correlated with pathways, and thus it is possible to analyze pathways using relative genome methods. However, the present inventors have investigated this method using an animal cell, and it turned out that this method could not be used.

Dan, S. et al. (Cancer Res. 62; 1139-1147, (2002)) proposed a method for screening a drug having similar biological activity, from similarities in growth inhibitory effects or gene expression patterns upon addition of a drug to a variety of cell species. This method does not allow derivation of a pathway relating to biological activity of a drug, which is to be achieved by the present invention. Therefore, this method cannot be used for target analysis of a drug or multidrug combination design of drugs.

Perlman, Z. E. et al. (Science 306; 1194-1198, (2004)) discloses a method for evaluating effects of a drug on a single cell by means of time-lapsed alteration pattern of a plurality of gene expression reporters. This method allows predicting the similarity of drugs, however, it cannot be used for target analysis of a drug or multidrug combination design of drugs.

### DISCLOSURE OF THE INVENTION

### Summary of Invention

### Problems to be Solved by the Invention

The present invention provide a novel method for clarifying and investigating an intracellular pathway, used for biological activity of a compound, as effective information in order to analyze utility or side effects (toxicity) of a compound on a biological entity to answer the above-mentioned demands from society.

### Means for Solving the Problem

Apoptosis of a human cell occurs in all cells from all tissues due to caspase activity. Further, when an animal cell grows, activities of Rb and E2F are necessary. As such, when the biological entities in the issue are the same, it is known that the same component is used for effecting the same cellular function. The present inventors have found a method for clarifying a pathway relating to a function of a cell in a tissue from the expression occurrence of the intracellular components necessary for the cellular function in a tissue by targeting cells from the same biological entity.

As such, the present invention provides the following:

Item 1 A method for deriving an upstream or downstream component of a component necessary for a phenotypic alteration of a living organism, the method comprising the steps of:
A) specifying a pathway of interest related to the phenotypic alteration and a reference pathway different from the pathway of interest, and specifying a stimulant of interest and a reference stimulant which respectively stimulates the pathway of interest and the reference pathway;
B) giving the stimulant of interest to the living organism to identify a collection of components of interest necessary for the phenotypic alteration;
C) giving the reference stimulant to the living organism to identify a collection of the reference components necessary for the phenotypic alteration;
D) calculating an intersection between the collection of the components of interest and the reference components; and
E) calculating a differential collection by subtracting the intersection from the collection of components of interest, wherein a component which belongs to the differential collection is determined to be present upstream or downstream of the intersection.

Item 2 The method according to item 1, wherein the living organism is a cell.

Item 3 The method according to item 1, wherein the living organism is grown under two or more different conditions.

Item 4 The method according to item 1, wherein the component is induced from functional assay data which is indicative of a cell, tissue or an individual.

Item 5 The method according to item 1, wherein the component is selected based on a functional assay from a limited number of candidate genes including miRNA.

Item 6 The method according to item 1, wherein the component is a target calculated based on functional assay data from the target collection of the stimulant.

Item 7 The method according to item 1, wherein the component is a protein, a nucleic acid or both, which has an effect on a phenotype of interest.

Item 8 The method according to item 1, wherein the component is derived from the result of a functional screening from a limited number of functional nucleic acid libraries.

Item 9 The method according to item 1, wherein the stimulant is an antibody, an RNA interference agent or a molecular target inhibitor.

Item 10 A method for profiling a compound comprising the step of repeatedly applying the method according to item 1.

Item 11 A process for profiling a compound which can be combined for use in achieving the phenotypic alteration, the process comprising the step of repeatedly applying the method according to item 1, wherein the process further comprises the steps of:
A) calculating the collection of components of interest which increases the phenotypic alteration expected by a living organism under a culture condition in which the compound is added;
B) calculating the collection of components of interest which increases the phenotypic alteration expected by a living organism under a culture condition in which there is no compound;
C) calculating a differential collection of the collection of components of interest and the collection of reference components thereby calculating a specific component collection appearing under the culture conditions with a compound added thereto;
D) calculating a common pathway of components included in the specific component collection; and
E) selecting a compound which targets the common pathway.

Item 12 A process for searching for a target of a compound, comprising the method according to item 1, the process further comprising the steps of:
A) calculating the collection of components of interest which increases the phenotypic alteration expected by a living organism under a culture condition in which the compound which can be combined for use in achieving the phenotypic alteration is added;
B) calculating the collection of components of interest which increases the phenotypic alteration expected by a living organism under a culture condition in which there is no compound which can be combined for use in achieving the phenotypic alteration;
C) calculating a differential collection of the collection of components of interest and the collection of reference components, thereby calculating a specific component collection appearing under the culture conditions with a compound added thereto;
D) calculating a common pathway of components included in the specific component collection; and
E) selecting a target included in the common pathway.

Item 13 A process for searching for a target of a similar compound, comprising the method according to item 1, wherein the process comprises the steps of:
A) calculating the collection of components of interest which increases the phenotypic alteration expected by a living organism in a culture circumstance in which the compound which can be combined for use in achieving the phenotypic alteration is added;
B) calculating the collection of components of interest which increases the phenotypic alteration expected by a living organism under a culture condition in which there is no compound which can be combined for use in achieving the phenotypic alteration;
C) calculating a differential collection of the collection of components of interest and the collection of reference components, thereby calculating a specific component collection appearing under the culture condition with a compound added thereto;
D) calculating a common pathway of components included in the specific component collection; and
E) selecting a target of a similar compound from the common pathway.

Item 14 A method for inhibiting breast cancer using a combination of DXR and at least an inhibitor of the EphA family.

Item 15 A method for inhibiting breast cancer using a combination of DXR and at least an inhibitor of the EphB family.

Item 16 A method for inhibiting breast cancer using a combination of DXR and at least an inhibitor of c-KIT.

Item 17 A method for inhibiting breast cancer using a combination of DXR and at least an inhibitor of ALK.

Item 18 A system for deriving an upstream or downstream component necessary for the phenotypic alteration of a living organism, the system comprising:
A) a computer for specifying a pathway of interest related to the phenotypic alteration and a reference pathway different from the pathway of interest, and specifying a stimulant of interest and a reference stimulant which respectively stimulate the pathway of interest and the reference pathway;
B) an assay system for giving the stimulant of interest to the living organism to identify a collection of components of interest necessary for the phenotypic alteration;
C) an assay system for giving the reference stimulant to the living organism to identify a collection of reference components necessary for the phenotypic alteration;
D) a computer for calculating an intersection between the collection of the components of interest and the reference components; and
E) a computer for calculating a differential collection by subtracting the intersection from the collection of components of interest, wherein a component which belongs to the differential collection is determined to be present upstream or downstream of the intersection.

Item 19 A program for implementation by a computer to conduct a method for deriving an upstream or downstream component necessary for the phenotypic alteration of a living organism, the method comprising the steps of:
A) specifying a pathway of interest related to the phenotypic alteration and a reference pathway different from the pathway of interest, and specifying a stimulant of interest and a reference stimulant which respectively stimulate the pathway of interest and the reference pathway;
B) giving the stimulant of interest to the living organism to identify a collection of components of interest necessary for the phenotypic alteration;
C) giving the reference stimulant to the living organism to identify a collection of reference components necessary for the phenotypic alteration;
D) calculating an intersection between the collection of the components of interest and the reference components; and
E) calculating a differential collection by subtracting the intersection from the collection of components of interest, wherein a component which belongs to the differential collection is determined to be present upstream or downstream of the intersection.

Item 20 A storage medium with a program stored thereon for an implementation by a computer to conduct a method for deriving upstream or downstream of a component necessary for phenotypic alteration of a living organism, the method comprising the steps of:
A) specifying a pathway of interest related to the phenotypic alteration and a reference pathway different from the pathway of interest, and specifying a stimulant of interest and a reference stimulant which respectively stimulates the pathway of interest and the reference pathway;
B) giving the stimulant of interest to the living organism to identify a collection of components of interest necessary for the phenotypic alteration;
C) giving the reference stimulant to the living organism to identify a collection of reference components necessary for the phenotypic alteration;
D) calculating an intersection between the collection of the components of interest and the reference components; and
E) calculating a differential collection by subtracting the intersection from the collection of components of interest, wherein a component which belongs to the differential collection is determined to be present upstream or downstream of the intersection.

Item 21 A composition for inhibiting breast cancer comprising a combination of DXR and at least an inhibitor of the EphA family.

Item 22 A composition for inhibiting breast cancer comprising a combination of DXR and at least an inhibitor of the EphB family.

Item 23 A composition for inhibiting breast cancer comprising a combination of DXR and at least an inhibitor of c-KIT.

Item 24 A composition for inhibiting breast cancer comprising a combination of DXR and at least an inhibitor of ALK.

Hereinafter, the present invention will be described by way of preferred embodiments. It will be understood by those skilled in the art that the embodiments of the present invention can be appropriately made or carried out based on the description of the present specification and the accompanying drawings, and commonly used techniques well known in the art. The function and effect of the present invention can be easily recognized by those skilled in the art.

### THE EFFECTS OF THE INVENTION

The present invention provides a method for profiling a compound, thereby allowing investigation of the compound with respect to its biological function in a biological entity.

### BRIEF DESCRIPTION OF THE DRAWINGS

FIG. 1 depicts an exemplary quantification algorithm of component occurrence relating to the nerve projection extension. The pathway components requiring retinoic acid (RA) triggered neurite outgrowth were elucidated from the hit siRNAs obtained by functional screening. Similarly, the pathway components requiring NGF triggered neurite outgrowth were elucidated from the hit siRNAs obtained by the functional screening. As the next step, the intersection subset of the components was obtained from these pathway component sets. The specific pathway components of RA triggered neurite outgrowth were elucidated by subtraction of the pathway components requiring RA triggered neurite outgrowth and the intersection subset. Similarly, the specific pathway components of NGF triggered neurite outgrowth were elucidated by subtraction of the pathway components requiring NGF triggered neurite outgrowth and the intersection subset. Then, all the known molecular relations among the components in each of the sets were added and then contradictory relation data was subtracted against the neurite outgrowth acceleration. Finally, all of the remaining molecular relations were integrated.

FIG. 2 depicts an exemplary molecular relationship extraction algorithm. The first step defines the endpoint molecules of the pathway. The second step is to elucidate all the known molecular relation data from each the hit component to the endpoint molecules. The third step is subtraction of contradictory relations against the phenotypic change. After this process for all the hit components, all the molecular relation data is superimposed and the overlapped molecular relations are removed. Then, the remaining components and the relations are obtained.

FIG. 3A depicts an exemplary of components and pathway relating to the projection extension by retinoic acid (RA). The output graph indicates molecular relations between the hit molecules (RAR, JAK1, and JAK3) and the endpoint molecules (ROR and RET).

FIG. 3B depicts an exemplary of components and pathway relating to the projection extension by Nerve Growth Factor (NGF). The output graph indicates molecular relations between the hit molecules (IRS, PDGFR, NTRK1, and RPHB2) and the endpoint molecules (ROR and RET).

FIG. 4 depicts an exemplary of components and pathway relating to the projection extension by retinoic acid (RA) and Nerve Growth Factor (NGF). The output graph indicates molecular relations between the hit molecules (RAR, JAK1, and JAK3) for retinoic acid (RA), the hit molecules (IRS, PDGFR, NTRK1, and RPHB2) for Nerve Growth Factor (NGF), and the endpoint molecules (ROR and RET).

FIG. 5A depicts an exemplary quantification algorithm of component occurrence relating to DXR sensitivity. To elucidate DXR-enhanced pathway candidates of SK-BR-3 cell line, the hit components were elucidated by subtraction of intersection subset of the hit components in the presence or the absence of DXR. Then, the molecular relations between the hit molecules elucidated above and the endpoint molecule (RB) defined were elucidated by using the algorithm described in FIG. 2. To elucidate DXR-suppressed pathway candidates of SK-BR-3 cell line, the hit components were elucidated by subtraction of the intersection subset of the hit components in the presence or the absence of DXR. Then, the molecular relations between the hit molecules elucidated above and the endpoint molecule (RB) defined were elucidated by using the algorithm described in FIG. 2. To elucidate central pathway candidates of breast cancer cell lines, the intersection subset was elucidated from the intersection subsets in the presence or the absence of DXR in SK-BR-3 and T47D culture conditions. Then, the molecular relations between the molecules in the intersection subset elucidated above and the endpoint molecule (RB) defined were elucidated by using the algorithm described in FIG. 2. DXR-resistant growth pathway candidates were elucidated from the hit molecules in MCF7, which is a DXR resistant cell line, by using the algorithm described in FIG. 2. All the pathway candidates were integrated.

FIG. 5B depicts elucidation of the central pathways extracted from an exemplary quantification algorithm of component occurrence relating to the DXR sensitivity shown in FIG. 5A.

FIG. 5C depicts elucidation of DXR-enhanced pathways extracted from an exemplary quantification algorithm of component occurrence relating to the DXR sensitivity shown in FIG. 5A.

FIG. 5D depicts elucidation of DXR-suppressed pathways extracted from an exemplary quantification algorithm of component occurrence relating to the DXR sensitivity shown in FIG. 5A.

FIG. 5E depicts elucidation of DXR-resistant growth pathways extracted from an exemplary quantification algorithm of component occurrence relating to the DXR sensitivity shown in FIG. 5A.

FIG. 5F depicts integrated data of an exemplary quantification algorithm of component occurrence relating to the DXR sensitivity shown in FIG. 5A.

FIG. 6 depicts an exemplary of extraction of common pathway for the DXR independent pathways in SK-BR-3. The common molecules FER, EPHB6 and TYK2 experimentally elucidated are connected to the defined endpoint RB through the molecular relations described in the graph. Shaded boxes indicate experimental hits for DXR sensitive cell line.

FIG. 7 depicts an exemplary of extraction of pathway which is inhibited by DXR, i.e. DXR-suppressed pathways in SK-BR-3. The molecules Tie-1, Tie-2, ERBB2, CSF-1, BLK, and BTK elucidated as the DXR-suppressed pathway components are connected to the defined endpoint RB through the molecular relations described in the graph. Shaded boxes indicate experimental hits for DXR sensitive cell line.

FIG. 8 depicts an exemplary of extraction of pathway which is increased by DXR, i.e. DXR-enhanced pathways in SK-BR-3. The molecules EPHB4, DDR1, EPHA3, EPHA4, and EPHA7 elucidated as the DXR-enhanced pathway components are connected to the defined endpoint RB through the molecular relations described in the graph. Shaded boxes indicate experimental hits for DXR sensitive cell line.

FIG. 9 depicts an exemplary extraction of growth of a cell having DXR resistance, i.e. DXR-resistant growth pathways in MCF7. The molecules C-KIT, and ALK elucidated as the DXR-resistant pathway components are connected to the defined endpoint RB through the molecular relations described in the graph.

FIG. 10 depicts an exemplary extraction of DXR-dependent and independent pathways in SK-BR-3. Black wide lines/arrows indicate the DXR-independent pathways. Shaded intermediate lines/arrows indicate the DXR-enhanced pathways. Black narrow lines/arrows indicate the DXR-suppressed pathways. As shown in FIG. 10, the present invention elucidated how known anti-cancer agents function in the cells. Therefore, Herceptin and XL647(PI) effects on ErbB2 resulting in a DXR-suppression. EphB6, VEFGR, Tyk2, EphA3, EphA4, and EphA7 turned out to be potential targets for screening anti-cancer agents (BBRC (2004) 318:882, Mol. Pharmacol. (2004) 66:635, Cancer & Metastasis 22, 423-434 (2003), Cytokine & Growth Factor Reviews (2004) 15:419). Recently, VEGFR has been clinically determined to be a DXR enhance target. Therefore, the present invention clearly demonstrates that it provides effective screening methods.

FIG. 11 depicts an exemplary example of the concept of the present invention, reactivity of compounds to cell species for testing and references of the cell species (such as cells 1, 2, 3, 4 and 5, which have larger to smaller similarities to the test cell species) are analyzed and component collections necessary for the biological activity of a compound are determined. Thereafter, components different from cell species to cell species are determined from upstream to downstream by the means of the present methods of the present invention and the component essential for the action of the component are determined.

FIG. 12 depicts an exemplary configuration of a computer 500 for executing the compound profiling method of the present invention.

### BRIEF DESCRIPTION OF SEQUENCE LISTING

SEQ ID NO: 1: nucleic acid sequence of Homo sapiens EPH receptor A1 (EPHA1), mRNA. ACCESSION NM_{—}005232

SEQ ID NO: 2: amino acid sequence of Homo sapiens EPH receptor A1 (EPHA1)

SEQ ID NO: 3: nucleic acid sequence of Homo sapiens EPH receptor A2 (EPHA2), mRNA. ACCESSION NM_{—}004431

SEQ ID NO: 4: amino acid sequence of Homo sapiens EPH receptor A2 (EPHA2), mRNA. ACCESSION NM_{—}004431

SEQ ID NO: 5: nucleic acid sequence of Homo sapiens EPH receptor A3 (EPHA3), transcript variant 1, mRNA. ACCESSION NM_{—}005233.

SEQ ID NO: 6: amino acid sequence of Homo sapiens EPH receptor A3 (EPHA3), transcript variant 1, mRNA. ACCESSION NM_{—}005233.

SEQ ID NO: 7: nucleic acid sequence of Homo sapiens EPH receptor A4 (EPHA4), mRNA. ACCESSION NM_{—}004438 XM_{—}379155

SEQ ID NO: 8: amino acid sequence of Homo sapiens EPH receptor A4 (EPHA4), mRNA. ACCESSION NM_{—}004438 XM_{—}379155

SEQ ID NO: 9: nucleic acid sequence of Homo sapiens EPH receptor A7 (EPHA7), mRNA. ACCESSION NM_{—}004440

SEQ ID NO: 10: amino acid sequence of Homo sapiens EPH receptor A7 (EPHA7), mRNA. ACCESSION NM_{—}004440

SEQ ID NO: 11: nucleic acid sequence of Homo sapiens EPH receptor A8 (EPHA8), transcript variant 1, mRNA. ACCESSION NM_{—}020526

SEQ ID NO: 12: amino acid sequence of Homo sapiens EPH receptor A8 (EPHA8), transcript variant 1, mRNA. ACCESSION NM_{—}020526

SEQ ID NO: 13: nucleic acid sequence of Homo sapiens EPH receptor B1 (EPHB1), mRNA. ACCESSION NM_{—}004441

SEQ ID NO: 14: amino acid sequence of Homo sapiens EPH receptor B1 (EPHB1), mRNA. ACCESSION NM_{—}004441

SEQ ID NO: 15: nucleic acid sequence of Homo sapiens EPH receptor B2 (EPHB2), transcript variant 2, mRNA. ACCESSION NM_{—}004442

SEQ ID NO: 16: amino acid sequence of Homo sapiens EPH receptor B2 (EPHB2), transcript variant 2, mRNA. ACCESSION NM_{—}004442

SEQ ID NO: 17: nucleic acid sequence of Homo sapiens EPH receptor B3 (EPHB3), mRNA. ACCESSION NM_{—}004443

SEQ ID NO: 18: amino acid sequence of Homo sapiens EPH receptor B3 (EPHB3), mRNA. ACCESSION NM_{—}004443

SEQ ID NO: 19: nucleic acid sequence of Homo sapiens EPH receptor B4 (EPHB4), mRNA. ACCESSION NM_{—}004444

SEQ ID NO: 20: amino acid sequence of Homo sapiens EPH receptor B4 (EPHB4), mRNA. ACCESSION NM_{—}004444

SEQ ID NO: 21: nucleic acid sequence of Homo sapiens EPH receptor B6 (EPHB6), mRNA. ACCESSION NM_{—}004445.

SEQ ID NO: 22: amino acid sequence of Homo sapiens EPH receptor B6 (EPHB6), mRNA. ACCESSION NM_{—}004445.

SEQ ID NO: 23: nucleic acid sequence of Homo sapiens v-kit Hardy-Zuckerman 4 feline sarcoma viral oncogene homolog (KIT), mRNA. ACCESSION NM_{—}000222.

SEQ ID NO: 24: amino acid sequence of Homo sapiens v-kit Hardy-Zuckerman 4 feline sarcoma viral oncogene homolog (KIT), mRNA. ACCESSION NM_{—}000222.

SEQ ID NO: 25: nucleic acid sequence of Homo sapiens anaplastic lymphoma kinase (Ki-1) (ALK), mRNA. ACCESSION NM_{—}004304.

SEQ ID NO: 26: amino acid sequence of Homo sapiens anaplastic lymphoma kinase (Ki-1) (ALK), mRNA. ACCESSION NM_{—}004304.

### BEST MODE FOR CARRYING OUT THE INVENTION

It should be understood throughout the present specification that articles for a singular form includes the concept of their plurality unless otherwise mentioned. Therefore, articles or adjectives for singular forms (e.g., “a”, “an”, “the”, etc. in English, and articles, adjectives, etc. in other languages) include the concept of their plurality unless otherwise specified. As such, the terms “a” or “an”, “one or more” and “at least one” can be used interchangeably herein. It is also to be noted that the terms “comprising,” “including,” and “having” can be used interchangeably. Furthermore, a compound “selected from the group consisting of, refers to one or more of the compounds in the list that follows, including mixtures (i.e. combinations) of two or more of the compounds. It should be also understood that terms used herein have definitions which are ordinarily used in the art unless otherwise mentioned. Therefore, all technical and scientific terms used herein have the same meanings as commonly understood by those skilled in the art. Otherwise, the present application (including definitions) takes precedence.

Before the present compounds, compositions, system, device and/or methods are disclosed and described, it is to be understood that this invention are not limited to specific synthetic methods, specific reagents or to laboratory or manufacturing techniques, as such may, of course, vary unless it is otherwise indicated. It is also to be understood that the terminology used herein are for the purposes of describing particular embodiments only and are not intended to be limiting.

### DEFINITION OF TERMS

Hereinafter, terms specifically used herein will be defined.

### (Biological Functions)

As used herein, the term “network of biological functions” refers to any network of parameters of a biological entity, such as genes, transcriptional factors, structural genes, cellular markers, cell surface markers, cell shapes, organelle shapes, cell mobility, enzyme activities, metabolite concentrations, and localization of cellular components and the like. Such networks may be, but are not limited to, a pathway of parameters such as genes, signal transduction pathway and the like.

As used herein, a “pathway” refers to any pathway of parameters of a biological entity. Such pathways may be, but are not limited to, a pathway of a drug stimulations and the like.

As used herein, the term “biological function” refers to any parameter which is related to and/or reflects the living state of a biological entity such as a cell. Such biological functions include, but are not limited to, transcriptional factors, regulatory genes, structural genes, cellular markers, cell surface markers, cell shapes, organelle shapes, cell mobility, enzyme activities, metabolite concentrations and the localization of cellular components. Such biological functions may be measured by using a functional reporter which is specific to its function. As used herein the term “specific” in terms of the biological function refers to the relationship between a biological function and a functional reporter, wherein a change in the functional reporter is related to the change in the state of the biological function.

As used herein, the term “perturbation agent” or “stimulant (or stimulation agent)” refers to any agent that causes perturbation in a biological entity. Such perturbation agents or stimulants (or stimulation agents) include, but are not limited to, for example, RNA (e.g. siRNA, shRNA, miRNA, ribozyme), chemical compounds, cDNA, antibodies, polypeptides, light, sound, pressure change, radiation, heat, gas, and the like, particularly siRNA capable of specifically regulating a function of the said functional reporter is preferred, since such siRNA specifically targets the function in a biological entity such as a cell.

As used herein, the term “functional reporter” refers to an agent which changes the signal of a biological function to a measurable signal, such as light, expression of protein, production of metabolite, change in color, fluorescence, chemilunescence, and the like.

### <Mathematical Analysis>

As used herein, the term “set theory” refers to a theory as used and understood in the art, and the branch of pure mathematics that deals with the nature and the relationships of sets. A mathematical formalization of the theory of “sets” (aggregates or collections) of objects (“elements” or “members”). Many mathematicians use the set theory as the basis for all other mathematics. Such set theory includes the analysis of members into sets and classification of sets into inclusion, independent and intersection, and the like.

As used herein, the term “set” is used as in the set theory in the art, and refers to a group of members or elements.

As used herein, the term “member”, “cardinality” or “element” is interchangeably used to refer to a basic unit of a set. In the present invention, a functional reporter can be regarded as a set, and a perturbation agent or information/data/result derived there from, can be regarded as a member.

As used herein, the term “inclusion” refers to a relationship between two sets where all the members of one set is included in the other set.

As used herein, the term “independent” refers to a relationship between two groups, where all members of one set are not included in the other set and vice versa.

As used herein, the term “intersection” refers to a relationship between two sets where some members of one set are included and some are not, and vice versa, therefore there is an overlap set between the two sets.

As used herein, the term “network relationship” refers to a relationship between members of a network. Such a relationship may be presented in a map of members with arrows, which shows the direction of influence of one member on the other.

As used herein, the term “parallel” are used for the relationship between two parameters, referring to the state where the two parameters are located in different pathways in a network.

As used herein, the term “downstream” are used for the relationship between two parameters, referring to the state where one of the two parameters is located downstream of the other in a pathway or a network.

As used herein, the term “upstream” is used for the relationship between two parameters, referring to the state where one of the two parameters is located upstream of the other in a pathway or a network.

As used herein, the term “common” refers to a state where two parameters are in the same relationship for a function or any other parameter of a biological entity.

As used herein, the phrase “equally targeting” refers to a condition of distributing perturbation agents or stimulants, where the perturbation agents or stimulants to be introduced have substantially the same effects on the targets of interest. In the present invention, two or more perturbation agents are usually used to change the network structure of a biological entity such as a cell, it is preferable to use such equally targeting perturbation agents or stimulants.

As used herein, the term “threshold” refers to a specific value for evaluating whether a function is activated or suppressed. Such a threshold may be determined experimentally, empirically or theoretically. Thresholds may be arbitrarily selected for certain cases.

### (Biology)

As used herein, the term “biological entity” refers to any entity which is biologically living. Examples of such biological entities include living organisms, organ, tissue, cell, microorganisms such as bacteria, virus and the like.

The term “cell” is herein used in its broadest sense in the art, referring to the structural unit of a tissue of a multicellular organism, which is capable of self replicating, has genetic information and a mechanism for expressing it and is surrounded by a membrane structure which isolates the cell from the outside. Cells used herein may be either naturally-occurring cells or artificially modified cells (e.g., fusion cells, genetically modified cells, etc.). Examples of cell sources include, but are not limited to, a single-cell culture; an embryo, blood, or body tissue of normally-grown transgenic animal; a mixture of cells derived from normally-grown cell lines; and the like.

Cells used herein may be derived from any organism (e.g., any unicellular organisms (e.g., bacteria and yeast) or any multicellular organisms (e.g., animals (e.g., vertebrates and invertebrates) and plants (e.g., monocotyledons and dicotyledons, etc.)). For example, cells used herein are derived from a vertebrate (e.g., Myxiniformes, Petronyzoniformes, Chondrichthyes, Osteichthyes, amphibian, reptilian, avian, mammalian, etc.), more preferably mammalian (e.g., monotremata, marsupialia, edentate, dermoptera, chiroptera, carnivore, insectivore, proboscidea, perissodactyla, artiodactyla, tubulidentata, pholidota, sirenia, cetacean, primates, rodentia, lagomorpha, etc.). In one embodiment, cells are derived from Primates (e.g., chimpanzee, Japanese monkey, human) are used. The above-described cells may be either stem cells or somatic cells. Also, the cells may be adherent cells, suspended cells, tissue forming cells and mixtures thereof. The cells may be used for transplantation.

Any organs may be targeted by the present invention. A biological entity such as a tissue or cell targeted by the present invention may be derived from any organs. As used herein, the term “organ” refers to a morphologically independent structure localized at a particular portion of an individual organism in which a certain function is performed. In a multicellular organisms (e.g., animals, plants), an organ consists of a several tissues spatially arranged in a particular manner, each tissue being composed of a number of cells. An example of such an organ includes an organ relating to the vascular system. In one embodiment, organs targeted by the present invention include, but are not limited to, skin, blood vessel, cornea, kidney, heart, liver, umbilical cord, intestine, nerve, lung, placenta, pancreas, brain, peripheral limbs, retina and the like. Examples of cells differentiated from pluripotent cells includes epidermic cells, pancreatic parenchymal cells, pancreatic duct cells, hepatic cells, blood cells, cardiac muscle cells, skeletal muscle cells, osteoblasts, skeletal myoblasts, neurons, vascular endothelial cells, pigment cells, smooth muscle cells, fat cells, bone cells, cartilage cells and the like.

As used herein, the term “tissue” refers to an aggregate of cells having substantially the same function and/or forms in a multicellular organism. “Tissue” is typically an aggregate of cells in the same origin, but may be an aggregate of cells of a different origins as long as the cells have the same function and/or forms. Therefore, tissues used herein may be composed of an aggregate of cells of two or more different origins. Typically, a tissue constitutes a part of an organ. Animal tissues are separated into epithelial tissue, connective tissue, muscular tissue, nervous tissue and the like, on a morphological, functional, or developmental basis. Plant tissues are roughly separated into meristematic tissue and permanent tissue according to the developmental stage of the cells constituting the tissue. Alternatively, tissues may be separated into single tissues and composite tissues according to the type of cells constituting the tissue. Thus, tissues are separated into various categories.

As used herein, the term “isolated” means that naturally accompanying material is at least reduced, or preferably substantially completely eliminated, in normal circumstances. As used herein, an isolated biological entity can be targeted by the present invention. Therefore, the term “isolated cell” refers to a cell substantially free from other accompanying substances (e.g., other cells, proteins, nucleic acids, etc.) in natural circumstances. The term “isolated” in relation to nucleic acids or polypeptides means that, for example, the nucleic acids or the polypeptides are substantially free from cellular substances or culture media when they are produced by recombinant DNA techniques; or precursory chemical substances or other chemical substances when they are chemically synthesized. Isolated nucleic acids are preferably free from sequences which are naturally flanking the nucleic acid within an organism from which the nucleic acid are derived (i.e., sequences positioned at the 5′ terminus and the 3′ terminus of the nucleic acid). Preferably, an isolated cell is used for analysis of the present invention.

As used herein, the term “established” in relation to cells refers to a state of a cell in which a particular property (such as pluripotency) of the cell are maintained and the cell undergoes stable proliferation under culture conditions. In the present invention, such an established cell may be used.

As used herein, the term “state” refers to a condition concerning various parameters of a biological entity such as a cell (e.g., cell cycle, response to an external factor, signal transduction, gene expression, gene transcription, etc.). Examples of such a state include, but are not limited to, differentiated states, undifferentiated states, responses to external factors, cell cycles, growth states and the like. As used herein, the term “gene state” refers to any state associated with a gene (e.g., an expression state, a transcription state, etc.).

As used herein, the terms “differentiation” or “cell differentiation” refers to a phenomenon where two or more types of cells, having qualitative differences in forms and/or functions occurring in daughter cell populations, are derived from the division of a single cell. Therefore, “differentiation” includes a process during which a population (family tree) of cells, which do not originally have a specific detectable feature, acquire a feature, such as production of a specific protein, or the like. At present, cell differentiation is generally considered to be a state of a cell in which a specific group of genes in the genome are expressed. Cell differentiation can be identified by searching for intracellular or extracellular agents or conditions which elicit the above-described state of gene expression. Differentiated cells are stable in principle. Particularly, animal cells as once differentiated, they are rarely differentiated into other types of cells.

As used herein, the term “pluripotency” refers to a nature of a cell, i.e., an ability to differentiate into one or more, preferably two or more, tissues or organs. Therefore, the terms “pluripotent” and “undifferentiated” are herein used interchangeably unless otherwise mentioned. Typically, the pluripotency of a cell is limited during development, and in an adult, cells constituting a tissue or organ rarely alter to different cells, that is, the pluripotency is usually lost. Particularly, epithelial cells resist altering to other types of epithelial cells. Such alteration typically occurs in pathological conditions, and is called metaplasia. However, mesenchymal cells tend to easily undergo metaplasia, i.e., alter to other mesenchymal cells, with relatively simple stimuli. Therefore, mesenchymal cells have a high level of pluripotency. Embryonic stem cells have pluripotency, and tissue stem cells have pluripotency. Thus, the term “pluripotency” may include the concept of totipotency. An example of an in vitro assay for determining whether or not a cell has pluripotency, includes but is not limited to, culturing under conditions for inducing the formation and the differentiation of embryoid bodies. Examples of an in vivo assay for determining the presence or absence of pluripotency, include but are not limited to, implantation of a cell into an immunodeficient mouse so as to form teratoma, injection of a cell into a blastocyst so as to form a chimeric embryo, implantation of a cell into a tissue of an organism (e.g., injection of a cell into ascites) so as to undergo proliferation and the like. As used herein, one type of pluripotency is “totipotency”, which refers to an ability to be differentiated into all kinds of cells which constitute an organism. The idea of pluripotency encompasses totipotency. An example of a totipotent cell is a fertilized ovum. An ability to differentiate into one type of cell is called “unipotency”.

As used herein, the term “gene” refers to an element defining a genetic trait, which is a biological function of a biological entity. A gene is typically arranged in a given sequence on a chromosome or other extrachromosomal factor. A gene which defines the primary structure of a protein is called a structural gene. A gene which regulates the expression of a structural gene is called a regulatory gene (e.g., promoter). Genes herein includes structural genes and regulatory genes unless otherwise specified. Therefore, for example, the term “cyclin gene” typically includes the structural gene of cyclin and the promoter of cyclin. As used herein, “gene” may refer to “polynucleotide”, “oligonucleotide”, “nucleic acid”, and “nucleic acid molecule” and/or “protein”, “polypeptide”, “oligopeptide” and “peptide”. As used herein, “gene product” includes “polynucleotide”, “oligonucleotide”, “nucleic acid” and “nucleic acid molecule” and/or “protein”, “polypeptide”, “oligopeptide” and “peptide”, which are expressed by a gene. Those skilled in the art understands what a gene product is, according to the context.

As used herein, the term “homology”, in relation to a sequence (e.g., a nucleic acid sequence, an amino acid sequence, etc.) refers to the proportion of the identity between two or more gene sequences. Therefore, the greater the homology between two given genes, the greater the identity or similarity between their sequences. Whether or not two genes have homology is determined by comparing their sequences directly or by a hybridization method under stringent conditions. When two gene sequences are directly compared with each other, these genes have a homology if the DNA sequences of the genes have represented at least 50% identity, preferably at least 70% identity, more preferably at least 80%, 90%, 95%, 96%, 97%, 98%, or 99% identity with each other. As used herein, the term “similarity” in relation to a sequence (e.g., a nucleic acid sequence, an amino acid sequence or the like) refers to the proportion of identity between two or more sequences when conservative substitution is regarded as positive (identical) in the above-described homology. Therefore, homology and similarity differ from each other in the presence of conservative substitutions. If no conservative substitutions are present, homology and similarity have the same value. Such homologous genes and the like may be used as the same function in a network, if applicable, and may be used as different perturbation agents and the like, if applicable.

The terms “protein”, “polypeptide”, “oligopeptide” and “peptide” as used herein, have the same meaning and refer to an amino acid polymer of any length. This polymer may be a straight, branched or cyclic chain polymer. An amino acid may be a naturally-occurring, nonnaturally-occurring amino acid or a variant amino acid. The term may include those assembled into a composite of a plurality of polypeptide chains. The term also includes a naturally-occurring or artificially modified amino acid polymer. Such modification includes, for example, disulfide bond formation, glycosylation, lipidation, acetylation, phosphorylation or any other manipulation or modification (e.g., conjugation with a labeling moiety). This definition encompasses a polypeptide containing at least one amino acid analog (e.g., nonnaturally-occurring amino acid, etc.), a peptide-like compound (e.g., peptoid), and other variants known in the art, for example. Gene products, such as extracellular matrix proteins (e.g., fibronectin, etc.) are usually in the form of a polypeptide. Polypeptides used in the present invention may be produced by, for example, cultivating primary culture cells producing the peptides or cell lines thereof, followed by separation or purification of the peptides from the culture supernatant. Alternatively, genetic manipulation techniques are used to incorporate a gene which encodes a polypeptide of interest into an appropriate expression vector, transform an expression host with the vector and collect recombinant polypeptides from the culture supernatant of the transformed cells. The above-described host cell may be any host cells conventionally used in genetic manipulation techniques, as long as they can express a polypeptide of interest while maintaining the physiological activity of the peptide (e.g., E. coli, yeast, an animal cell, etc.). Polypeptides derived from the thus-obtained cells may have at least one amino acid substitution, addition, and/or deletion or at least one sugar chain substitution, addition, and/or deletion as long as they have substantially the same function as that of naturally-occurring polypeptides.

The terms “polynucleotide”, “oligonucleotide”, “nucleic acid molecule” and “nucleic acid”, as used herein, have the same meaning and refer to a nucleotide polymer having any length. These terms also includes an “oligonucleotide derivative” or a “polynucleotide derivative”. An “oligonucleotide derivative” or a “polynucleotide derivative” includes a nucleotide derivative, or refers to an oligonucleotide or a polynucleotide having different linkages between nucleotides from typical linkages, which are interchangeably used. Examples of such an oligonucleotide specifically include 2′-O-methyl-ribonucleotide, an oligonucleotide derivative in which a phosphodiester bond in an oligonucleotide is converted to a phosphorothioate bond, an oligonucleotide derivative in which a phosphodiester bond in an oligonucleotide is converted to a N3′-P5′ phosphoroamidate bond, an oligonucleotide derivative in which a ribose and a phosphodiester bond in an oligonucleotide are converted to a peptide-nucleic acid bond, an oligonucleotide derivative in which uracil in an oligonucleotide is substituted with C-5 propynyl uracil, an oligonucleotide derivative in which uracil in an oligonucleotide is substituted with C-5 thiazole uracil, an oligonucleotide derivative in which cytosine in an oligonucleotide is substituted with C-5 propynyl cytosine, an oligonucleotide derivative in which cytosine in an oligonucleotide is substituted with phenoxazine-modified cytosine, an oligonucleotide derivative in which ribose in DNA is substituted with 2′-O-propyl ribose, and an oligonucleotide derivative in which ribose in an oligonucleotide is substituted with 2′-methoxyethoxy ribose. Unless otherwise indicated, a particular nucleic acid sequence also implicitly encompasses conservatively-modified variants thereof (e.g. degenerate codon substitutions) and complementary sequences as well as the sequence explicitly indicated. Specifically, degenerate codon substitutions may be produced by generating sequences in which the third position of one or more selected (or all) codons are substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). A gene encoding an extracellular matrix protein (e.g., fibronectin, etc.) or the like is usually in the form of a polynucleotide. A molecule to be transfected is in the form of a polynucleotide.

As used herein, the term “corresponding”, is used for the relationship between a functional reporter and function, referring to a state where the signal derived from a functional reporter of interest reflects the state of a function. Therefore, one can determine the state of such function based on the signal of the functional reporter corresponding to the function. For example, a gene expressing a fluorescent protein linked under a transcriptional factor is said to be a functional reporter corresponding to the transcriptional factor and the like.

As used herein, the term “corresponding” amino acid or nucleic acid refers to an amino acid or nucleotide in a given polypeptide or polynucleotide molecule which has, or is anticipated to have, a function similar to that of a predetermined amino acid or nucleotide in a polypeptide or polynucleotide as a reference for comparison. Particularly, in the case of enzyme molecules, the term refers to an amino acid which is present at a similar position in an active site and similarly contributes to a catalytic activity. For example, in the case of antisense molecules for a certain polynucleotide, the term refers to a similar portion in an ortholog corresponding to a particular portion of the antisense molecule.

As used herein, the term “corresponding” gene (e.g., a polypeptide or polynucleotide molecule) refers to a gene in a given species which has, or is anticipated to have, a function similar to that of a predetermined gene in a species as a reference for comparison. When there are a plurality of genes having such a function, the term refers to a gene having the same evolutionary origin. Therefore, a gene corresponding to a given gene may be an ortholog of the given gene. Therefore, genes corresponding to mouse cyclin genes can be found in other animals. Such a corresponding gene can be identified by techniques well known in the art. Therefore, for example, a corresponding gene in a given animal can be found by searching a sequence database of the animal (e.g., human, rat) using the sequence of a reference gene (e.g., mouse cyclin gene, etc.) as a query sequence.

As used herein, the term “fragment”, with respect to a polypeptide or polynucleotide, refer to a polypeptide or polynucleotide having a sequence length ranging from 1 to n−1 with respect to the full length of the reference polypeptide or polynucleotide (of length n). This length of the fragment can be appropriately changed depending on the purpose. For example, in the case of polypeptides, the lower limits of the length of the fragment includes 3, 4, 5, 6, 7, 8, 9, 10, 15, 20, 25, 30, 40, 50 or more nucleotides. Lengths represented by integers, which are not herein specified (e.g., 11 and the like), may be appropriate as a lower limit. For example, in the case of polynucleotides, the lower limits of the length of the fragment includes 5, 6, 7, 8, 9, 10, 15, 20, 25, 30, 40, 50, 75, 100 or more nucleotides. Lengths represented by integers, which are not herein specified (e.g., 11 and the like), may be appropriate as a lower limit. As used herein, the length of polypeptides or polynucleotides can be represented by the number of amino acids or nucleic acids, respectively. However, the above-described numbers are not absolute. The above-described numbers as the upper or lower limits are intended to include some greater or smaller numbers (e.g., ±10%), as long as the same function is maintained. For this purpose, “about” may be herein put ahead of the numbers. However, it should be understood that the interpretation of numbers is not affected by the presence or absence of “about” in the present specification.

As used herein, the term “biological activity” refers to activity possessed by an agent (e.g., a polynucleotide, a protein, etc.) within an organism, including activities exhibiting various functions (e.g., transcription promoting activity, etc.). For example, when a certain factor is an enzyme, the biological activity thereof includes its enzyme activity. In another example, when a certain factor is a ligand, the biological activity thereof includes the binding of the ligand to a receptor corresponding thereto. The above-described biological activity can be measured by techniques well-known in the art.

As used herein, the term “search” indicates that a given nucleic acid sequence is utilized to find other nucleic acid base sequences having a specific function and/or property either electronically, biologically or by using other methods. Examples of an electronic search includes, but is not limited to, BLAST (Altschul et al., J. Mol. Biol. 215:403-410 (1990)), FASTA (Pearson & Lipman, Proc. Natl. Acad. Sci., USA 85:2444-2448 (1988)), Smith and Waterman method (Smith and Waterman, J. Mol. Biol. 147:195-197 (1981)), and Needleman and Wunsch method (Needleman and Wunsch, J. Mol. Biol. 48:443-453 (1970)) and the like. Examples of a biological search includes, but is not limited to, a macroarray in which genomic DNA is attached to a nylon membrane or the like, or a microarray (microassay) in which genomic DNA is attached to a glass plate under stringent hybridization conditions, PCR and in situ hybridization and the like. Such a search may be conducted by using a method or system of the present invention.

As used herein, the term “probe” refers to a substance for use in searching, which is used in a biological experiment, such as in vitro and/or in vivo screening or the like, including but not being limited to, for example, a nucleic acid molecule having a specific base sequence or a peptide containing a specific amino acid sequence.

Examples of a nucleic acid molecule as a common probe include one having a nucleic acid sequence, having a length of at least 8 contiguous nucleotides, which is homologous or complementary to the nucleic acid sequence of a gene of interest. Such a nucleic acid sequence may be preferably a nucleic acid sequence having a length of at least 9 contiguous nucleotides, more preferably a length of at least 10 contiguous nucleotides, and even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 20 contiguous nucleotides, a length of at least 25 contiguous nucleotides, a length of at least 30 contiguous nucleotides, a length of at least 40 contiguous nucleotides, or a length of at least 50 contiguous nucleotides. A nucleic acid sequence used as a probe includes a nucleic acid sequence having at least 70% homology to the above-described sequence, more preferably at least 80%, and even more preferably at least 90% or at least 95%.

As used herein, the term “primer” refers to a substance required to initiate a reaction of a macromolecule compound that is synthesized in an enzymatic reaction. In a reaction for synthesizing nucleic acid molecules, nucleic acid molecules (e.g., DNA, RNA, or the like) which are complementary to part of a macromolecule compound to be synthesized may be used.

A nucleic acid molecule which is ordinarily used as a primer includes one that has a nucleic acid sequence having a length of at least 8 contiguous nucleotides, which is complementary to the nucleic acid sequence of a gene of interest. Such a nucleic acid sequence preferably has a length of at least 9 contiguous nucleotides, more preferably a length of at least 10 contiguous nucleotides, even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 16 contiguous nucleotides, a length of at least 17 contiguous nucleotides, a length of at least 18 contiguous nucleotides, a length of at least 19 contiguous nucleotides, a length of at least 20 contiguous nucleotides, a length of at least 25 contiguous nucleotides, a length of at least 30 contiguous nucleotides, a length of at least 40 contiguous nucleotides, and a length of at least 50 contiguous nucleotides. A nucleic acid sequence used as a primer includes a nucleic acid sequence having at least 70% homology to the above-described sequence, more preferably at least 80%, even more preferably at least 90%, and most preferably at least 95%. An appropriate sequence as a primer may vary depending on the property of the sequence to be synthesized (amplified). Those skilled in the art, can design an appropriate primer depending on the sequence of interest. Such primer design is well known in the art and may be performed manually or using a computer program (e.g., LASERGENE, Primer Select, DNAStar).

As used herein, the term “epitope” refers to an antigenic determinant. Therefore, the term “epitope” includes a set of amino acid residues which are involved in the recognition of a particular immunoglobulin, or in the context of T cells, those residues necessary for the recognition by the T cell receptor proteins and/or Major Histocompatibility Complex (MHC) receptors. This term is also used interchangeably with “antigenic determinant” or “antigenic determinant site”. In the field of immunology, in vivo or in vitro, an epitope is a feature of a molecule (e.g., primary, secondary and tertiary peptide structure, and charge) that forms a site recognized by an immunoglobulin, T cell receptor or HLA molecule. An epitope including a peptide comprises 3 or more amino acids in a spatial conformation which is unique to the epitope. Generally, an epitope consists of at least 5 such amino acids, and more ordinarily, consists of at least 6, 7, 8, 9 or 10 such amino acids. The greater the length of an epitope, the more similar the epitope to the original peptide, i.e., longer epitopes are generally preferable. This is not necessarily the case when the conformation is taken into account. Methods of determining the spatial conformation of amino acids are known in the art and include, for example, X-ray crystallography and 2-dimensional nuclear magnetic resonance spectroscopy. Furthermore, the identification of epitopes in a given protein is readily accomplished using techniques well known in the art. See, also, Geysen et al., Proc. Natl. Acad. Sci. USA (1984) 81: 3998 (general method of rapidly synthesizing peptides to determine the location of immunogenic epitopes in a given antigen); U.S. Pat. No. 4,708,871 (procedures for identifying and chemically synthesizing epitopes of antigens); and Geysen et al., Molecular immunology (1986) 23: 709 (technique for identifying peptides with high affinity for a given antibody). Antibodies which recognize the same epitope can be identified in a simple immunoassay. Thus, methods for determining epitopes, including a peptide are well known in the art. Such an epitope can be determined by using a well-known, common technique by those skilled in the art if the primary nucleic acid or amino acid sequence of the epitope is provided.

Therefore, an epitope including a peptide requires a sequence having a length of at least 3 amino acids, preferably at least 4 amino acids, more preferably at least 5 amino acids, at least 6 amino acids, at least 7 amino acids, at least 8 amino acids, at least 9 amino acids, at least 10 amino acids, at least 15 amino acids, at least 20 amino acids, and 25 amino acids. Epitopes may be linear or conformational.

As used herein, the term “biological molecule” refers to molecules, or aggregates of molecules, relating to an organism and aggregates of organisms. As used herein, the term “biological” or “organism” refers to a biological organism, including but being not limited to, an animal, a plant, a fungus, a virus and the like. Biological molecules include molecules extracted from an organism and aggregations thereof, though the present invention is not limited to this. Any molecules or aggregates of molecules relating to an organism and aggregates of organisms fall within the definition of a biological molecule. Therefore, low molecular weight molecules (e.g., low molecular weight molecule ligands, etc.) capable of being used as medicaments fall within the definition of a biological molecule as long as an effect on an organism is intended. Examples of such a biological molecule include, but are not limited to, proteins, polypeptides, oligopeptides, peptides, polynucleotides, oligonucleotides, nucleotides, nucleic acids (e.g., DNA such as cDNA and genomic DNA; RNA such as mRNA), polysaccharides, oligosaccharides, lipids, low molecular weight molecules (e.g., hormones, ligands, information transmitting substances, low molecular weight organic molecules, etc.), and composite molecules thereof and aggregations thereof (e.g., glycolipids, glycoproteins, lipoproteins, etc.) and the like. A biological molecule may include a cell itself or a portion of tissue as long as it is intended to be introduced into a cell. Typically, a biological molecule may be a nucleic acid, a protein, a lipid, a sugar, a proteolipid, a lipoprotein, a glycoprotein, a proteoglycan or the like. Preferably, a biological molecule may include a nucleic acid (DNA or RNA) or a protein. In an embodiment, a biological molecule is a nucleic acid (e.g., genomic DNA or cDNA, or DNA synthesized by PCR or the like). In another embodiment, a biological molecule may be a protein. Such a biological molecule may be a hormone or a cytokine.

As used herein, the term “receptor” refers to a molecule which is present on cells within nuclei, or the like, is capable of binding to an extracellular or intracellular agent where the binding mediates signal transduction. Receptors are typically in the form of proteins. The binding partner of a receptor is usually referred to as a ligand.

As used herein, the term “agonist” refers to an agent which binds to the receptor of a certain biologically acting substance (e.g., ligand, etc.), and has the same or similar function as the function of the substance.

As used herein, the term “antagonist” refers to a factor which competitively binds to the receptor of a certain biologically acting substance (ligand), and does not produce physiological action via the receptor. Antagonists include antagonist drugs, blockers, inhibitors and the like.

As used herein, the term “agent” may be any substance or other entity (e.g., energy, such as light, radiation, heat, electricity, or the like) as long as the intended purpose can be achieved. Examples of such a substance include but are not limited to, proteins, polypeptides, oligopeptides, peptides, polynucleotides, oligonucleotides, nucleotides, nucleic acids (e.g., DNA such as cDNA, genomic DNA, or the like, and RNA such as mRNA), polysaccharides, oligosaccharides, lipids, low molecular weight organic molecules (e.g., hormones, ligands, information transfer substances, molecules synthesized by combinatorial chemistry, low molecular weight molecules (e.g., pharmaceutically acceptable low molecular weight ligands and the like) and the like) and the combinations of these molecules. Examples of an agent specific to a polynucleotide include, but are not limited to, representatively, a polynucleotide having a sequence complementarily to the sequence of the polynucleotide with a predetermined sequence homology (e.g., 70% or more sequence identity), a polypeptide such as a transcriptional agent binding to a promoter region and the like. Examples of an agent specific to a polypeptide include, but are not limited to, representatively, an antibody specifically directed to the polypeptide or derivatives or analogs thereof (e.g., single chain antibody), a specific ligand or receptor when the polypeptide is a receptor or ligand, a substrate when the polypeptide is an enzyme and the like.

As used herein, the term “agent binding specifically to” a certain agent such as a nucleic acid molecule or polypeptide refers to an agent which has a level of binding to the nucleic acid molecule or polypeptide equal to or higher than a level of binding to other nucleic acid molecules or polypeptides. Examples of such an agent include, but are not limited to, when a target is a nucleic acid molecule, a nucleic acid molecule having a complementary sequence of a nucleic acid molecule of interest, a polypeptide capable of binding to a nucleic acid sequence of interest (e.g., a transcription agent, etc.) and the like, and when a target is a polypeptide, an antibody, a single chain antibody, either of a pair of a receptor and a ligand, either of a pair of an enzyme and a substrate, and the like.

As used herein, the term “compound” refers to any identifiable chemical substance or molecule, including but not limited to, a low molecular weight molecule, a peptide, a protein, a sugar, a nucleotide or a nucleic acid. Such a compound may be a naturally-occurring product or a synthetic product.

As used herein, the term “low molecular weight organic molecule” refers to an organic molecule having a relatively small molecular weight. Usually, the low molecular weight of an organic molecule refers to a molecular weight of about 1,000 or less, or alternatively may refer to a molecular weight of more than 1,000. Low molecular weight organic molecules can be ordinarily synthesized by methods known in the art or combinations thereof. These low molecular weight organic molecules may be produced by organisms. Examples of the low molecular weight organic molecules include, but are not limited to, hormones, ligands, information transfer substances, synthesized by combinatorial chemistry, pharmaceutically acceptable low molecular weight molecules (e.g., low molecular weight ligands and the like) and the like.

As used herein, the term “contact” refers to the direct or indirect placement of a compound, physically close to the polypeptide or polynucleotide of the present invention. Polypeptides or polynucleotides may be present in a number of buffers, salts, solutions and the like. The term “contact” includes placement of a compound in a beaker, a microtiter plate, a cell culture flask, a microarray (e.g., a gene chip) or the like which contains a polypeptide encoded by a nucleic acid or a fragment thereof.

As used herein, the term “antibody” encompasses polyclonal antibodies, monoclonal antibodies, human antibodies, humanized antibodies, polyfunctional antibodies, chimeric antibodies, anti-idiotype antibodies, fragments thereof (e.g., F(ab′)2 and Fab fragments) and other recombinant conjugates. These antibodies may be fused with an enzyme (e.g., alkaline phosphatase, horseradish peroxidase, α-galactosidase and the like) via a covalent bond or by recombination. Antibodies can be used as a perturbation agent in the present invention.

As used herein, the term “antigen” refers to any substrate to which an antibody molecule may specifically bind. As used herein, the term “immunogen” refers to an antigen capable of initiating activation of an antigen-specific immune response of a lymphocyte. Antigens can be used as a perturbation agent in the present invention.

In a given protein molecule, a given amino acid may be substituted with another amino acid in a structurally important region (such as a cationic region or a substrate molecule binding site) without a clear reduction or loss of interactive binding ability. A given biological function of a protein is defined by the interactive ability or other property of the protein. Therefore, a particular amino acid substitution may be performed in an amino acid sequence, or at the DNA sequence level, to produce a protein which maintains the original property after the substitution. Thus, these various modifications of peptides, as disclosed herein, and DNA encoding such peptides may be performed without a clear loss of biological activity.

When the above-described modifications are designed, the hydrophobicity indices of amino acids may be taken into consideration. The hydrophobic amino acid indices play an important role in providing a protein with an interactive biological function, which are generally recognized in the art (Kyte, J. and Doolittle, R. F., J. Mol. Biol. 157 (1):105-132, 1982). The hydrophobic property of an amino acid contributes to the secondary structure of a protein and then regulates interactions between the protein and other molecules (e.g., enzymes, substrates, receptors, DNA, antibodies, antigens, etc.). Each amino acid is given a hydrophobicity index based on the hydrophobicity and charge properties thereof as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamic acid (−3.5); glutamine (−3.5); aspartic acid (−3.5); asparagine (−3.5); lysine (−3.9); and arginine (−4.5).

It is well known that if a given amino acid is substituted with another amino acid having a similar hydrophobicity index, the protein may still have a biological function similar to that of the original protein (e.g., a protein having an equivalent enzymatic activity). For such an amino acid substitution, the hydrophobicity index is preferably within ±2, more preferably within ±1, and even more preferably within ±0.5. It is understood in the art that such an amino acid substitution based on hydrophobicity is efficient. As described in U.S. Pat. No. 4,554,101, amino acid residues are given the following hydrophilicity indices: arginine (+3.0); lysine (+3.0); aspartic acid (+3.0±1); glutamic acid (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid may be substituted with another amino acid which has a similar hydrophilicity index and can still provide a biological equivalent. For such an amino acid substitution, the hydrophilicity index is preferably within ±2, more preferably ±1, and even more preferably ±0.5.

### (Devices and Solid Phase Supports)

As used herein, the term “device” refers to a part which constitutes the whole or a portion of an apparatus, and comprises a support (preferably, a solid phase support) and a target substance carried thereon. Examples of such a device include, but are not limited to, chips, arrays, microtiter plates, cell culture plates, Petri dishes, films, beads and the like. Such a device may constitute a system of the present invention. In particular, such a device may be used as means for obtaining information on at least two functional reporters in said biological entity, wherein the functional reporters reflect a biological function.

As used herein, the term “support” refers to a material which can fix a substance, such as a biological molecule. Such a support may be made from any fixing material which has a capability of binding to a biological molecule as used herein via covalent or noncovalent bonds, or which may be induced to have such a capability.

Examples of materials used for supports include any material capable of forming a solid surface, such as but without limitations, glass, silica, silicon, ceramics, silicon dioxide, plastics, metals (including alloys), naturally-occurring and synthetic polymers (e.g., polystyrene, cellulose, chitosan, dextran, and nylon) and the like. A support may be formed of layers made of a plurality of materials. For example, a support may be made of an inorganic insulating material, such as glass, quartz glass, alumina, sapphire, forsterite, silicon oxide, silicon carbide, silicon nitride or the like. A support may be made of an organic material such as polyethylene, ethylene, polypropylene, polyisobutylene, polyethylene terephthalate, unsaturated polyester, fluorine-containing resin, polyvinyl chloride, polyvinylidene chloride, polyvinyl acetate, polyvinyl alcohol, polyvinyl acetal, acrylic resin, polyacrylonitrile, polystyrene, acetal resin, polycarbonate, polyamide, phenol resin, urea resin, epoxy resin, melamine resin, styrene-acrylonitrile copolymer, acrylonitrile-butadiene-styrene copolymer, silicone resin, polyphenylene oxide, polysulfone and the like. Also in the present invention, nitrocellulose film, nylon film, PVDF film or the like, which are used in blotting, may be used as a material for a support. When a material constituting a support is in the solid phase, such as a support is herein particularly referred to as a “solid phase support”. A solid phase support may be herein in the form of a plate, a microwell plate, a chip, a glass slide, a film, beads, a metal (surface) or the like. A support may not be coated or may be coated.

As used herein, the term “liquid phase” has the same meaning as commonly understood by those skilled in the art, typically referring to a state in solution.

As used herein, the term “solid phase” has the same meaning as commonly understood by those skilled in the art, typically referring to a solid state. As used herein, liquid and solid may be collectively referred to as a “fluid”.

As used herein, the term “substrate” refers to a material (preferably, solid) which is used to construct a chip or an array, according to the present invention. Therefore, substrates are included in the concept of plates. Such a substrate may be made from any solid material which has a capability of binding to a biological molecule as used herein via covalent or noncovalent bonds. The substrate may also be induced to have such a capabilities.

Examples of materials used for plates and substrates include any material capable of forming a solid surface, such as and without limitation, glass, silica, silicon, ceramics, silicon dioxide, plastics, metals (including alloys), naturally-occurring and synthetic polymers (e.g., polystyrene, cellulose, chitosan, dextran, and nylon) and the like. A support may be formed of layers made of a plurality of materials. For example, a support may be made of an inorganic insulating material, such as glass, quartz glass, alumina, sapphire, forsterite, silicon oxide, silicon carbide, silicon nitride or the like. A support may be made of an organic material, such as polyethylene, ethylene, polypropylene, polyisobutylene, polyethylene terephthalate, unsaturated polyester, fluorine-containing resin, polyvinyl chloride, polyvinylidene chloride, polyvinyl acetate, polyvinyl alcohol, polyvinyl acetal, acrylic resin, polyacrylonitrile, polystyrene, acetal resin, polycarbonate, polyamide, phenol resin, urea resin, epoxy resin, melamine resin, styrene-acrylonitrile copolymer, acrylonitrile-butadiene-styrene copolymer, silicone resin, polyphenylene oxide, polysulfone and the like. A material preferable as a substrate varies depending on various parameters, such as a measuring device, and can be selected from the above-described various materials as appropriate by those skilled in the art. For transfection arrays, glass slides are preferable. Preferably, such a substrate may have a coating.

As used herein, the term “coating” in relation to a solid phase support or substrate, refers to an act of forming a film of a material on a surface of the solid phase support or substrate, and also refers to a film itself. Coating is performed for various purposes, such as, an improvement in the quality of a solid phase support and substrate (e.g., elongation of life span, improvement in resistance to hostile environment, such as resistance to acids, etc.), an improvement in affinity to a substance integrated with a solid phase support or substrate and the like. Various materials may be used for such coating, including, without limitation, biological substances (e.g., DNA, RNA, protein, lipid, etc.), polymers (e.g., poly-L-lysine, MAS (available from Matsunami Glass, Kishiwada, Japan), and hydrophobic fluorine resin), silane (APS (e.g., γ-aminopropyl silane, etc.)), metals (e.g., gold, etc.), in addition to the above-described solid phase support and substrate. The selection of such materials are within the technical scope of those skilled in the art and thus can be performed using techniques well known in the art. In one preferred embodiment, such a coating may be advantageously made of poly-L-lysine, silane (e.g., epoxy silane or mercaptosilane, APS (γ-aminopropyl silane), etc.), MAS, hydrophobic fluorine resin and a metal (e.g., gold, etc.). Such a material may be preferably a substance suitable for cells or objects containing cells (e.g., organisms, organs, etc.).

As used herein, the terms “chip” or “microchip” are used interchangeably to refer to a micro integrated circuit which has versatile functions and constitutes a portion of a system. Examples of a chip include, but are not limited to, DNA chips, protein chips and the like.

As used herein, the term “array” refers to a substrate (e.g., a chip, etc.) which has a pattern of a composition containing at least one (e.g., 1000 or more, etc.) target substance (e.g., DNA, proteins, transfection mixtures, etc.) which is arrayed. Among arrays, patterned substrates, having a small size, (e.g., 10×10 mm, etc.) are particularly referred to as microarrays. The terms “microarray” and “array” are used interchangeably. Therefore, a patterned substrate having a larger size than what is described above, may be referred to as a microarray. For example, an array comprises a set of desired transfection mixtures fixed to a solid phase surface or a film thereof. An array preferably comprises at least 10² antibodies of the same or different types, more preferably at least 10³, even more preferably at least 10⁴, and still even more preferably at least 10⁵. These antibodies are placed on a surface of up to 125×80 mm, more preferably 10×10 mm. An array includes, but is not limited to, a 96-well microtiter plate, a 384-well microtiter plate, a microtiter plate the size of a glass slide and the like. A composition to be fixed may contain one or a plurality of types of target substances. Such a number of target substance types may range from one to a number of spots, including and without limitation, about 10, about 100, about 500 and about 1,000.

As used herein, the term “transfection array” refers to an array which embodies transfection on each of the spots or addresses on the array. Such transfection may be conducted using the technology described herein and exemplified in the Examples.

As described above, any number of target substances (e.g., proteins, such as antibodies) may be provided on a solid phase surface or film, typically including no more than 10⁸ biological molecules per substrate, in another embodiment no more than 10⁷ biological molecules, no more than 10⁶ biological molecules, no more than 10⁵ biological molecules, no more than 10⁴ biological molecules, no more than 10³ biological molecules or no more than 10² biological molecules. A composition containing more than 10⁸ biological molecule target substances may be provided on a substrate. In these cases, the size of a substrate is preferably small. Particularly, the size of a spot of a composition containing target substances (e.g., proteins such as antibodies) may be as small as the size of a single biological molecule (e.g., 1 to 2 nm order). In some cases, the minimum area of a substrate may be determined based on the number of biological molecules on a substrate. A composition containing target substances, which are intended to be introduced into cells, are herein typically arrayed on and fixed via covalent bonds or physical interaction to a substrate in the form of spots having a size of 0.01 mm to 10 mm.

“Spots” of biological molecules may be provided on an array. As used herein, the term “spot” refers to a certain set of compositions containing target substances. As used herein, the term “spotting” refers to an act of preparing a spot of a composition containing a certain target substance on a substrate or plate. Spotting may be performed by any method, for example, pipetting or the like, or alternatively by using an automatic device. These methods are well known in the art.

As used herein, the term “address” refers to a unique position on a substrate, which may be distinguished from other unique positions. Addresses are appropriately associated with spots. Addresses can have any distinguishable shape, such that substances at each address may be distinguished from substances at other addresses (e.g., optically). A shape defining an address may be, for example and without limitation, a circle, an ellipse, a square, a rectangle, or an irregular shape. Therefore, the term “address” is used to indicate an abstract concept, while the term “spot” is used to indicate a specific concept. Unless it is necessary to distinguish them from each other, the terms “address” and “spot” may be herein used interchangeably.

The size of each address particularly depends on the size of the substrate, the number of addresses on the substrate, the amount of a composition containing target substances and/or available reagents, the size of microparticles and the level of resolution required for any method used for the array. The size of each address may be, for example, in the range of from 1-2 nm to several centimeters, though the address may have any size suited to an array.

The spatial arrangements and shapes which define an address are designed so that the microarray is suited to a particular application. Addresses may be densely arranged or sparsely distributed, or subgrouped into a desired pattern appropriate for a particular type of material to be analyzed.

Microarrays are widely reviewed in, for example, “Genomu Kino Kenkyu Purotokoru [Genomic Function Research Protocol] (Jikken Igaku Bessatsu [Special Issue of Experimental Medicine], Posuto Genomu Jidai no Jikken Koza 1 [Lecture 1 on Experimentation in Post-genome Era], “Genomu Ikagaku to korekarano Genomu Iryo [Genome Medical Science and Future Genome Therapy] (Jikken Igaku Zokan [Special Issue of Experimental Medicine]) and the like.

A vast amount of data can be obtained from a microarray. Therefore, data analysis software is important for the administration of the correspondence between clones and spots, data analysis and the like. Such software may be attached to various detection systems (e.g., Ermolaeva O. et al., (1998) Nat. Genet., 20: 19-23). The format of database includes, for example, GATC (genetic analysis technology consortium) proposed by Affymetrix.

Micromachining for arrays is described in, for example, Campbell, S. A. (1996), “The Science and Engineering of Microelectronic Fabrication”, Oxford University Press; Zaut, P. V. (1996), “Microarray Fabrication: a Practical Guide to Semiconductor Processing”, Semiconductor Services; Madou, M. J. (1997), “Fundamentals of Microfabrication”, CRC1 5 Press; Rai-Choudhury, P. (1997), “Handbook of Microlithography, Micromachining, & Microfabrication: Microlithography”; and the like, portions related thereto of which are herein incorporated by reference.

### (Detection)

In cell analysis or determination in the present invention, various detection methods and means can be used as long as they can be used to detect information attributed to a cell or a substance interacting therewith. Examples of such detection methods and means include, but are not limited to, visual inspection, optical microscopes, confocal microscopes, reading devices using a laser light source, surface plasmon resonance (SPR) imaging, electric signals, chemical or biochemical markers, these may be used singly or in combination. Examples of such a detecting device include, but are not limited to, fluorescence analyzing devices, spectrophotometers, scintillation counters, CCD, luminometers and the like. Any means capable of detecting a biological molecule may be used.

As used herein, the term “marker” or “biomarker” are interchangeable and used to refer to a biological agent for indicating a level or frequency of a substance or state of interest. Examples of such markers include, but are not limited to, nucleic acids encoding a gene, gene products, metabolic products, receptors, ligands, antibodies and the like.

Therefore, as used herein, the term “marker” in relation to a state of a cell refers to an agent (e.g., ligands, antibodies, complementary nucleic acids, etc.) interacting with intracellular factors which indicates the state of the cell (e.g., nucleic acids encoding a gene, gene products (e.g., mRNA, proteins, posttranscriptionally modified proteins, etc.), metabolic products, receptors, etc.) and, in addition, to the transcription control factors. In the present invention, such markers may be used to produce information which is in turn analyzed. Such markers may preferably interact with a factor of interest. As used herein, the term “specificity” in relation to a marker refers to a property of the marker which interacts with a molecule of interest to a significantly higher extent than it does with other similar molecules. Such markers are herein, preferably present within cells or may be present outside cells.

As used herein, the term “label” refers to a factor which distinguishes a molecule, or a substance of interest, from others (e.g., substances, energy, electromagnetic waves, etc.). Examples of labeling methods include, but are not limited to, RI (radioisotope) methods, fluorescence methods, biotinylation methods, chemoluminance methods and the like. When the above-described nucleic acid fragments and complementary oligonucleotides are labeled by fluorescence methods, the fluorescent substances, having different fluorescence emission maximum wavelengths, are used for labeling. The difference between each fluorescence emission at maximum wavelength may be preferably 10 nm or more. Any fluorescent substance which can bind to a base portion of a nucleic acid may be used, preferably including a cyanine dye (e.g., Cy3 and Cy5 in the Cy Dye™ series, etc.), a rhodamine 6G reagent, N-acetoxy-N2-acetyl amino fluorene (AAF), AAIF (iodine derivative of AAF) and the like. For example, fluorescent substances having a difference in fluorescence emission at maximum wavelength of 10 nm or more include a combination of Cy5 and a rhodamine 6G reagent, a combination of Cy3 and fluorescein, a combination of a rhodamine 6G reagent and fluorescein and the like. In the present invention, such labels can be used to alter a sample of interest so that the sample can be detected by detecting means. These alterations are known in the art. Thus, those skilled in the art can perform such alteration using a method appropriate for a label and a sample of interest.

As used herein, the term “interaction” refers to and without limitation, hydrophobic interactions, hydrophilic interactions, hydrogen bonds, Van der Waals forces, ionic interactions, nonionic interactions, electrostatic interactions and the like.

As used herein, the term “interaction level” in relation to the interaction between two substances (e.g., cells, etc.) refers to the extent or frequency of interaction between the two substances. Such an interaction level can be measured by methods well known in the art. For example, the number of cells which are fixed and actually perform an interaction is counted directly or indirectly (e.g., the intensity of reflected light) for example, without limitation, by using an optical microscope, a fluorescence microscope, a phase-contrast microscope, or the like, or alternatively by staining cells with a marker, an antibody, a fluorescent label or the like, specific thereto and measuring the intensity thereof. Such a level can be displayed directly from a marker or indirectly via a label. Based on the measured value of such a level, the number or frequency of genes, which are actually transcribed or expressed in a certain spot, can be calculated.

### (Presentation and Display)

As used herein, the terms “display” and “presentation” are used interchangeably to refer to an act of providing information obtained by a method of the present invention or information derived there from, directly or indirectly, or in an information-processed form. Examples of such displayed forms include, but are not limited to various methods, such as graphs, photographs, tables, animations, and the like. Such techniques are described in, for example, METHODS IN CELL BIOLOGY, VOL. 56, ed. 1998, pp: 185-215, A High-Resolution Multimode Digital Microscope System (Sluder & Wolf, Salmon), which discusses application software for automating a microscope and controlling a camera and the design of a hardware device comprising an automated optical microscope, a camera, and a Z-axis focusing device, which can be used herein. Image acquisition by a camera is described in detail in, for example, Inoue and Spring, Video Miroscopy, 2d. Edition, 1997, which is herein incorporated by reference. Real time display can also be performed using techniques well known in the art. For example, after all images are obtained and stored in a semi-permanent memory, or substantially at the same time as when an image is obtained, the image can be processed with appropriate application software to obtain processed data. For example, data may be processed by a method for playing back a sequence of images without interruption, a method for displaying images in real time, or a method for displaying images as a “movie” showing irradiating light as changes or continuation on a focal plane.

In another embodiment, application software for measurement and a presentation typically includes software for setting conditions for applying a stimuli or conditions for recording detected signals. With such a measurement and presentation application, a computer can have a means for applying a stimulus to cells and a means for processing signals detected from cells, and in addition, can control an optical observing means (a SIT camera and an image filing device) and/or a cell culturing means.

By inputting conditions for stimulation on a parameter setting screen using a keyboard, a touch panel, a mouse or the like, it is possible to set desired and complicated conditions for stimulation. In addition, various conditions, such as a temperature for cell culture, pH and the like, can be set using a keyboard, a mouse or the like. A display screen displays information on a network detected from a cell or information derived there from in real time or after recording. In addition, other recorded information or information derived there from of a cell can be displayed while being superimposed with a microscopic image of the cell. In addition to recorded information, measurement parameters in recording (stimulation conditions, recording conditions, display conditions, process conditions, various conditions for cells, temperature, pH, etc.) can be displayed in real time. The present invention may be equipped with a function of issuing an alarm when a temperature or pH departs from the tolerable range.

On a data analysis screen, in addition to the set theory as used in the present invention, it is possible to set conditions for various mathematical analyses, such as Fourier transformation, cluster analysis, FFT analysis, coherence analysis, correlation analysis and the like. The present invention may be equipped with a function of temporarily displaying information on a network, a function of displaying topography or the like. The results of these analyses can be displayed while being superimposed with the microscopic images stored in a recording medium.

### (Gene Introduction)

Any technique may be used herein for introduction of a nucleic acid molecule into cells, including, for example, transformation, transduction, transfection and the like. In the present invention transfection is preferable.

As used herein, the term “transfection” refers to an act of performing gene introduction or transfection by culturing cells with gene DNA, plasmid DNA, viral DNA, viral RNA or the like in a substantially naked form (excluding viral particles), or adding such a genetic material into cell suspension to allow the cells to take in the genetic material. A gene introduced by transfection is typically expressed within cells in a temporary manner or may be incorporated into the cells in a permanent manner.

Such a nucleic acid molecule introduction technique is well known in the art and commonly used, and is described in, for example, Ausubel F. A. et al., editors, (1988), Current Protocols in Molecular Biology, Wiley, New York, N.Y.; Sambrook J. et al. (1987) Molecular Cloning: A Laboratory Manual, 2nd Ed. and its 3rd Ed., Cold Spring Harbor Laboratory Press, Cold Spring Harbor, N.Y.; Special issue, Jikken Igaku [Experimental Medicine] “Experimental Methods for Gene introduction & Expression Analysis”, Yodo-sha, 1997; and the like. Gene introduction can be confirmed by methods as described herein, such as Northern blotting analysis and Western blotting analysis, or other well-known, common techniques.

When a gene is mentioned herein, the term “vector” or “recombinant vector” refers to a vector transferring a polynucleotide sequence of interest to a target cell. Such a vector is capable of self-replication or incorporation into a chromosome in a host cell (e.g., a prokaryotic cell, yeast, an animal cell, a plant cell, an insect cell, an individual animal, and an individual plant, etc.), and contains a promoter at a site suitable for transcription of a polynucleotide of the present invention. A vector suitable to perform cloning is referred to as a “cloning vector”. Such a cloning vector ordinarily contains a multiple cloning site containing a plurality of restriction sites. Restriction enzyme sites and multiple cloning sites, as described above, are well known in the art and can be used as appropriate by those skilled in the art depending on the purpose in accordance with publications described herein (e.g., Sambrook et al., supra).

As used herein, the term “expression vector” refers to a nucleic acid sequence comprising a structural gene and a promoter for regulating the expression thereof, and in addition, various regulatory elements in a state that allows them to operate within a host cells. The regulatory element may include, preferably, terminators, selectable markers such as drug-resistance genes and enhancers.

Examples of “recombinant vectors” for prokaryotic cells include, but are not limited to, pcDNA3(+), pBluescript-SK(+/−), pGEM-T, pEF-BOS, pEGFP, pHAT, pUC18, pFT-DEST™42GATEWAY (Invitrogen) and the like.

Examples of “recombinant vectors” for animal cells include, but are not limited to, pcDNAI/Amp, pcDNAI, pCDM8 (all commercially available from Funakoshi), pAGE107 [Japanese Laid-Open Publication No. 3-229 (Invitrogen), pAGE103 [J. Biochem., 101, 1307 (1987)], pAMo, pAMoA [J. Biol. Chem., 268, 22782-22787 (1993)], a retrovirus expression vector based on a murine stem cell virus (MSCV), PEF-BOS, pEGFP and the like.

Examples of a recombinant vectors for plant cells include, but are not limited to, pPCVICEn4HPT, pCGN1548, pCGN1549, pBI221, pBI121 and the like.

Any of the above-described methods for introducing DNA into cells can be used as a vector introduction method, including, for example, transfection, transduction, transformation, and the like (e.g., a calcium phosphate method, a liposome method, a DEAE dextran method, an electroporation method, a particle gun (gene gun) method and the like), a lipofection method, a spheroplast method (Proc. Natl. Acad. Sci. USA, 84, 1929 (1978)), a lithium acetate method (J. Bacteriol., 153, 163 (1983); and Proc. Natl. Acad. Sci. USA, 75, 1929 (1978)) and the like.

As used herein, the term “gene introduction reagent” refers to a reagent which is used in a gene introduction method so as to enhance the introduction efficiency. Examples of such a gene introduction reagent include, but are not limited to, cationic polymers, cationic lipids, polyamine-based reagents, polyimine-based reagents, calcium phosphate and the like. Specific examples of a reagent used in transfection include reagents available from various sources, such as and without limitation, Effectene Transfection Reagent (cat. no. 301425, Qiagen, CA), TransFast™ Transfection Reagent (E2431, Promega, WI), Tfx™-20 Reagent (E2391, Promega, WI), SuperFect Transfection Reagent (301305, Qiagen, CA), PolyFect Transfection Reagent (301105, Qiagen, CA), LipofectAMINE 2000 Reagent (11668-019, Invitrogen corporation, CA), JetPEI (×4) conc. (101-30, Polyplus-transfection, France) and ExGen 500 (R0511, Fermentas Inc., MD) and the like.

Gene expression (e.g., mRNA expression, polypeptide expression) may be “detected” or “quantified” by an appropriate method, including mRNA measurement and immunological measurement methods. Examples of molecular biological measurement methods include Northern blotting methods, dot blotting methods, PCR methods and the like. Examples of immunological measurement methods include ELISA methods, RIA methods, fluorescent antibody methods, Western blotting methods, immunohistological staining methods, and the like, where a microtiter plate may be used. Examples of quantification methods include ELISA methods, RIA methods and the like. A gene analysis method using an array (e.g., a DNA array, a protein array, etc.) may be used. The DNA array is widely reviewed in Saibo-Kogaku [Cell Engineering], special issue, “DNA Microarray and Up-to-date PCR Method”, edited by Shujun-sha. The protein array is described in detail in Nat Genet. 2002 December; 32 Suppl: 526-32. Examples of the methods for analyzing gene expression include, but are not limited to, RT-PCR methods, RACE methods, SSCP methods, immunoprecipitation methods, two-hybrid systems, in vitro translation methods, and the like in addition to the above-described techniques. Other analysis methods are described in, for example, “Genome Analysis Experimental Method, Yusuke Nakamura's Lab-Manual, edited by Yusuke Nakamura, Yodo-sha (2002), and the like. All of the above-described publications are herein incorporated by reference.

As used herein, the term “expression level” refers to the amount of a polypeptide or mRNA expressed in a subject cell. The term “expression level” includes the level of a protein expression of a polypeptide evaluated by any appropriate method using an antibody, including an immunological measurement methods (e.g., an ELISA method, an RIA method, a fluorescent antibody method, a Western blotting method, an immunohistological staining method and the like, or the mRNA level of expression of a polypeptide evaluated by any appropriate method, including molecular biological measurement methods (e.g., a Northern blotting method, a dot blotting method, a PCR method, and the like). The term “change in expression level” indicates that an increase or decrease in the protein or mRNA level of expression of a polypeptide evaluated by an appropriate method including the above-described immunological measurement method or a molecular biological measurement method.

### (RNAi)

As used herein, the term “RNAi” is an abbreviation of RNA interference and refers to a phenomenon where an agent for causing RNAi, such as double-stranded RNA (also called dsRNA), is introduced into cells and mRNA homologous thereto is specifically degraded, so that synthesis of gene products are suppressed, and a technique using the phenomenon. As used herein, RNAi may have the same meaning as that of an agent which causes RNAi.

As used herein, the term “an agent causing RNAi” refers to any agent capable of causing RNAi. As used herein, “an agent causing RNAi of a gene” indicates that the agent causes RNAi relating to the gene and the effect of RNAi is achieved (e.g., suppression of expression of the gene, and the like). Examples of such an agent causing RNAi include, but are not limited to, a sequence having at least about 70% homology to the nucleic acid sequence of a target gene or a sequence hybridizable under a stringent conditions, RNA containing a double-stranded portion having a length of at least 10 nucleotides or variants thereof. Here, this agent may be preferably DNA containing a 3′ protruding end, and more preferably the 3′ protruding end has a length of 2 or more nucleotides (e.g., 2-4 nucleotides in length).

Though not wishing to be bound by any theory, a mechanism which causes RNAi is considered to be as follows. When a molecule which causes RNAi, such as dsRNA, is introduced into a cell, an RNaseIII-like nuclease having a helicase domain (called dicer) cleaves the molecule on about a 20 base pair basis from the 3′ terminus in the presence of ATP in the case where the RNA is relatively long (e.g., 40 or more base pairs). As used herein, the term “siRNA” is an abbreviation of short interfering RNA and refers to short double-stranded RNA of 10 or more base pairs which are artificially chemically synthesized or biochemically synthesized, synthesized in the organism body, or produced by double-stranded RNA of about 40 or more base pairs being degraded within the organism. siRNA typically has a structure having 5′-phosphate and 3′-OH, where the 3′ terminus projects by about 2 bases. A specific protein is bound to siRNA to form RISC (RNA-induced-silencing-complex). This complex recognizes and binds to the mRNA having the same sequence as of a siRNA and cleaves the mRNA at the middle of a siRNA due to the RNaseIII-like enzymatic activity. It is preferable that the relationship between the sequence of siRNA and the sequence of the mRNA to be cleaved as a target is a 100% match. However, base mutations at a site away from the middle of the siRNA do not completely remove the cleavage activity by the RNAi, leaving partial activity, while base mutations in the middle of the siRNA have a large influence, and the mRNA cleavage activity by the RNAi is considerably lowered. By utilizing such a nature, only the mRNA having a mutation can be specifically degraded. Specifically, siRNA in which the mutation is provided in the middle thereof is synthesized and is introduced into a cell. Therefore, in the present invention, siRNA per se as well as an agent capable of producing siRNA (e.g., representatively dsRNA of about 40 or more base pairs) can be used as an agent capable of eliciting the RNAi.

Also, though not wishing to be bound by any theory, apart from the above-described pathway, the antisense strand of the siRNA binds to the mRNA such that the siRNA functions as a primer for an RNA-dependent RNA polymerase (RdRP), so that dsRNA is synthesized. This dsRNA is a substrate for a dicer, leading to production of new siRNA. It is intended that such an action is amplified. Therefore, in the present invention, siRNA per se and an agent capable of producing siRNA are useful. In fact, in insects and the like, for example, 35 dsRNA molecules can completely degrade 1,000 or more copies of intracellular mRNA, and therefore, it would be understood that siRNA per se as well as an agent capable of producing siRNA are useful.

In the present invention, double-stranded RNA having a length of about 20 bases (e.g., representatively about 21 to 23 bases) or less than about 20 bases, which is called siRNA, can be used. Expression of the siRNA in cells can suppress expression of a pathogenic gene targeted by the siRNA. Therefore, siRNA can be used for treatment, prophylaxis, prognosis, and the like of diseases.

The siRNA of the present invention may be in any form as long as it can elicit RNAi.

In another embodiment, an agent capable of causing RNAi may have a short hairpin structure having a sticky portion at the 3′ terminus (shRNA; short hairpin RNA). As used herein, the term “shRNA” refers to a molecule of about 20 or more base pairs in which a single-stranded RNA partially contains a palindromic base sequence and forms a double-strand structure therein (i.e., a hairpin structure). shRNA can be artificially chemically synthesized. Alternatively, shRNA can be produced by linking sense and antisense strands of a DNA sequence in reverse directions and synthesizing RNA in vitro with T7 RNA polymerase using the DNA as a template. Though not wishing to be bound by any theory, it should be understood that after the shRNA is introduced into a cell, the shRNA is degraded in the cell into a length of about 20 bases (e.g., representatively 21, 22, 23 bases) and causes RNAi in the same manner as the siRNA, leading to the treatment effect of the present invention. It should be understood that such an effect is exhibited in a wide range of organisms, such as insects, plants, animals (including mammals) and the like. Thus, shRNA elicits RNAi in the same manner as the siRNA and therefore can be used as an effective component of the present invention. shRNA may preferably have a 3′ protruding end. The length of the double-stranded portion is not particularly limited, but is preferably about 10 or more nucleotides, and more preferably about 20 or more nucleotides. Here, the 3′ protruding end may be preferably DNA, more preferably DNA of at least 2 nucleotides in length, and even more preferably DNA of 2-4 nucleotides in length.

An agent capable of causing RNAi used in the present invention may be artificially synthesized (chemically or biochemically) or naturally occurring. There is substantially no difference there between in terms of the effect of the present invention. A chemically synthesized agent is preferably purified by liquid chromatography or the like.

An agent capable of causing RNAi used in the present invention can be produced in vitro. In this synthesis system, T7 RNA polymerase and T7 promoter are used to synthesize antisense and sense RNAs from template DNA. These RNAs are annealed and thereafter are introduced into a cell. In this case, RNAi is caused via the above-described mechanism, thereby achieving the effect of the present invention. Here, for example, the introduction of an RNA into cell can be carried out by a calcium phosphate method.

Another example of an agent capable of causing RNAi according to the present invention is a single-stranded nucleic acid hybridizable to an mRNA or all nucleic acid analogs thereof. Such agents are useful for the method and composition of the present invention.

### (Calculation of Components)

As used herein the term “component” refers to any component necessary for phenotypic alteration of a living organism or a biological entity. As such, the component may be any biological agent. Therefore, a plurality of the components constitute a pathway. For example, it should be understood that such biological agents may include a nucleic acid, a protein, a gene in a broad sense (including miRNA and the like) and the like. In one embodiment, as the component, one derived from functional assay data using phenotype of a cell, tissue or an individual as an indicator. Components may be selected as a target calculated from a target collection of stimulants based on the functional assay data. Components may be a protein, or nucleic acid or both affecting the phenotype of interest, and preferably components may be selected by a functional assay from a limited number of candidate genes which may include the miRNA. Such a component may or may not be one known to be responsible for the phenotypic alteration. This is because the constitutive genes may also be the components.

As used herein, the term “component of interest” refers to a component for which an analysis is conducted.

As used herein, the term “reference component” refers to any component which an analysis makes reference to as a comparison. A component which is already known to take a certain value may be used. Alternatively, it should be noted that a component which takes a normal value, or the value thereof is unchanged may be used as the reference component.

As used herein, the term “upstream (component)” refers to a component which corresponds to the Preceding of a component of a pathway in which a plurality of the components have a precedent and succedent relationship.

As used herein, the term “downstream (component)” refers to a component which corresponds to the succeeding component of a pathway in which a plurality of the components have a precedent and succedent relationship.

It should be understood that those skilled in the art can specify a pathway of interest related to the phenotypic alteration and a reference pathway different from the pathway of interest, and specify a stimulant of interest and a reference stimulant which respectively stimulate the pathway of interest and the reference pathway by using any techniques of those known in the art or exemplified herein. For example, one can specify a stimulant which stimulates a pathway of interest by using a wet experimental technology. Alternatively, one can specify the stimulant by using in silico methods or a software employing a known database.

A collection of components of interest may be specified by giving a stimulant of interest to a living organism and observing and identifying the desired phenotypic alteration. In this manner, it is understood that any biological assay may be used to achieve this purpose.

Similarly, a collection of reference components may be specified by giving a reference stimulant to a living organism and observing and identifying the desired phenotypic alteration. In this manner, it is similarly understood that any biological assay may be used to achieve this purpose.

Intersection of the collection of components of interest and the collection of reference components may be calculated by using a set theory.

Differential collection between the collection of components of interest and the collection of reference components may be calculated by using a set theory. Furthermore, it is possible to determine if a component which belongs to the differential collection is determined to be present upstream or downstream of the intersection. Such a determination is described in WO 2006/046217 in detail, which is incorporated herein as a reference for its entirety.

### (Set Theory)

As mentioned above, the term “set theory” refers to a theory as used and understood in the art and the branch of pure mathematics, that deals with the nature and the relationships of sets. Many mathematicians use set theory as the basis for all other mathematics. Such set theory includes the analysis of objects (“elements or “members”) into sets (aggregates or collections) and classifying these sets into inclusion, independent and intersection and the like. Set theory is well known in the art and one skilled in the art can refer to Cantor, G., 1932, Gesammelte Abhandlungen, Berlin: Springer-Verlag; Ulam, S., 1930, ‘Zur Masstheorie in der allgemeinen Mengenlehre’, Fund. Math., 16, 140-150; Gödel, K., 1940, ‘The consistency of the axiom of choice and the generalized continuum hypothesis’, Ann. Math. Studies, 3; Scott, D., 1961, ‘Measurable cardinals and constructible sets’, Bull. Acad. Pol. Sci., 9, 521-524; Cohen, P., 1966, Set theory and the continuum hypothesis, New York: Benjamin; Jensen, R., 1972, ‘The fine structure of the constructible hierarchy’, Ann. Math. Logic, 4, 229-308; Martin, D. and Steel, J., 1989, ‘A proof of projective determinacy’, J. Amer. Math. Soc., 2, 71-125; Hrbacek, K. and Jech, T., 1999, Introduction to Set Theory, New York: Marcel Dekker, Inc, http://plato.stanford.edu/entries/set-theory/primer.html and the like, which is incorporated herein as a reference for its entirety.

The language of set theory is based on a single fundamental relation, called membership. As used herein, one may say that A is a member of B (in symbols A∈B), or that the set B contains A as its element. The understanding is that a set is determined by its elements. In other words, two sets are deemed equal if they have exactly the same elements. In practice, one considers sets of numbers, sets of points, sets of functions, sets of other sets and so on. In theory, it is not necessary to distinguish between objects. One only need to consider the sets.

Using the membership relation, one can derive other concepts usually associated with sets, such as unions and intersections of sets. For example, a set C is the union of two sets A and B if its members are exactly those objects that are either members of A or members of B. The set C is uniquely determined, because it has been specified what its elements are. There are more complicated operations on sets that can be defined in the language of set theory (i.e. using only the relation ∈), however these shall not be described here. Suppose another operation is mentioned: the (unordered) pair {A, B} has as its elements exactly the sets A and B. If it happens that A=B, then the “pair” has exactly one member, and is called a singleton {A}. By combining the operations of union and pairing, one can produce from any finite list of sets the set that contains these sets as members: {A, B, C, D, . . . , K, L, M}. The empty set, the set that has no elements, should also be mentioned. The empty set is uniquely determined by this property, as it is the only set that has no elements—this is a consequence of the understanding that sets are determined by their elements. When dealing with sets informally, such operations on sets are self-evident; with the axiomatic approach, it is postulated that such operations can be applied: for instance, one postulates that for any sets A and B, the set {A,B} exists. In order to endow set theory with sufficient expressive power one needs to postulate more general construction principles than those alluded to above. The guiding principle is that any objects that can be singled out can be collected into a set.

If a and b are sets, then the unordered pair {a, b} is a set whose elements are exactly a and b. The “order” in which a and b are put together plays no role; {a, b}={b, a}. For many applications, it is necessary to pair a and b in such a way that one can “read off” which set comes “first” and which comes “second.” It is denoted that this ordered pair of a and b by (a, b); a is the first coordinate of the pair (a, b), b is the second coordinate.

In an embodiment, the ordered pair has to be in a set. It should be defined in such way that two ordered pairs are equal if, and only if, their first coordinates are equal and their second coordinates are equal. This guarantees in particular that (a, b)≠(b, a) if a≠b.

Definition. (a, b)={{a}, {a, b}}.

If a≠b, (a, b) has two elements, a singleton {a} and an unordered pair {a, b}. The first coordinate can be found by looking at the element of {a}. The second coordinate is then the other element of {a, b}. If a=b, then (a, a)={{a}, {a,a}}={{a}} has only one element. In any case, it seems obvious that both coordinates can be uniquely “read off” from the set (a, b). This statement is made precise in the following theorem.

Theorem. (a, b)=(a′, b′) if and only if a=a′ and b=b′.

Proof. If a=a′ and b=b′, then, of course, (a, b)={{a}, {a, b}}={{a′}, {a′, b′}}=(a′,b′). The other implication is more intricate. Let us assume that {{a}, {a, b}}={{a′}, {a′, b′}}. If a≠b, {a}={a′} and {a, b}={a′, b′}. So, first, a=a′ and then {a, b}={a, b′} implies b=b′. If a=b, {{a}, {a, a}}={{a}}. So {a}={a′}, {a}={a′,b′}, and we get a=a′=b′, so a=a′ and b=b′ holds in this case, too.

With ordered pairs at our disposal, ordered triples can be defined:

(a, b, c)=((a, b), c),

ordered quadruples

(a, b, c, d)=((a, b, c), d),

and so on. Also, ordered “one-tuples” can be defined.

(a)=a.

A binary relation is determined by specifying all the ordered pairs of an objects in that relation; it does not matter by what property the set of these ordered pairs is described. Then the following definition is led.

Definition. A set R is a binary relation if all elements of R are ordered pairs, i.e., if for any z∈R there exist x and y such that z=(x, y).

As used herein according to the conventional art, it is conventional to describe xRy instead of (x, y)∈R. As used herein it is described that x is in relation R with y if xRy holds.

The set of all x which are in relation R with some y is called the domain of R and denoted by “dom R.” So dom R={x|there exists y such that xRy}. dom R is the set of all the first coordinates of ordered pairs in R.

The set of all y such that, for some x, x is in relation R with y is called the range of R, denoted by “ran R.” So ran R={y|there exists x such that xRy}.

Function, as understood in mathematics, is a procedure or a rule, assigning to any object, a, from the domain of the function a unique object, b, the value of the function at a. A function, therefore, represents a special type of relation, a relation where every object, a, from the domain is related to precisely one object in the range, namely, to the value of the function at a.

Definition. A binary relation F is called a function (or mapping, correspondence) if aFb1 and aFb2 imply b1=b2 for any a, b1, and b2. In other words, a binary relation F is a function if, and only if, for every a from dom F there is exactly one b such that aFb. This unique b is called the value of F at a and is denoted F(a) or Fa. [F(a) is not defined if a dom F.] If F is a function with dom F=A and ran F⊂B, it is customary to use the notations F: A B, <F(a)|a∈A>, <Fa|a ∈A>, <Fa>a∈A for the function F. The range of the function F can then be denoted {F(a)|a∈A} or {Fa}a ∈A.

The Axiom of Extensionality can be applied to functions as follows.

Lemma. Let F and G be functions. F=G if and only if dom F=dom G and F(x)=G(x) for all x∈dom F.

A function f is called one-to-one or injective if a1∈dom f, a2∈dom f, and a1≠a2 implies f(a1)≠f(a2). In other words if a1∈dom f, a 2 ∈dom f, and f(a1)=f(a2), then a1=a2.

In order to develop the mathematics within the framework of the axiomatic set theory, it is necessary to define natural numbers. Natural numbers are known intuitively: 0, 1, 2, 3, . . . , 15, . . . , 30, . . . , 115, . . . , 515, etc., and examples of sets having zero, one, two, or three elements can be easily given.

To define number 0, a representative of all sets having no elements is chosen. However, this is easy, since there is only one such set. 0=Ø is defined. Herein sets having one element (singletons) are defined: {Ø}, {{Ø}}, {{Ø, {Ø}}}; in general, {x}. A representative can be chosen as follows: Since one particular object has already been defined, namely 0, a natural choice is {0}. So it is defined:

1={0}={Ø}.

Next sets with two elements are considered: {Ø, {Ø}}, {{Ø}, {Ø, {Ø}}}, {{Ø}, {{Ø}}}, etc. By now, 0 and 1 have been defined, and 0≠1. A particular two-element set is singled out, the set whose elements are the previously defined numbers 0 and 1:

2={0,1}={Ø, {Ø}}.

It should begin to be obvious how the process continues:

3={0, 1, 2}={Ø, {Ø}, {Ø,{Ø}}}

4={0, 1, 2, 3}={Ø,{Ø}, {Ø,{Ø}}, {Ø, {0}, {Ø, {Ø}}}}

5={0, 1, 2, 3, 4}etc.

The idea is simply to define a natural number n as the set of all smaller natural numbers: {0, 1, . . . , n−1}. In this way, n is a particular set of n elements.

This idea still has a fundamental deficiency. 0, 1, 2, 3, 4, and 5 have been defined and could easily define 15 or 30 and not so easily 115 or 515. However, no list of such definitions teaches us what a natural number is in general. A statement of the form is necessary: “A set n is a natural number if . . . ” It cannot be simply determined that a set n is a natural number if its elements are all the smaller natural numbers, because such a “definition” would involve the very concept being defined.

The construction of the first few numbers is observed again. We defined 2={0, 1}. To get 3, it was necessary to adjoin a third element to 2, namely, 2 itself:

3=2∪{2}={0, 1}∪{2}.

Similarly,

4=3∪{3}={0, 1, 2}∪{3},

5=4∪{4}, etc.

Given a natural number n, the “next” number should be obtained by adjoining one more element to n, namely, n itself. The procedure works even for 1 and 2: 1=0∪{0}, 2=1∪{1}, but, of course, not for 0, the least natural number.

These considerations suggest the following.

Definition. The successor of a set x is the set S(x)=x∪{x}.

Intuitively, the successor S(n) of a natural number n is the “one bigger” number n+1. Herein the more suggestive notation n+1 for S(n) is used in what follows. Addition of natural numbers (using the notion of successor) can subsequently be defined in such a way that n+1 indeed equals the sum of n and 1. Until then, it is just a notation, and no properties of addition are assumed or implied by it.

The intuitive understanding of the natural numbers can now be summarized as follows:
1. 0 is a natural number.
2. If n is a natural number, then its successor n+1 is also a natural number.
3. All natural numbers are obtained by application of (a) and (b), i.e., by starting with 0 and repeatedly applying the successor operation: 0, 0+1=1, 1+1=2, 2+1=3, 3+1=4, 4+1=5, . . . etc.

Definition. A set I is called inductive if
1. 0∈1.
2. If n∈I, then (n+1)∈I.

An inductive set contains 0 and, with each element, also its successor. According to (c), an inductive set should contain all of the natural numbers. The precise meaning of (c) is that the set of the natural numbers is an inductive set which contains no other elements but the natural numbers, i.e., it is the smallest inductive set. This leads to the following definition.

Definition. The set of all natural numbers is the set

={x|x∈I for every inductive set I}.

The elements of the set are called natural numbers. Thus a set x is a natural number if, and only if, it belongs to every inductive set.

From the point of view of a pure set theory, the most basic question about a set is: “How many elements does it have?” It is a fundamental observation that the statement can be defined: “sets A and B have the same number of elements” without knowing anything about numbers.

Definition. Sets A and B have the same cardinality if there is a one-to-one function, f, with domain A and range B. This is be denoted by |A|=|B|.

Definition. The cardinality of A is less than or equal to the cardinality of B (notation: |A|≦|B|) if there is a one-to-one mapping of A onto B.

Notice that |A|≦|B| means that |A|=|C| for some subset C of B. |A|<|B| is also described to mean that |A|≦|B| and not |A|=|B|, i.e., that there is a one-to-one mapping of A onto a subset of B, but there is no one-to-one mapping of A onto B.

Lemma.
1. If |A|≦|B| and |A|=|C|, then |C|≦|B|.
2. If |A|≦|B| and |B|=|C|, then |A|≦|C|.
3. |A|≦|A|.
4. If |A|≦|B| and |B|≦|C|, then |A|≦|C|.

Cantor-Bernstein Theorem. If |X|≦|Y| and |Y|≦|X|, then |X|=|Y|.

Finite sets can be defined as those sets whose size is a natural number.

Definition. A set, S, is finite if it has the same cardinality as some natural number, n. Then, |S|=n is defined and it can be described that S has n elements. A set is infinite if it is not finite.

As described above, set theory can be applied to a n analysis of the present invention.

For example, a test result can be summarized in an Excel(R)-format file, in which functional reporters such as a transcriptional factor reporters, and perturbation agents such as a siRNA's are plotted in an x-y format, and the value corresponding to each combination thereof is filled therein. The actual value may be compared to a standard value, or a threshold of interest such as a result obtained by using a scrambled siRNA. The values may be normalized into three values such as +, 0 and −. The values are evaluated, for example, when 80% or less of the threshold, it is normalized to “−1”, and when between 80% and 120% of the threshold, it is normalized to “0”, and when 120% or more of the threshold, it is normalized to “+1”. The normalized or degenerated matrix may be used to analyze the effects of perturbation agents (such as siRNA's) on reporters in a simpler manner, and to obtain a set of perturbation agents giving an effects on each of the reporters. An exemplary table is shown below.

| | | | |
|---|---|---|---|
| Function 1 | Function 2 | Function 3 | Function 4 |

before normalization

| | | | | |
|---|---|---|---|---|
| siRNA 1 | 70% | 120% | 80% | 75% |
| siRNA 2 | 115% | 100% | 65% | 130% |
| siRNA 3 | 150% | 90% | 105% | 115% |

after normalization

| | | | | |
|---|---|---|---|---|
| siRNA 1 | −1 | +1 | −1 | −1 |
| siRNA 2 | 0 | 0 | −1 | +1 |
| siRNA 3 | +1 | 0 | 0 | 0 |

### DESCRIPTION OF PREFERRED EMBODIMENTS

Hereinafter, the present invention will be described by way of embodiments. Embodiments described below are provided only for illustrative purposes. Accordingly, the scope of the present invention is not limited by the embodiments except as by the appended claims.

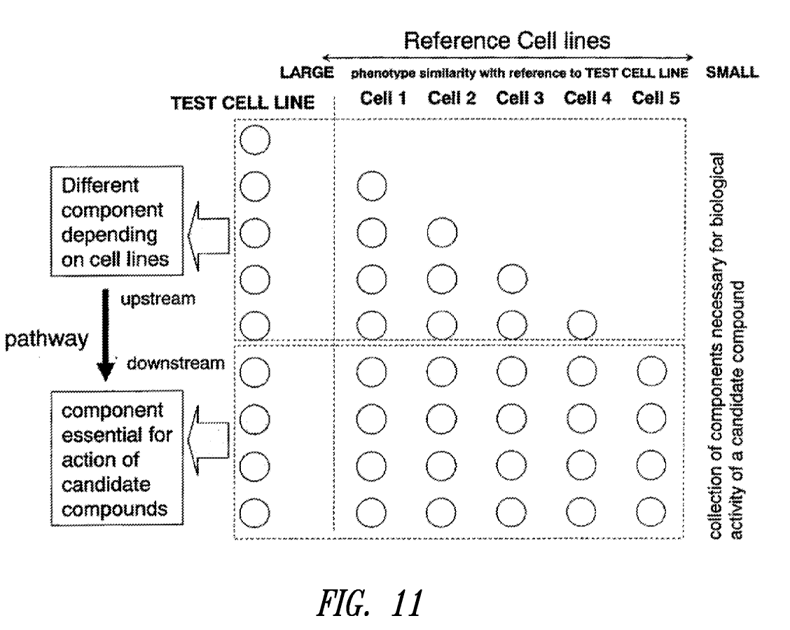

As shown in FIG. 11, which shows the concept of the present invention, reactivity of compounds to cell species for testing and reference cell species (such as cells 1, 2, 3, 4 and 5, which have larger to smaller similarities to the test cell species) are analyzed and the component collections necessary for the biological activity of a compound are determined. Thereafter, components different from cell species to cell species are determined from upstream to downstream by means of the present method of the present invention and the component essential for the action of the component is determined.

In one aspect, the present invention provides a method for deriving an upstream or downstream component of a component necessary for a phenotypic alteration of a living organism, the method comprising the steps of: A) specifying a pathway of interest related to the phenotypic alteration and a reference pathway different from the pathway of interest, and specifying a stimulant of interest and a reference stimulant which respectively stimulate the pathway of interest and the reference pathway; B) giving the stimulant of interest to the living organism to identify a collection of the components of interest necessary for the phenotypic alteration; C) giving the reference stimulant to the living organism to identify a collection of the reference components necessary for the phenotypic alteration; D) calculating an intersection between the collection of the components of interest and the reference components; and E) calculating differential collection by subtracting the intersection from the collection of components of interest, wherein a component which belongs to the differential collection is determined to be present upstream or downstream of the intersection.

The step of subjecting a biological entity to a stimulant may be conducted in any manner as long as the perturbation agent is conducted to the entity and attains the effects of interest, and is dependent on the type of stimulant used. So long as set theory can be conducted, the step of subjecting the data to set theory can be conducted. Preferably, a biological entity used in the present invention is a cell.

Stimulants used in the present invention may be any agents which give a perturbation or a change to a biological entity or a system such as an RNA including siRNA, shRNA, miRNA, and ribozyme, chemical compound, cDNA, antibody, polypeptides, light, sound, pressure change, radiation, heat, gas and the like. Preferably a siRNA capable of specifically regulating a function of a said functional reporter. Functional reporters used in the present invention include but are not limited to transcriptional factors, regulatory genes, structural genes, cellular markers, cell surface markers, cell shapes, organelle shapes, cell mobility, enzyme activities, metabolite concentrations, and localization of cellular components.

In a specific embodiment, the set theory processing used in the present invention may be conducted by classifying two specific functional reporters of at least two said functional reporters into a relationship selected from the group consisting of a) independent, b) inclusion, and c) intersection, wherein when it is determined to be independent, the two specific functional reporters are determined to have no relationships in the network; when it is determined to be inclusive, one of the two specific functional reporters is determined to be included in the other of the two specific functional reporters and located downstream of the other; when it is determined to be intersection, the two specific functional reporters are determined to be located downstream branched from another common function.

In the present invention, any mathematical process of a set theory can be used as long as sets can be analyzed according to the set theory. In a specific embodiment of the present invention, the set theory processing comprises the step of mapping the absence or presence of a response by said stimulant per said functional reporter. In a specific embodiment of the present invention, the set theory processing can comprise a calculation of a relationship between the reporters comprising correlation between each functional reporter as classified into an independent, inclusion and intersection to generate a summary of the correlation. This calculation can be conducted by using a matrix. In a specific embodiment, the effect obtained by the stimulants used can be classified into the following three groups in terms of a threshold value: positive effect=+(preferably +1); no effect=0; and negative effect=−(preferably −1).

In one embodiment, the living organism or biological entity is a cell. In another embodiment, the living organism is grown under two or more different conditions.

In a preferable embodiment, the component is induced from functional assay data which is an indication of a cell, tissue or an individual.

In another embodiment, the component is selected based on a functional assay from a limited number of candidate genes including a miRNA.

In another embodiment, the component is a target calculated based on functional assay data from the target collection of the stimulant.

In another embodiment, the component is a protein, a nucleic acid or both, which has an effect on a phenotype of interest.

In another embodiment, the component is derived from the result of a functional screening from a limited number of the functional nucleic acid libraries.

In another embodiment, the stimulant is an antibody, an RNA interference agent or a molecular target inhibitor.

In a preferable embodiment of the present invention, the information on at least two functional reporters is based on an effect of the stimulant after a desired time; wherein the set theory processing comprises: a) classifying the information into three categories by comparing the effect with a threshold value for the functional reporter and classifying them into the following three groups: positive effect=+(preferably +1); no effect=0; and negative effect=−(preferably −1); b) determining if two out of the functional reporters have a common stimulant, wherein the common stimulant has the same type of effect, and if there are no such common stimulants, then the two functions corresponding to the two functional reporters are located under different stimulants, and if there is such a common stimulant, then the following step c) is conducted: c) determining if the stimulant set for one function of the two functions is completely included into the stimulant set for the other function of the two functions, and if this is the case, then one function having the bigger set is located downstream of the other function having the smaller set, and if this is not the case, then the two functions are located in parallel under the same stimulants; d) determining if all the combinations of the functional reporters are investigated, if this is the case, then integrating all the relationships of the functions to present a global perturbation effects network, and if this is not the case then repeating the steps a) to c). These steps can be conducted on a computer equipped with a computer program implementing the process and steps of interest.

In a further embodiment of the present invention, the present invention may further comprise analyzing the generated network by conducting an actual biological experiment. Preferably, such an analysis comprises the use of a regulation agent such as siRNA, antibody, antisense oligonucleotide, inhibitor, activator, ligand, receptors and the like, specific to the function. Preferably, siRNA is used.

The present invention can be used for analyzing networks such as a signal transduction pathway, a cellular pathway and the like.

The present invention is useful for identification of a biomarker, analysis of a drug target, analysis of a side effect, diagnosis of a cellular function, analysis of a cellular pathway, evaluation of a biological effect of a compound, and diagnosis of an infectious disease and the like.

In another aspect, the present invention provides a method for profiling a compound comprising the step of repeatedly applying the method of the present invention. As used herein, any preferable or other embodiments may be used for the subject method for profiling a compound.

In another aspect, the present invention provides a process for profiling a compound which can be combined for a use in achieving the phenotypic alteration, the process comprising the step of repeatedly applying the method according to claim 1, wherein the process further comprises the steps of: A) calculating the collection of components of interest which increases phenotypic alteration expected by a living organism under a culture condition in which the compound is added; B) calculating the collection of components of interest which increases phenotypic alteration expected by a living organism under a culture condition in which there is no compound; C) calculating a differential collection of the collection of components of interest and the collection of reference components thereby calculating a specific component collection appearing under the culture conditions with a compound added thereto; D) calculating a common pathway of components included in the specific component collection; and E) selecting a compound which targets the common pathway. As used herein, any preferable or other embodiments may be used for the subject method for profiling a compound.

In another aspect, the present invention provides a process for searching for a target of a compound, comprising the method according to claim 1, the process further comprising the steps of: A) calculating the collection of components of interest which increases phenotypic alteration expected by a living organism under a culture condition in which the compound which can be combined for use in achieving the phenotypic alteration is added; B) calculating the collection of components of interest which increases phenotypic alteration expected by a living organism under a culture condition in which there is no compound which can be combined for use in achieving the phenotypic alteration; C) calculating a differential collection of the collection of components of interest and the collection of reference components, thereby calculating a specific component collection appearing under the culture conditions with a compound added thereto; D) calculating a common pathway of components included in the specific component collection; and E) selecting a target included in the common pathway. As used herein, any preferable or other embodiments may be used for the subject method for profiling a compound.

In another aspect, the present invention provides a process for searching for a target of a similar compound, comprising the method according to claim 1, wherein the process comprises the steps of: A) calculating the collection of the components of interest which increases phenotypic alteration expected by a living organism in a culture circumstance in which the compound which can be combined for the use in achieving the phenotypic alteration is added; B) calculating the collection of the components of interest which increases phenotypic alteration expected by a living organism under a culture condition in which there are no compounds which can be combined for use in achieving the phenotypic alteration; C) calculating a differential collection of the collection of the components of interest and the collection of the reference components, thereby calculating a specific component collection appearing under the culture condition with a compound added thereto; D) calculating a common pathway of the components included in the specific component collection; and E) selecting a target of a similar compound from the common pathway. As used herein, any preferable or other embodiments may be used for the subject method for profiling a compound.

In a preferable aspect of the invention, the present invention provides a method for inhibiting breast cancer using a combination of DXR and at least an inhibitor of the EphA family. Examples of an inhibitor of the EphA family include RNAi molecules thereof.

In a preferable aspect of the invention, the present invention provides a method for inhibiting breast cancer using a combination of DXR and at least an inhibitor of the EphB family. Examples of an inhibitor of the EphB family include RNAi molecules thereof.

In a preferable aspect of the invention, the present invention provides a method for inhibiting breast cancer using a combination of DXR and at least an inhibitor of c-KIT. Examples of an inhibitor of the c-KIT family include RNAi molecules thereof.

In a preferable aspect of the invention, the present invention provides a method for inhibiting breast cancer using a combination of DXR and at least an inhibitor of ALK. Examples of an inhibitor of the ALK family include RNAi molecules thereof.

In a preferable aspect of the invention, the present invention provides a system for deriving an upstream or downstream component necessary for phenotypic alteration of a living organism, the system comprising: A) a computer for specifying a pathway of interest related to the phenotypic alteration and a reference pathway different from the pathway of interest, and specifying a stimulant of interest and a reference stimulant which respectively stimulate the pathway of interest and the reference pathway; B) an assay system for giving the stimulant of interest to the living organism to identify a collection of components of interest necessary for the phenotypic alteration; C) an assay system for giving the reference stimulant to the living organism to identify a collection of reference components necessary for the phenotypic alteration; D) a computer for calculating an intersection between the collection of the components of interest and the reference components; and E) a computer for calculating differential collection by subtracting the intersection from the collection of components of interest, wherein a component which belongs to the differential collection is determined to be present upstream of or downstream the intersection. As used herein, any preferable or other embodiments may be used for the subject method for profiling a compound.

Means for obtaining information on at least two functional reporters in the said biological entity, wherein the functional reporters reflect a biological function, may be provided as a transfection array, but the present invention is not limited to this. Such a transfection array is extensively described elsewhere herein and exemplified in the following Examples.

Means for subjecting the obtained information to set theory processing to calculate a relationship between the functional reporters to generate a network relationship of the biological functions, may be provided as a computer program but the present invention is not limited to this. As a set theory is known in the art, it is understood that any computer program implementing such a calculation based on set theory can be used in the present invention.

In a preferable aspect of the invention, the present invention provides a program for implementation by a computer to conduct a method for deriving an upstream or downstream component necessary for phenotypic alteration of a living organism, the method comprising the steps of: A) specifying a pathway of interest related to the phenotypic alteration and a reference pathway different from the pathway of interest, and specifying a stimulant of interest and a reference stimulant which respectively stimulate the pathway of interest and the reference pathway; B) giving the stimulant of interest to the living organism to identify a collection of components of interest necessary for the phenotypic alteration; C) giving the reference stimulant to the living organism to identify a collection of reference components necessary for the phenotypic alteration; D) calculating an intersection between the collection of the components of interest and the reference components; and E) calculating a differential collection by subtracting the intersection from the collection of components of interest, wherein a component which belongs to the differential collection is determined to be present upstream or downstream of the intersection. As used herein, any preferable or other embodiments may be used for the subject method for profiling a compound.

It should be noted that those skilled in the art will understand that any other specific embodiments of the method and system as described hereinabove may be employed and are applicable to a computer program of the present invention if necessary.

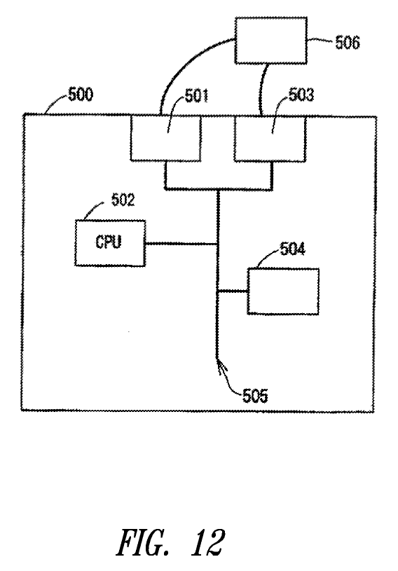

A configuration of a computer or system for implementing a method of the present invention for analyzing a network of a biological functions in a biological entity is shown in FIG. 12. FIG. 12 shows an exemplary configuration of a computer 500 for executing the compound profiling method of the present invention.

The computer 500 comprises an input section 501, a CPU 502, an output section 503, a memory 504, and a bus 505. The input section 501, the CPU 502, the output section 503, and the memory 504 are connected via a bus 505. The input section 501 and the output section 503 are connected to an I/O device 506.

An outline of a process for presenting a state of a cell, which is executed by the computer 500, will be described below.

A program for executing a method for analyzing a network of biological functions in a biological entity is stored in, for example, the memory 502. Alternatively, information necessary for the method may be stored in any type of recording medium, such as a floppy disk, MO, CD-ROM, CD-R, DVD-ROM, or the like separately or together. Alternatively, the program may be stored in an application server. The information or data stored in such a recording medium is loaded via the I/O device 506 (e.g., a disk drive, a network (e.g., the Internet)) to the memory 504 of the computer 500. The CPU 502 executes the cellular state presenting the program, so that the computer 500 functions as a device for performing a method of the present invention for analyzing a network of biological functions in a biological entity.

Information about a cell or the like is input via the input section 501 as well as data obtained. Known information may be input as appropriate.

The CPU 502 generates display data based on the information about data and cells through the input section 501, and store the display data into the memory 504. Thereafter, the CPU 502 may store the information in the memory 504. Thereafter, the output section 503 outputs a network analyzed by the CPU 502 as display data. The output data is output through the I/O device 506.

In a preferable aspect of the invention, the present invention provides a storage medium with a program stored thereon for implementation by a computer to conduct a method for deriving upstream or downstream of a component necessary for phenotypic alteration of a living organism, the method comprising the steps of: A) specifying a pathway of interest related to the phenotypic alteration and a reference pathway different from the pathway of interest, and specifying a stimulant of interest and a reference stimulant which respectively stimulate the pathway of interest and the reference pathway; B) giving the stimulant of interest to the living organism to identify a collection of components of interest necessary for the phenotypic alteration; C) giving the reference stimulant to the living organism to identify a collection of reference components necessary for the phenotypic alteration; D) calculating an intersection between the collection of the components of interest and the reference components; and E) calculating a differential collection by subtracting the intersection from the collection of the components of interest, wherein a component which belongs to the differential collection is determined to be present upstream or downstream of the intersection. As used herein, any preferable or other embodiments may be used for the subject method for profiling a compound.

It should be noted that those skilled in the art will understand that any other specific embodiments of the method, system and computer program as described hereinabove may be employed and are applicable to a storage medium of the present invention if necessary. Such a storage medium may be any type of recording medium, such as CD-ROMs, flexible disks, CD-Rs, CD-RWs, MOs, mini disks, DVD-ROMs, DVD-Rs, memory sticks, hard disks, and the like.

In a preferable aspect of the invention, the present invention provides a composition for inhibiting breast cancer comprising a combination of DXR and at least an inhibitor of the EphA family. Examples of an inhibitor of the EphA family includes RNAi molecules thereof.

In a preferable aspect of the invention, the present invention provides a composition for inhibiting breast cancer comprising a combination of DXR and at least an inhibitor of the EphB family. Examples of an inhibitor of the EphB family includes RNAi molecules thereof.

In a preferable aspect of the invention, the present invention provides a composition for inhibiting breast cancer comprising a combination of DXR and at least an inhibitor of c-KIT. Examples of an inhibitor of the c-KIT family include RNAi molecules thereof.

In a preferable aspect of the invention, the present invention provides a composition for inhibiting breast cancer comprising a combination of DXR and at least an inhibitor of ALK. Examples of an inhibitor of the ALK family include RNAi molecules thereof.

Eph family: The family involves 14 receptor tyrosine kinases. The family is subdivided into two classes: EphA (SEQ ID NOs: 1-12) and EphB (SEQ ID NOs: 13-20). The family is expressed in the nervous systems during development and in adult. The family is considered as responsible for the process of axon guidance. The family is also expressed in breast cancer cells. The specific inhibitors against of this family are unknown.

C-Kit: The stem cell factor-c-kit is a receptor tyrosin kinase previously implicated in the hematopoietic recovery (e.g. Homo sapiens v-kit Hardy-Zuckerman 4 feline sarcoma viral oncogene homolog (KIT) (SEQ ID NO: 21-22)). The tyrosine kinase is expressed in several cancers. An anti-cancer drug “imatinib” is one of the famous c-kit inhibitors.

ALK: ALK is a receptor tyrosine kinase (e.g. Homo sapiens v-kit Hardy-Zuckerman 4 feline sarcoma viral oncogene homolog (KIT) (SEQ ID NO: 23-24)). The protein is responsible for development of nerve systems. The molecular mechanisms are unknown. The specific inhibitors are still unknown.

It should be noted that those skilled in the art will understand that any other specific embodiments of the method, system, computer program and storage medium as described hereinabove may be employed and are applicable to a transmission medium of the present invention if necessary. Examples of such a transmission medium include, but are not limited to, networks, such as intranets, the Internet and the like.

The preferred embodiments of the present invention have been heretofore described for a better understanding of the present invention. Hereinafter, the present invention will be described by way of examples. Examples described below are provided only for illustrative purposes. Accordingly, the scope of the present invention is not limited except as by the appended claims. According to the examples below, it will be understood that those skilled in the art can select cells, supports, biological factors, salts, positively charged substances, negatively charged substances, actin acting substances and the like, as appropriate, and can make or carry out the present invention.

### EXAMPLES

Hereinafter, the present invention will be described in greater detail by way of examples, though the present invention is not limited to the examples below. Reagents, supports, and the like were commercially available from Sigma (St. Louis, USA), Wako Pure Chemical Industries (Osaka, Japan), Matsunami Glass (Kishiwada, Japan) unless otherwise specified.

### Example 1

### Pathway in which Projection Extension from Neurological Precursor Cells

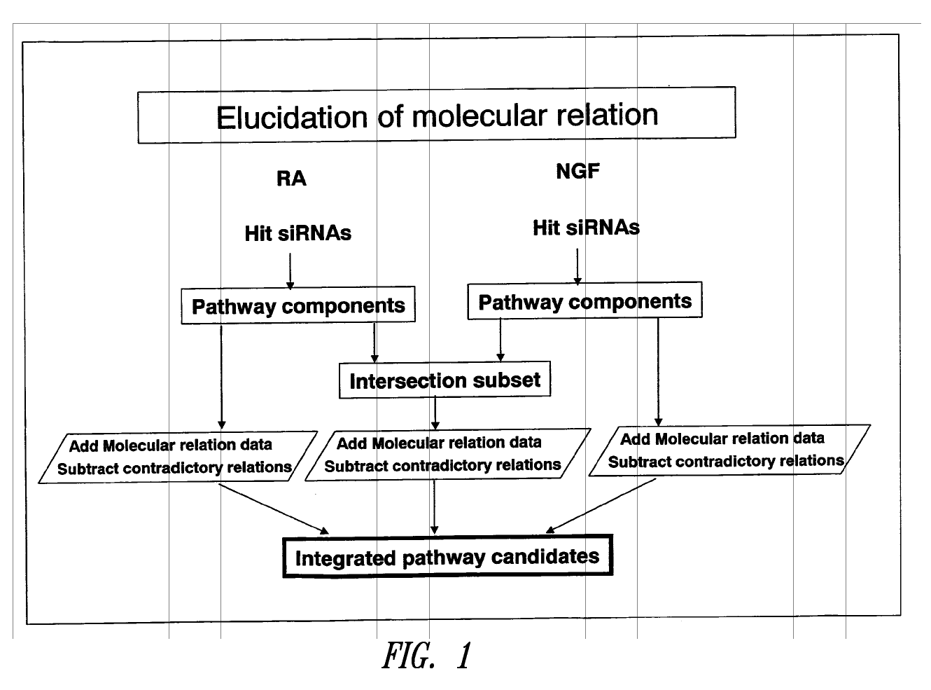

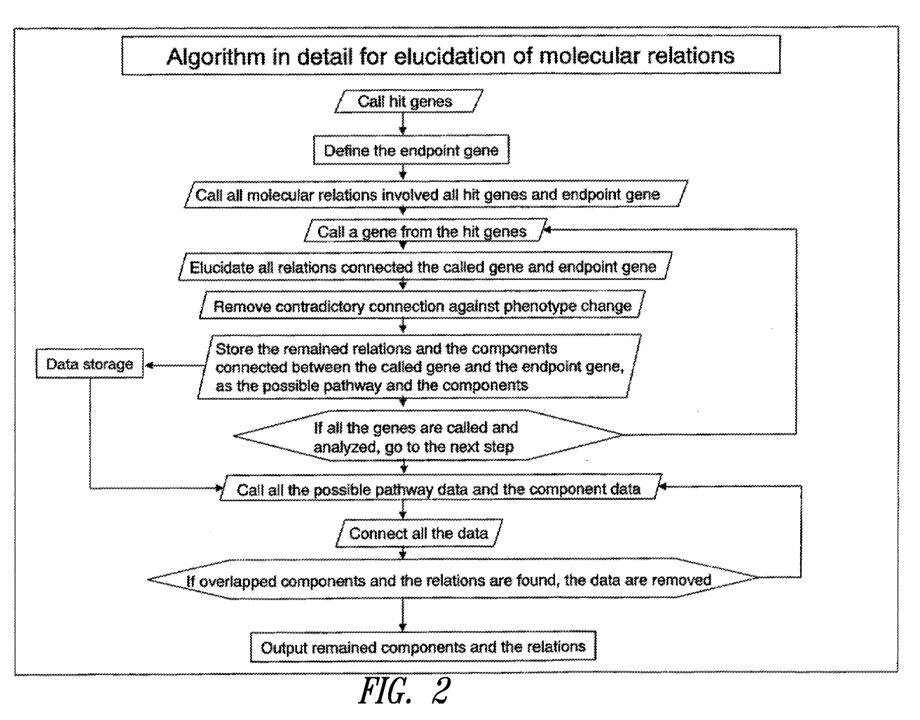

As a specific example, pathway in which projection extension from neurological precursor cells was tested and is described below. Methodologies employed herein have been illustrated in FIGS. 1 and 2.

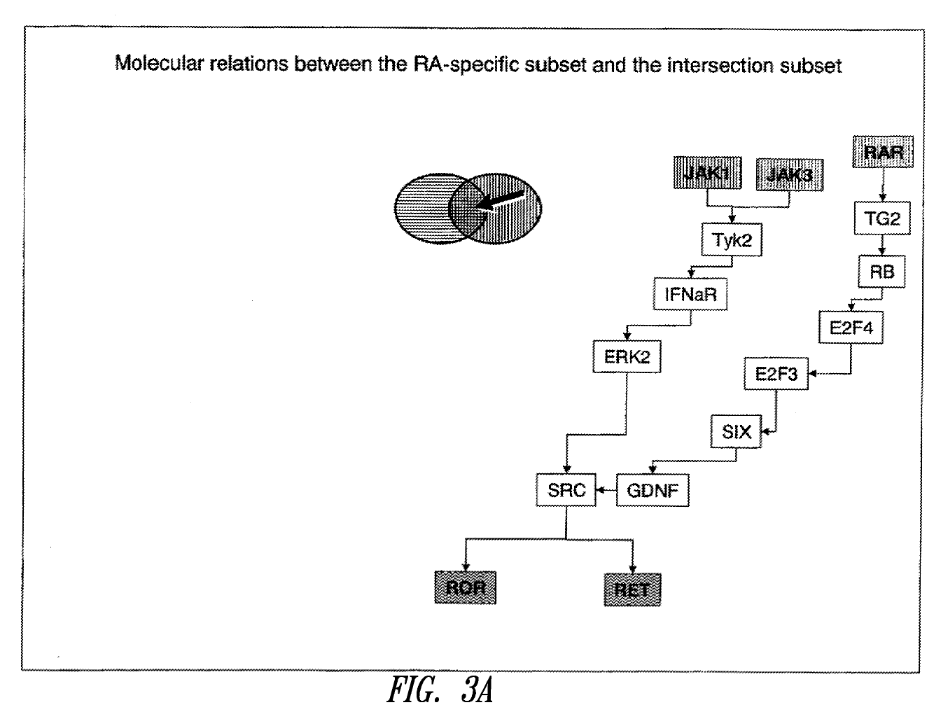

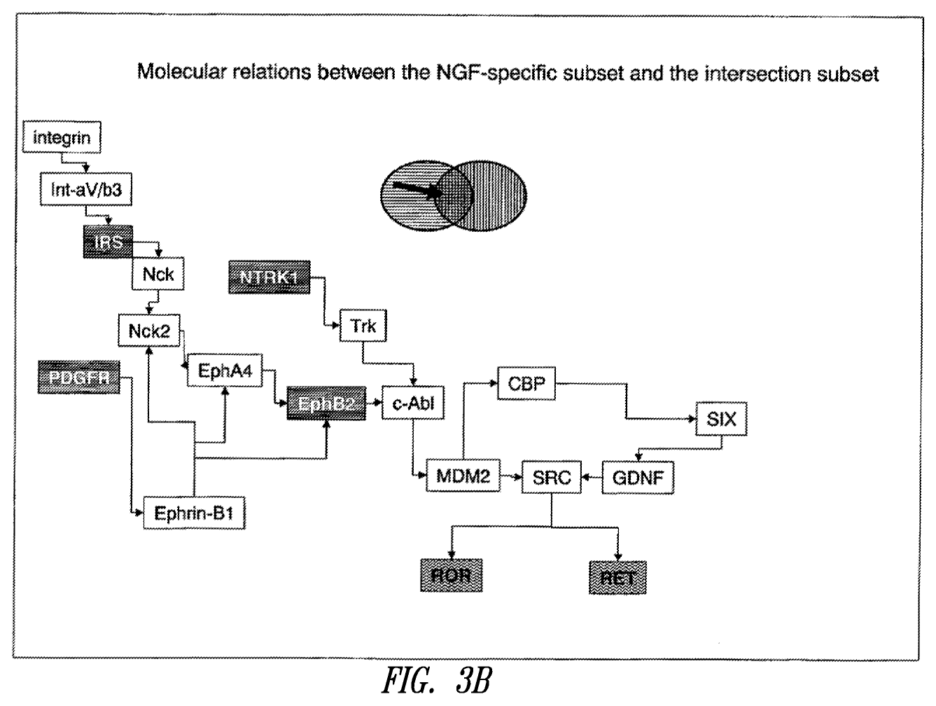

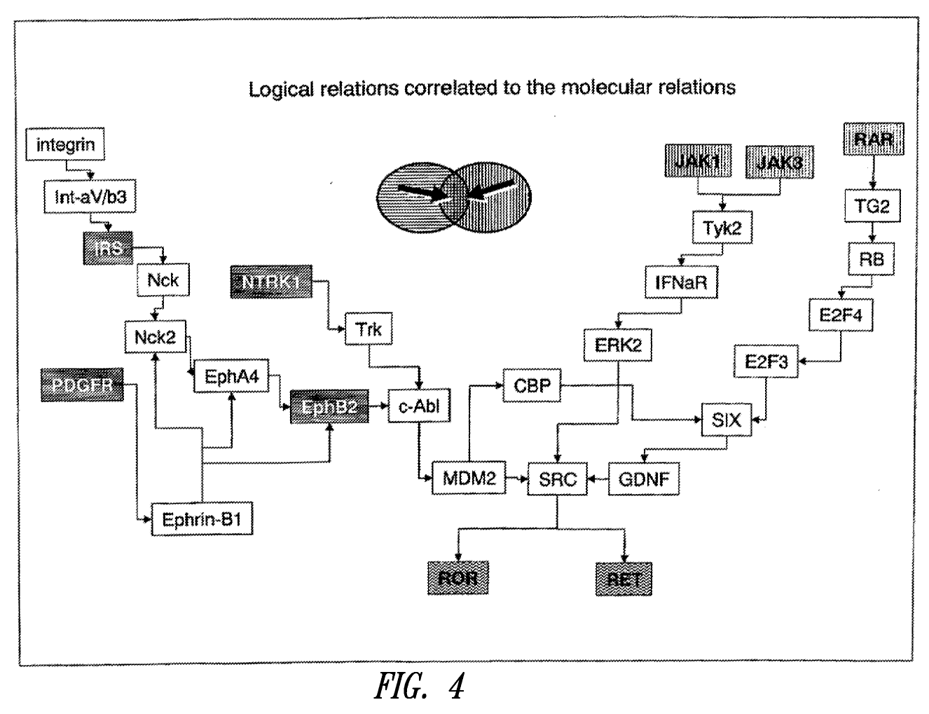

Retinoic acid was exposed to SHSY5Y cell, a neuroblastoma from human to raise expression of choline acetyl transferase and cause extension of neuron projection, and forms cholinergic neuron cells (1). On the other hand, when Nerve Growth Factor (NGF) was exposed to the same cell, tyrosine hydroxylase was expressed and caused extension of neuron projection, and forms dopaminergic neuron cells (2). The functions of (1) and (2) are different, but share the same properties in terms of extension of neuron projection. Therefore, cellular components necessary for these two intracellular alteration were specified experimentally by means of RNA interference method. As such, it was obtained that the component collection (3) necessary for the alteration from SHSY5Y to (1) are {JAK1, JAK3, ROR, and RET}. On the other hand, the component collection (4) necessary for the alteration from SHSY5Y cells to (2), are {NTRK1, EPHB2, INSR, RDGFRA, ROR and RET}. When comparing and calculation of intersection is done for (3) and (4), the intersection (5) was obtained to read {ROR and RET}. Next, the collection (6) {JAK1 and JAK3} by subtraction of (3)−(5) and the collection (5) were analyzed in terms of an intermolecular interaction, and the existence of a pathway of a molecular signals from (6) to (5) (FIG. 3A) was determined. Furthermore, the collection (7) which was obtained by the subtraction of (4)−(5), and the collection (5) were analyzed in terms of an intermolecular interaction, and the existence of a pathway of molecular signals from (7) to (5) (FIG. 3B) was further determined. The common components and pathway relating to the projection extension by retinoic acid (RA) and Nerve Growth Factor (NGF) are shown in FIG. 4. As seen from FIG. 4, The output graph indicates molecular relations between the hit molecules (RAR, JAK1, and JAK3) for retinoic acid (RA), the hit molecules (IRS, PDGFR, NTRK1, and RPHB2) for Nerve Growth Factor (NGF), and the endpoint molecules (ROR and RET). As such, it was demonstrated that the collection structure reflects the upstream and downstream of a pathway.

### Example 2

### Pathway Analysis of Human Derived Breast Cancer Cells

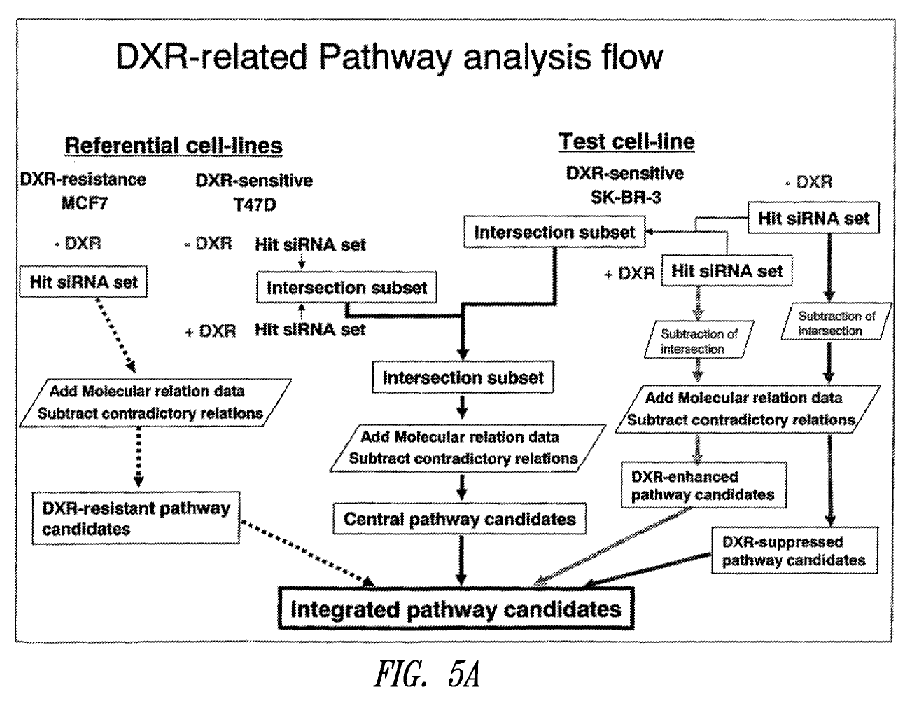

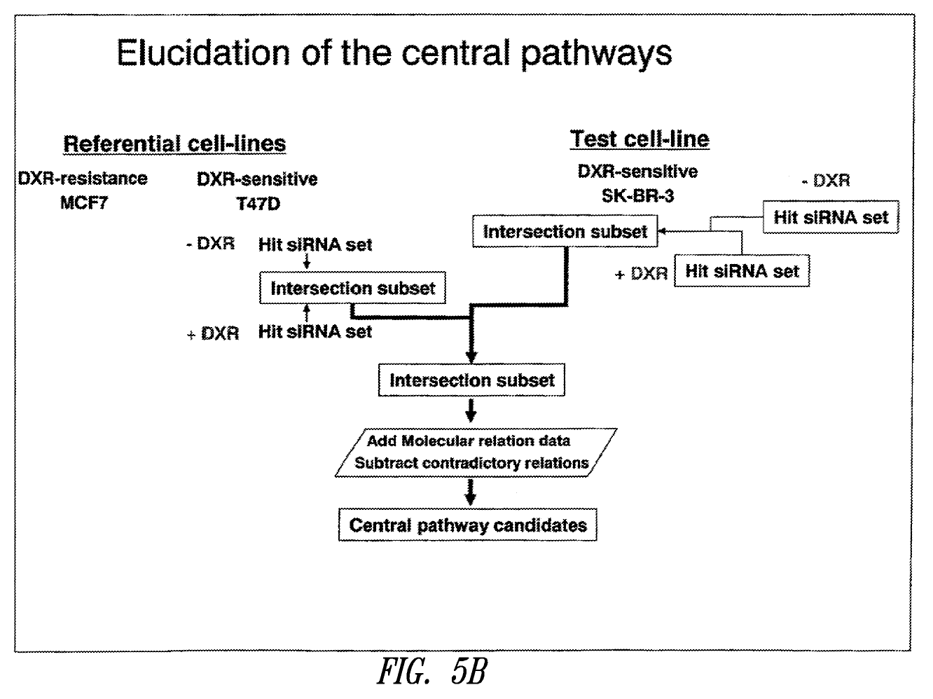

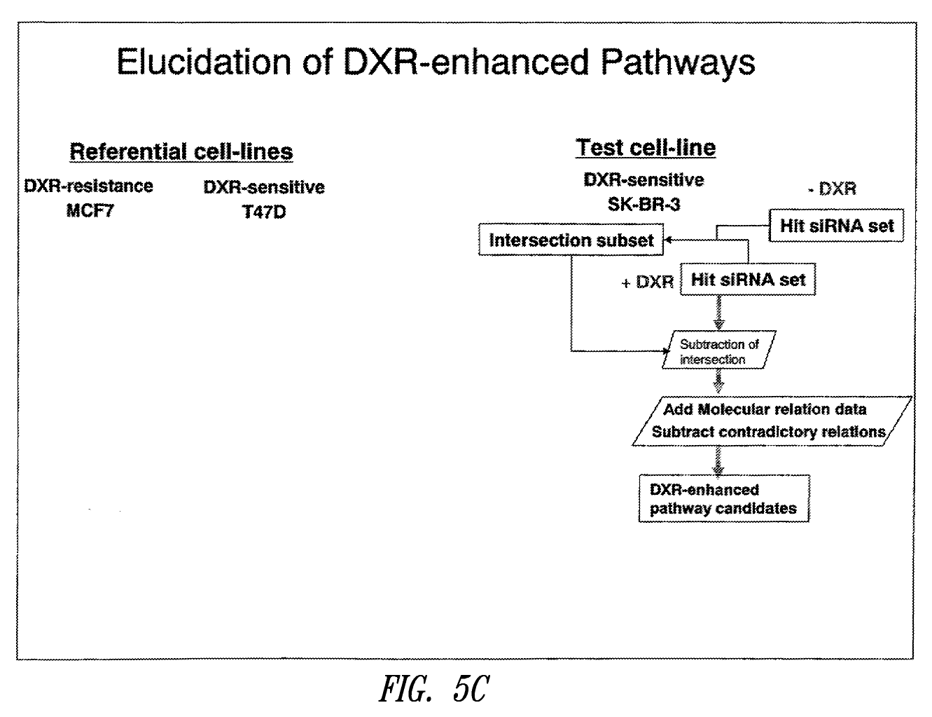

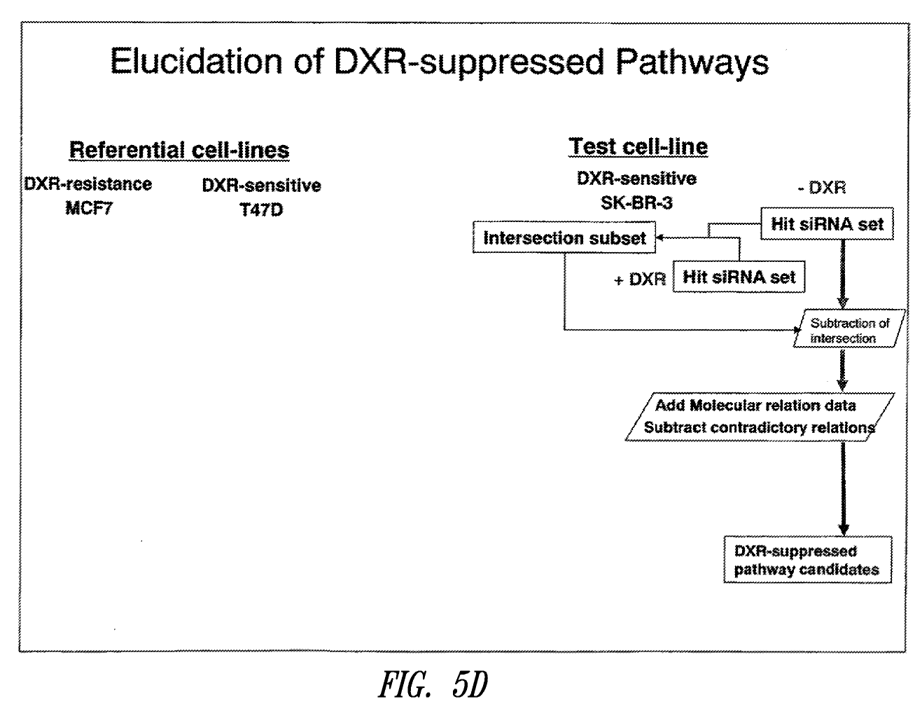

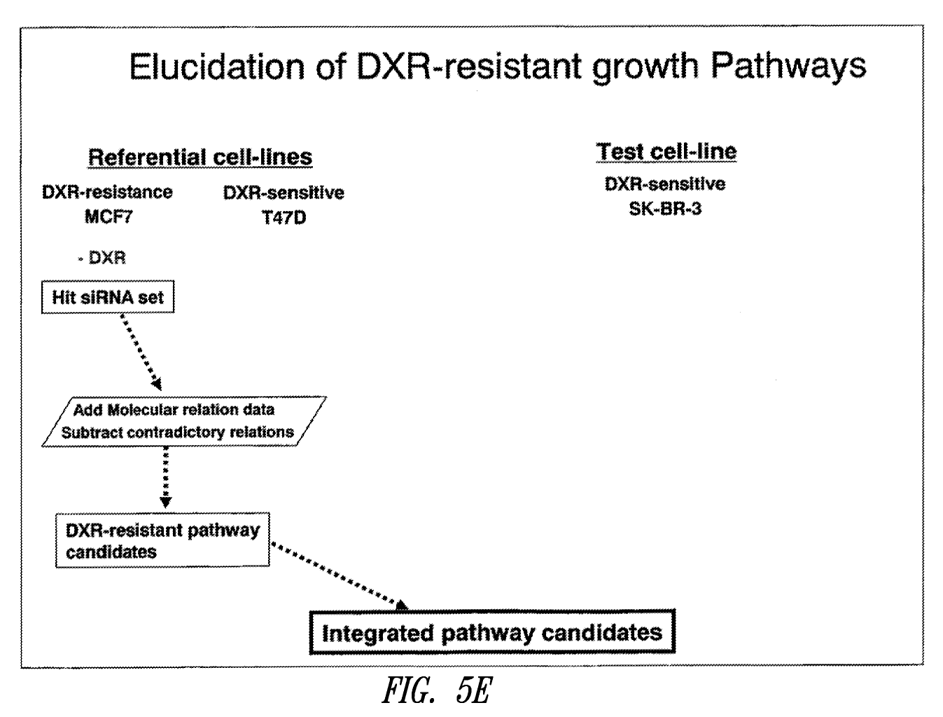

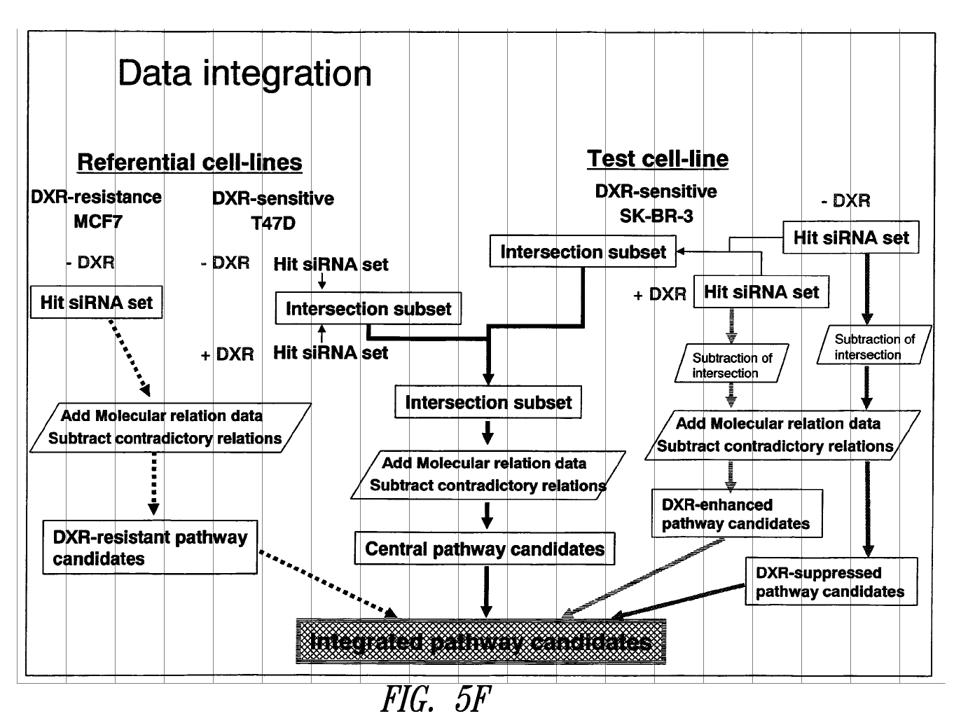

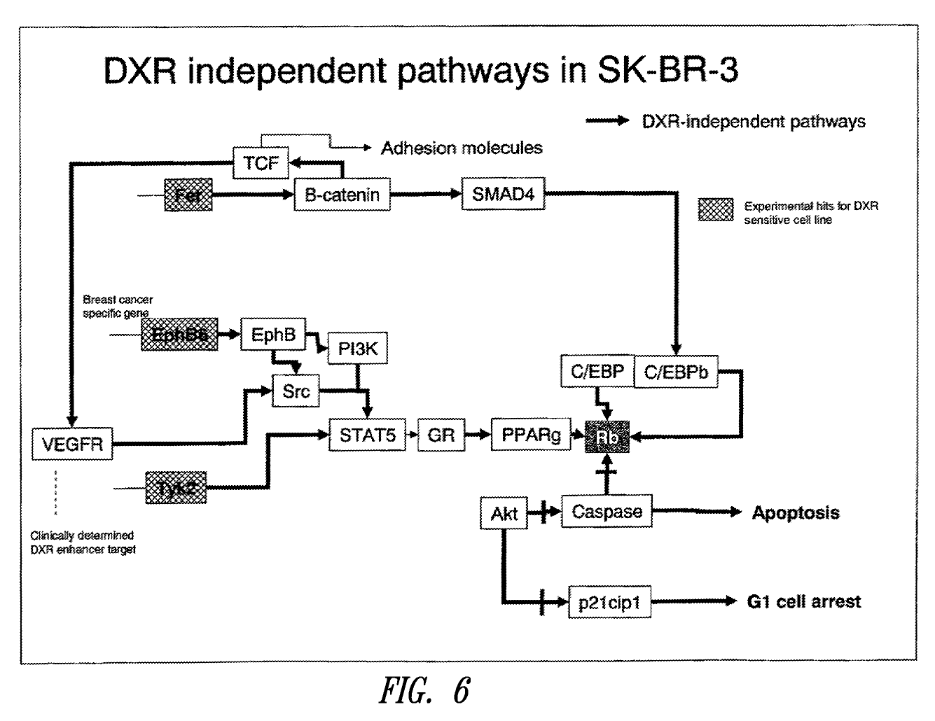

The above-mentioned pathway analysis as shown in Example 1 was also effective in analysis of pathway of growth of human derived breast cancer cells. The methods used herein are shown in FIGS. 2 and 5A-B. It was known that breast cancer cell SK-BR-3 is subjected to growth inhibition by anti-cancer doxorubicin (DXR). The component collection (8) which increases DXR sensitivity of SK-BR-3 were specified using RNA interference, and the collection {EPHA3, EPHA4, EPHB4, DDR1, EPHB6, FER, TYK2} were obtained. Next, in the absence of DXR, the component collection (9) which inhibits growth of SK-BR-3 was specified using the RNA interference method, and the collection {BTK, BLK, ERBB2, CSF1, EPHB6, FER, TYK2} was obtained. As a reference cell, T-47D cell, which has the same DXR sensitivity as the SK-BR-3 and is a human derived breast cancer, was used for detecting a component collection (10) which increases DXR sensitivity of T-47D cells, and component collection (11) which inhibits growth of T-47D cells in the absence of DXR, and the intersection (12) of component collection (10) and component collection (11) was obtained. Furthermore, intersection (13) of collections (8) and (9) was obtained. Intersection (14) of two intersections (12) and (13) was {EPHB6, FER, TYK2}. It turned out that intersection (14) and Rb, which directly control the cellular growth, share common pathway component collection (15) {PI3K, SRC, STAT5, GR, PPARg} (FIG. 6). FIG. 6 depicts an exemplary of extraction of common pathway for DXR independent pathways in SK-BR-3. The common molecules FER, EPHB6 and TYK2 experimentally elucidated are connected to the defined endpoint RB through the molecular relations described in the graph. Shaded boxes indicate experimental hits for DXR sensitive cell line.

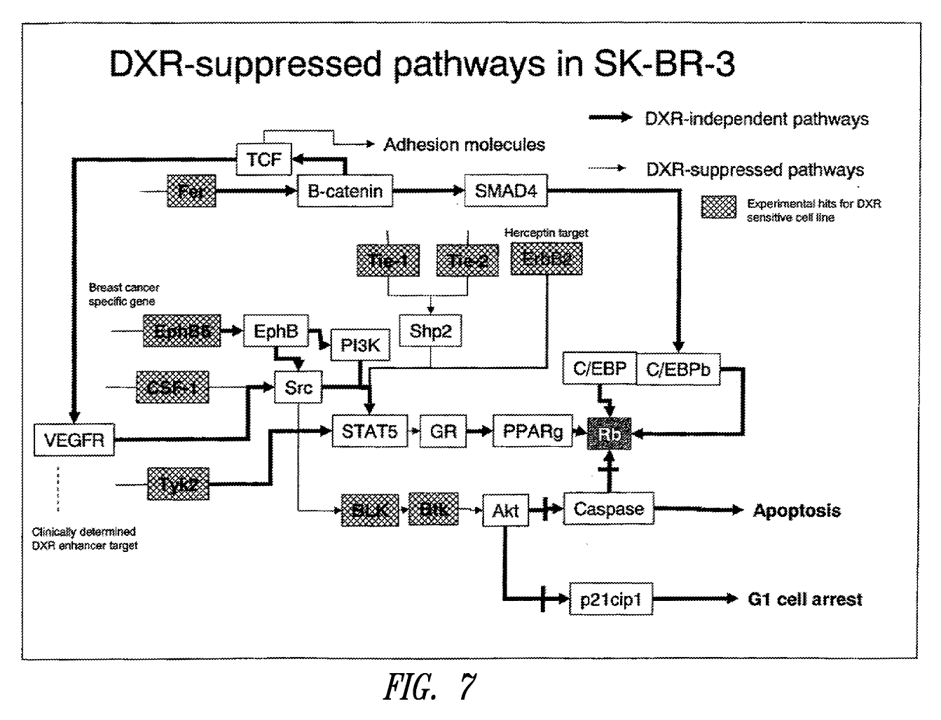

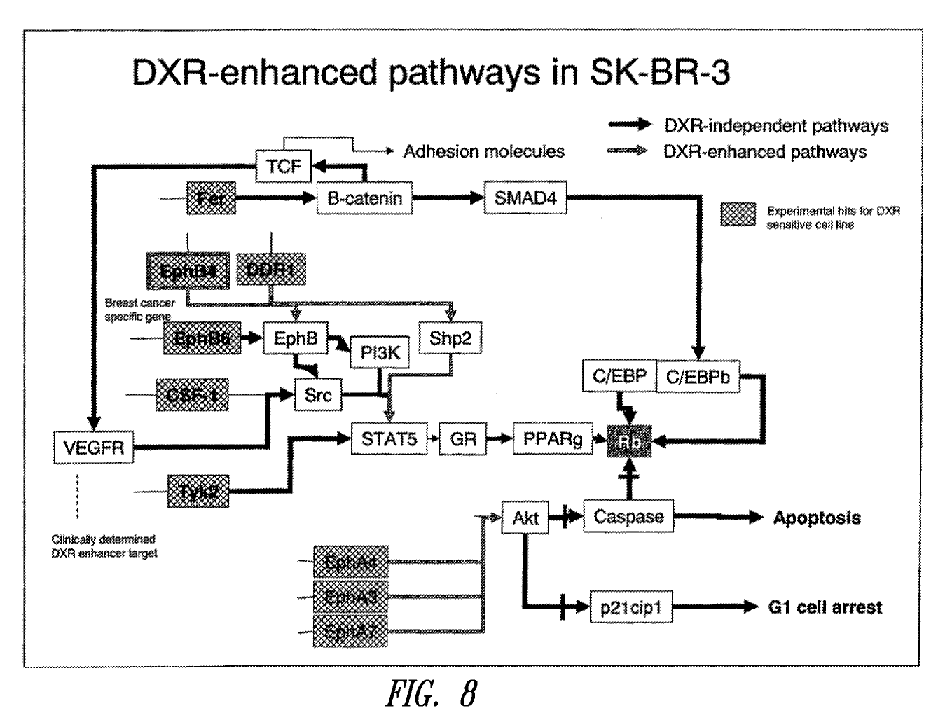

Furthermore, it also turned out that the component collections (8) and (9) which are subjected to the effects of DXR, are located upstream of the pathway (FIGS. 7 and 8). FIG. 7 depicts an exemplary of extraction of pathway which is inhibited by DXR, i.e. DXR-suppressed pathways in SK-BR-3. The molecules Tie-1, Tie-2, ERBB2, CSF-1, BLK, and BTK elucidated as the DXR-suppressed pathway components are connected to the defined endpoint RB through the molecular relations described in the graph. Shaded boxes indicate experimental hits for DXR sensitive cell line. FIG. 8 depicts an exemplary of extraction of pathway which is increased by DXR, i.e. DXR-enhanced pathways in SK-BR-3. The molecules EPHB4, DDR1, EPHA3, EPHA4, and EPHA7 elucidated as the DXR-enhanced pathway components are connected to the defined endpoint RB through the molecular relations described in the graph. Shaded boxes indicate experimental hits for DXR sensitive cell line.

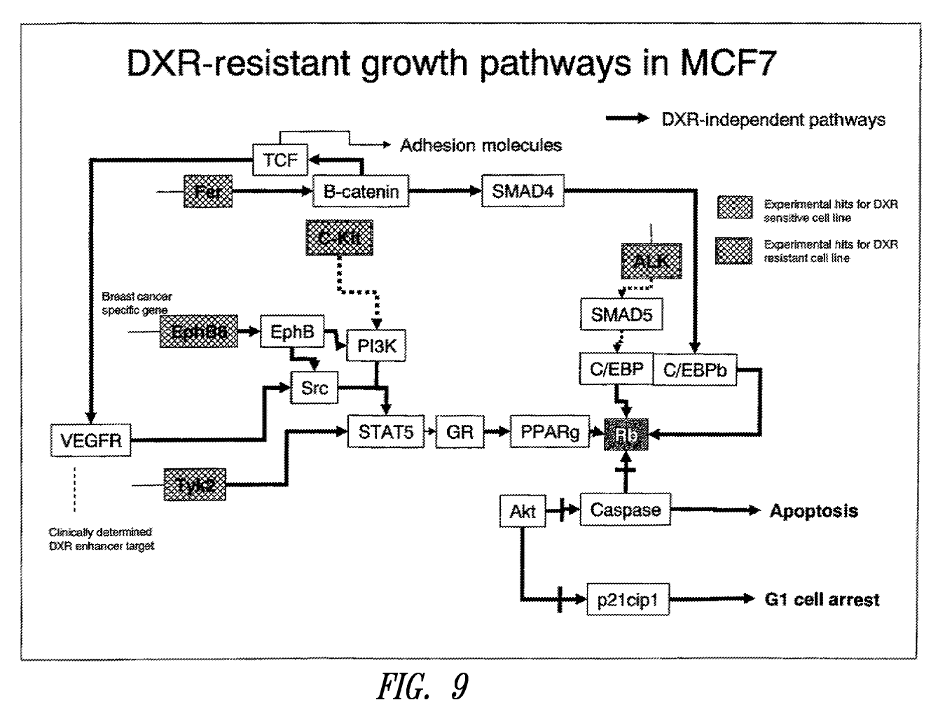

Moreover, component collection (16) which contributes to the growth of breast cancer cells MCF7 having DXR resistance was extracted by means of RNA interference experiments and thereby obtained {KIT, ALK}. This collection also turned out to be located upstream of collection (15) (FIG. 9). FIG. 9 depicts an exemplary extraction of growth of a cell having DXR resistance, i.e. DXR-resistant growth pathways in MCF7. The molecules C-KIT, and ALK elucidated as the DXR-resistant pathway components are connected to the defined endpoint RB through the molecular relations described in the graph.

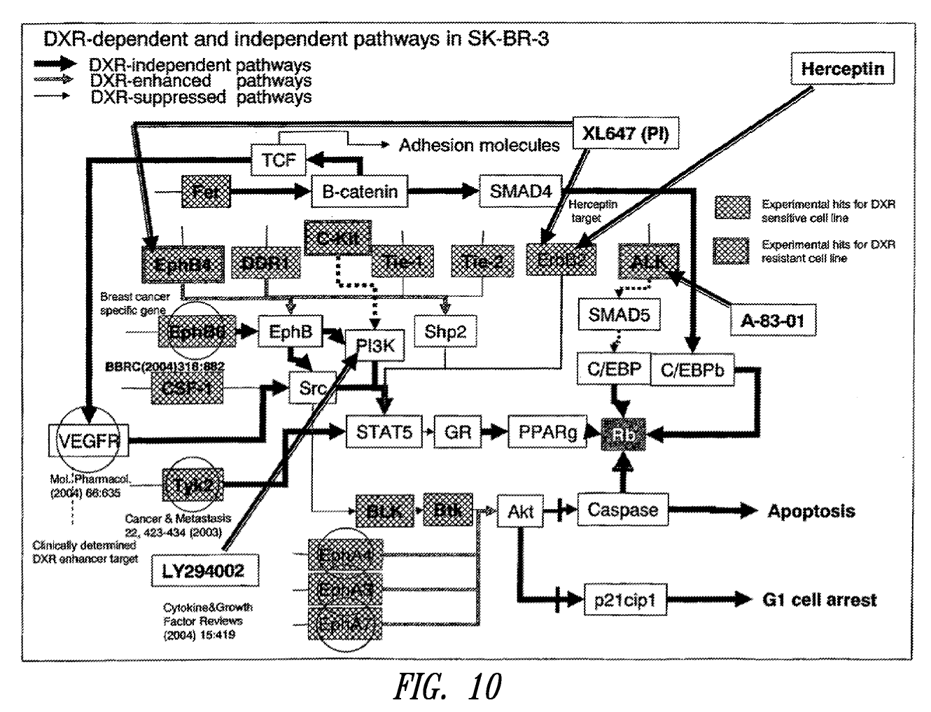

The overall DXR-dependent and independent pathways in SK-BR-3 is shown in FIG. 10. As shown in FIG. 10, the present invention elucidated how known anti-cancer agents function in the cells. Therefore, Herceptin and XL647(PI) affects on ErbB2 resulting in DXR-suppression. EphB6, VEFGR, Tyk2, EphA3, EphA4, and EphA7 turned out to be potential targets for screening anti-cancer agents (BBRC (2004) 318:882, Mol. Pharmacol. (2004) 66:635, Cancer & Metastasis 22, 423-434 (2003), Cytokine & Growth Factor Reviews (2004) 15:419). Recently, VEGFR has been clinically determined to be a DXR enhance target. Therefore, the present invention clearly demonstrates that it provides effective screening methods.

Although certain preferred embodiments have been described herein, it is not intended that such embodiments be construed as limitations on the scope of the invention except as set forth in the appended claims. Various other modifications and equivalents will be apparent to and can be readily made by those skilled in the art, after reading the description herein, without departing from the scope and spirit of this invention. All patents, published patent applications and publications cited herein are incorporated by reference as if set forth fully herein.

### INDUSTRIAL APPLICABILITY

According to the present invention, it is possible to effectively determine an upstream or downstream component of a component necessary for phenotypic alteration of a living organism by observing a surprisingly small number of factors. Therefore, the present invention is applicable to diagnosis, prevention, and treatment. The present invention is also applicable to the fields of food, cosmetics, agriculture, environmental engineering, and the like.

1. A method for deriving an upstream or downstream component of a component necessary for phenotypic alteration of a living organism, the method comprising the steps of:
A) specifying a pathway of interest related to the phenotypic alteration and a reference pathway different from the pathway of interest, and specifying a stimulant of interest and a reference stimulant which respectively stimulate the pathway of interest and the reference pathway;
B) giving the stimulant of interest to the living organism to identify a collection of components of interest necessary for the phenotypic alteration;
C) giving the reference stimulant to the living organism to identify a collection of reference components necessary for the phenotypic alteration;
D) calculating an intersection between the collection of the components of interest and the reference components; and
E) calculating differential collection by subtracting the intersection from the collection of components of interest, wherein a component which belongs to the differential collection is determined to be present upstream or downstream of the intersection.

2. The method according to claim 1, wherein the living organism is a cell.

3. The method according to claim 1, wherein the living organism is grown under two or more different conditions.

4. The method according to claim 1, wherein the component is induced from functional assay data which is indicative of a cell, tissue or an individual.

5. The method according to claim 1, wherein the component is selected based on a functional assay from a limited number of candidate genes including miRNA.

6. The method according to claim 1, wherein the component is a target calculated based on functional assay data from the target collection of the stimulant.

7. The method according to claim 1, wherein the component is a protein, a nucleic acid or both which has an affect on a phenotype of interest.

8. The method according to claim 1, wherein the component is derived from the result of a functional screening from a limited number of functional nucleic acid libraries.

9. The method according to claim 1, wherein the stimulant is an antibody, an RNA interference agent or a molecular target inhibitor.

10. A method for profiling a compound comprising the step of repeatedly applying the method according to claim 1.

11. A process for profiling a compound which can be combined for use in achieving the phenotypic alteration, the process comprising the step of repeatedly applying the method according to claim 1, wherein the process further comprises the steps of:
A) calculating the collection of components of interest which increases the phenotypic alteration expected by a living organism under a culture condition in which the compound is added;
B) calculating the collection of components of interest which increases the phenotypic alteration expected by a living organism under a culture condition in which there is no compound;
C) calculating a differential collection of the collection of components of interest and the collection of reference components thereby calculating a specific component collection appearing under the culture conditions with a compound added thereto;
D) calculating a common pathway of components included in the specific component collection; and
E) selecting a compound which targets the common pathway.

12. A process for searching for a target of a compound, comprising the method according to claim 1, the process further comprising the steps of:
A) calculating the collection of components of interest which increases the phenotypic alteration expected by a living organism under a culture condition in which the compound which can be combined for use in achieving the phenotypic alteration is added;
B) calculating the collection of components of interest which increases the phenotypic alteration expected by a living organism under a culture condition in which there is no compound which can be combined for use in achieving the phenotypic alteration;
C) calculating a differential collection of the collection of components of interest and the collection of reference components, thereby calculating a specific component collection appearing under the culture conditions with a compound added thereto;
D) calculating a common pathway of components included in the specific component collection; and
E) selecting a target included in the common pathway.

13. A process for searching for a target of a similar compound, comprising the method according to claim 1, wherein the process comprises the steps of:
A) calculating the collection of components of interest which increases the phenotypic alteration expected by a living organism in a culture circumstance in which the compound which can be combined for use in achieving the phenotypic alteration is added;
B) calculating the collection of components of interest which increases the phenotypic alteration expected by a living organism under a culture condition in which there is no compound which can be combined for use in achieving the phenotypic alteration;
C) calculating a differential collection of the collection of components of interest and the collection of reference components, thereby calculating a specific component collection appearing under the culture condition with a compound added thereto;
D) calculating a common pathway of components included in the specific component collection; and
E) selecting a target of a similar compound from the common pathway.

14. A method for inhibiting breast cancer using a combination of DXR and at least an inhibitor of the EphA family.

15. A method for inhibiting breast cancer using a combination of DXR and at least an inhibitor of the EphB family.

16. A method for inhibiting breast cancer using a combination of DXR and at least an inhibitor of c-KIT.

17. A method for inhibiting breast cancer using a combination of DXR and at least an inhibitor of ALK.

18. A system for deriving an upstream or downstream component necessary for the phenotypic alteration of a living organism, the system comprising:
A) a computer for specifying a pathway of interest related to the phenotypic alteration and a reference pathway different from the pathway of interest, and specifying a stimulant of interest and a reference stimulant which respectively stimulate the pathway of interest and the reference pathway;
B) an assay system for giving the stimulant of interest to the living organism to identify a collection of components of interest necessary for the phenotypic alteration;
C) an assay system for giving the reference stimulant to the living organism to identify a collection of reference components necessary for the phenotypic alteration;
D) a computer for calculating an intersection between the collection of the components of interest and the reference components; and
E) a computer for calculating differential collection by subtracting the intersection from the collection of components of interest, wherein a component which belongs to the differential collection is determined to be present upstream or downstream of the intersection.

19. A program for implementation by a computer to conduct a method for deriving an upstream or downstream component necessary for the phenotypic alteration of a living organism, the method comprising the steps of:
A) specifying a pathway of interest related to the phenotypic alteration and a reference pathway different from the pathway of interest, and specifying a stimulant of interest and a reference stimulant which respectively stimulate the pathway of interest and the reference pathway;
B) giving the stimulant of interest to the living organism to identify a collection of components of interest necessary for the phenotypic alteration;
C) giving the reference stimulant to the living organism to identify a collection of reference components necessary for the phenotypic alteration;
D) calculating an intersection between the collection of the components of interest and the reference components; and
E) calculating a differential collection by subtracting the intersection from the collection of components of interest, wherein a component which belongs to the differential collection is determined to be present upstream or downstream of the intersection.

20. A storage medium with a program stored thereon for an implementation by a computer to conduct a method for deriving upstream or downstream of a component necessary for phenotypic alteration of a living organism, the method comprising the steps of:
A) specifying a pathway of interest related to the phenotypic alteration and a reference pathway different from the pathway of interest, and specifying a stimulant of interest and a reference stimulant which respectively stimulates the pathway of interest and the reference pathway;
B) giving the stimulant of interest to the living organism to identify a collection of components of interest necessary for the phenotypic alteration;
C) giving the reference stimulant to the living organism to identify a collection of reference components necessary for the phenotypic alteration;
D) calculating an intersection between the collection of the components of interest and the reference components; and
E) calculating a differential collection by subtracting the intersection from the collection of components of interest, wherein a component which belongs to the differential collection is determined to be present upstream or downstream of the intersection.

21. A composition for inhibiting breast cancer comprising a combination of DXR and at least an inhibitor of the EphA family.

22. A composition for inhibiting breast cancer comprising a combination of DXR and at least an inhibitor of the EphB family.

23. A composition for inhibiting breast cancer comprising a combination of DXR and at least an inhibitor of c-KIT.

24. A composition for inhibiting breast cancer comprising a combination of DXR and at least an inhibitor of ALK.


SEQUENCE LISTING (26 sequences)

SEQ ID NO: 1 - DNA, 3367 bp, Homo sapiens
  Inventor MIYAKE, Masato; FUJITA, Yoshiji
gcccccgcccggcccgccccgctctcctagtcccttgcaacctggcgctgccatccgggccactgtcccaggtcccggcccggagctatggagcggcgctggcccctggggctagggctggtgctgctgctctgcgccccgctgcccccgggggcgcgcgccaaggaagttactctgatggacacaagcaaggcacagggagagctgggctggctgctggatcccccaaaagatgggtggagtgaacagcaacagatactgaatgggacacccctgtacatgtaccaggactgcccaatgcaaggacgcagagacactgaccactggcttcgctccaattggatctaccgcggggaggaggcttcccgcgtccacgtggagctgcagttcaccgtgcgggactgcaagagtttccctgggggagccgggcctctgggctgcaaggagaccttcaaccttctgtacatggagagtgaccaggatgtgggcattcagctccgacggcccttgttccagaaggtaaccacggtggctgcagaccagagcttcaccattcgagaccttgcgtctggctccgtgaagctgaatgtggagcgctgctctctgggccgcctgacccgccgtggcctctacctcgctttccacaacccgggtgcctgtgtggccctggtgtctgtccgggtcttctaccagcgctgtcctgagaccctgaatggcttggcccaattcccagacactctgcctggccccgctgggttggtggaagtggcggggacctgcttgccccacgcgcgggccagccccaggccctcaggtgcaccccgcatgcactgcagccctgatggcgagtggctggtgcctgtaggacggtgccactgtgagcctggctatgaggaaggtggcagtggcgaagcatgtgttgcctgccctagcggctcctaccggatggacatggacacaccccattgtctcacgtgcccccagcagagcactgctgagtctgagggggccaccatctgtacctgtgagagcggccattacagagctcccggggagggcccccaggtggcatgcacaggtcccccctcggccccccgaaacctgagcttctctgcctcagggactcagctctccctgcgttgggaacccccagcagatacggggggacgccaggatgtcagatacagtgtgaggtgttcccagtgtcagggcacagcacaggacggggggccctgccagccctgtggggtgggcgtgcacttctcgccgggggcccgggggctcaccacacctgcagtgcatgtcaatggccttgaaccttatgccaactacacctttaatgtggaagcccaaaatggagtgtcagggctgggcagctctggccatgccagcacctcagtcagcatcagcatggggcatgcagagtcactgtcaggcctgtctctgagactggtgaagaaagaaccgaggcaactagagctgacctgggcggggtcccggccccgaagccctggggcgaacctgacctatgagctgcacgtgctgaaccaggatgaagaacggtaccagatggttctagaacccagggtcttgctgacagagctgcagcctgacaccacatacatcgtcagagtccgaatgctgaccccactgggtcctggccctttctcccctgatcatgagtttcggaccagcccaccagtgtccaggggcctgactggaggagagattgtagccgtcatctttgggctgctgcttggtgcagccttgctgcttgggattctcgttttccggtccaggagagcccagcggcagaggcagcagaggcagcgtgaccgcgccaccgatgtggatcgagaggacaagctgtggctgaagccttatgtggacctccaggcatacgaggaccctgcacagggagccctggactttacccgggagcttgatccagcgtggctgatggtggacactgtcataggagaaggagagtttggggaagtgtatcgagggaccctgaggctccccagccaggactgcaagactgtggccattaagaccttaaaagacacatccccaggtggccagtggtggaacttccttcgagaggcaactatcatgggccagtttagccacccgcatattctgcatctggaaggcgtcgtcacaaagcgaaagccgatcatgatcatcacagaatttatggagaatggagccctggatgccttcctgagggagcgggaggaccagctggtccctgggcagctagtggccatgctgcagggcatagcatctggcatgaactacctcagtaatcacaattatgtccaccgggacctggctgccagaaacatcttggtgaatcaaaacctgtgctgcaaggtgtctgactttggcctgactcgcctcctggatgactttgatggcacatacgaaacccagggaggaaagatccctatccgttggacagcccctgaagccattgcccatcggatcttcaccacagccagcgatgtgtggagctttgggattgtgatgtgggaggtgctgagctttggggacaagccttatggggagatgagcaatcaggaggttatgaagagcattgaggatgggtaccggttgccccctcctgtggactgccctgcccctctgtatgagctcatgaagaactgctgggcatatgaccgtgcccgccggccacacttccagaagcttcaggcacatctggagcaactgcttgccaacccccactccctgcggaccattgccaactttgaccccaggatgactcttcgcctgcccagcctgagtggctcagatgggatcccatatcgaaccgtctctgagtggctcgagtccatacgcatgaaacgctacatcctgcacttccactcggctgggctggacaccatggagtgtgtgctggagctgaccgctgaggacctgacgcagatgggaatcacactgcccgggcaccagaagcgcattctttgcagtattcagggattcaaggactgatccctcctctcaccccatgcccaatcagggtgcaaggagcaaggacggggccaaggtcgctcatggtcactccctgcgccccttcccacaacctgccagactaggctatcggtgctgcttctgcccactttcaggagaaccctgctctgcaccccagaaaacctctttgttttaaaagggaggtgggggtagaagtaaaaggatgatcatgggagggagctgaggggttaatatatatacatacatacacatatatatatttttgtaaataaacaggaactgattttctgcctccatcccacccatgagggctgcaggcactacaaaagagctgactactgagaaaaaa

SEQ ID NO: 2 - protein, 976 aa, Homo sapiens
MERRWPLGLGLVLLLCAPLPPGARAKEVTLMDTSKAQGELGWLLDPPKDGWSEQQQILNGTPLYMYQDCPMQGRRDTDHWLRSNWIYRGEEASRVHVELQFTVRDCKSFPGGAGPLGCKETFNLLYMESDQDVGIQLRRPLFQKVTTVAADQSFTIRDLASGSVKLNVERCSLGRLTRRGLYLAFHNPGACVALVSVRVFYQRCPETLNGLAQFPDTLPGPAGLVEVAGTCLPHARASPRPSGAPRMHCSPDGEWLVPVGRCHCEPGYEEGGSGEACVACPSGSYRMDMDTPHCLTCPQQSTAESEGATICTCESGHYRAPGEGPQVACTGPPSAPRNLSFSASGTQLSLRWEPPADTGGRQDVRYSVRCSQCQGTAQDGGPCQPCGVGVHFSPGARGLTTPAVHVNGLEPYANYTFNVEAQNGVSGLGSSGHASTSVSISMGHAESLSGLSLRLVKKEPRQLELTWAGSRPRSPGANLTYELHVLNQDEERYQMVLEPRVLLTELQPDTTYIVRVRMLTPLGPGPFSPDHEFRTSPPVSRGLTGGEIVAVIFGLLLGAALLLGILVFRSRRAQRQRQQRQRDRATDVDREDKLWLKPYVDLQAYEDPAQGALDFTRELDPAWLMVDTVIGEGEFGEVYRGTLRLPSQDCKTVAIKTLKDTSPGGQWWNFLREATIMGQFSHPHILHLEGVVTKRKPIMIITEFMENGALDAFLREREDQLVPGQLVAMLQGIASGMNYLSNHNYVHRDLAARNILVNQNLCCKVSDFGLTRLLDDFDGTYETQGGKIPIRWTAPEAIAHRIFTTASDVWSFGIVMWEVLSFGDKPYGEMSNQEVMKSIEDGYRLPPPVDCPAPLYELMKNCWAYDRARRPHFQKLQAHLEQLLANPHSLRTIANFDPRMTLRLPSLSGSDGIPYRTVSEWLESIRMKRYILHFHSAGLDTMECVLELTAEDLTQMGITLPGHQKRILCSIQGFKD

SEQ ID NO: 3 - DNA, 3963 bp, Homo sapiens
  Inventor MIYAKE, Masato; FUJITA, Yoshiji
attaaggactcggggcaggaggggcagaagttgcgcgcaggccggcgggcgggagcggacaccgaggccggcgtgcaggcgtgcgggtgtgcgggagccgggctcggggggatcggaccgagagcgagaagcgcggcatggagctccaggcagcccgcgcctgcttcgccctgctgtggggctgtgcgctggccgcggccgcggcggcgcagggcaaggaagtggtactgctggactttgctgcagctggaggggagctcggctggctcacacacccgtatggcaaagggtgggacctgatgcagaacatcatgaatgacatgccgatctacatgtactccgtgtgcaacgtgatgtctggcgaccaggacaactggctccgcaccaactgggtgtaccgaggagaggctgagcgtatcttcattgagctcaagtttactgtacgtgactgcaacagcttccctggtggcgccagctcctgcaaggagactttcaacctctactatgccgagtcggacctggactacggcaccaacttccagaagcgcctgttcaccaagattgacaccattgcgcccgatgagatcaccgtcagcagcgacttcgaggcacgccacgtgaagctgaacgtggaggagcgctccgtggggccgctcacccgcaaaggcttctacctggccttccaggatatcggtgcctgtgtggcgctgctctccgtccgtgtctactacaagaagtgccccgagctgctgcagggcctggcccacttccctgagaccatcgccggctctgatgcaccttccctggccactgtggccggcacctgtgtggaccatgccgtggtgccaccggggggtgaagagccccgtatgcactgtgcagtggatggcgagtggctggtgcccattgggcagtgcctgtgccaggcaggctacgagaaggtggaggatgcctgccaggcctgctcgcctggattttttaagtttgaggcatctgagagcccctgcttggagtgccctgagcacacgctgccatcccctgagggtgccacctcctgcgagtgtgaggaaggcttcttccgggcacctcaggacccagcgtcgatgccttgcacacgacccccctccgccccacactacctcacagccgtgggcatgggtgccaaggtggagctgcgctggacgccccctcaggacagcgggggccgcgaggacattgtctacagcgtcacctgcgaacagtgctggcccgagtctggggaatgcgggccgtgtgaggccagtgtgcgctactcggagcctcctcacggactgacccgcaccagtgtgacagtgagcgacctggagccccacatgaactacaccttcaccgtggaggcccgcaatggcgtctcaggcctggtaaccagccgcagcttccgtactgccagtgtcagcatcaaccagacagagccccccaaggtgaggctggagggccgcagcaccacctcgcttagcgtctcctggagcatccccccgccgcagcagagccgagtgtggaagtacgaggtcacttaccgcaagaagggagactccaacagctacaatgtgcgccgcaccgagggtttctccgtgaccctggacgacctggccccagacaccacctacctggtccaggtgcaggcactgacgcaggagggccagggggccggcagcaaggtgcacgaattccagacgctgtccccggagggatctggcaacttggcggtgattggcggcgtggctgtcggtgtggtcctgcttctggtgctggcaggagttggcttctttatccaccgcaggaggaagaaccagcgtgcccgccagtccccggaggacgtttacttctccaagtcagaacaactgaagcccctgaagacatacgtggacccccacacatatgaggaccccaaccaggctgtgttgaagttcactaccgagatccatccatcctgtgtcactcggcagaaggtgatcggagcaggagagtttggggaggtgtacaagggcatgctgaagacatcctcggggaagaaggaggtgccggtggccatcaagacgctgaaagccggctacacagagaagcagcgagtggacttcctcggcgaggccggcatcatgggccagttcagccaccacaacatcatccgcctagagggcgtcatctccaaatacaagcccatgatgatcatcactgagtacatggagaatggggccctggacaagttccttcgggagaaggatggcgagttcagcgtgctgcagctggtgggcatgctgcggggcatcgcagctggcatgaagtacctggccaacatgaactatgtgcaccgtgacctggctgcccgcaacatcctcgtcaacagcaacctggtctgcaaggtgtctgactttggcctgtcccgcgtgctggaggacgaccccgaggccacctacaccaccagtggcggcaagatccccatccgctggaccgccccggaggccatttcctaccggaagttcacctctgccagcgacgtgtggagctttggcattgtcatgtgggaggtgatgacctatggcgagcggccctactgggagttgtccaaccacgaggtgatgaaagccatcaatgatggcttccggctccccacacccatggactgcccctccgccatctaccagctcatgatgcagtgctggcagcaggagcgtgcccgccgccccaagttcgctgacatcgtcagcatcctggacaagctcattcgtgcccctgactccctcaagaccctggctgactttgacccccgcgtgtctatccggctccccagcacgagcggctcggagggggtgcccttccgcacggtgtccgagtggctggagtccatcaagatgcagcagtatacggagcacttcatggcggccggctacactgccatcgagaaggtggtgcagatgaccaacgacgacatcaagaggattggggtgcggctgcccggccaccagaagcgcatcgcctacagcctgctgggactcaaggaccaggtgaacactgtggggatccccatctgagcctcgacagggcctggagccccatcggccaagaatacttgaagaaacagagtggcctccctgctgtgccatgctgggccactggggactttatttatttctagttctttcctccccctgcaacttccgctgaggggtctcggatgacaccctggcctgaactgaggagatgaccagggatgctgggctgggccctctttccctgcgagacgcacacagctgagcacttagcaggcaccgccacgtcccagcatccctggagcaggagccccgccacagccttcggacagacatataggatattcccaagccgaccttccctccgccttctcccacatgaggccatctcaggagatggagggcttggcccagcgccaagtaaacagggtacctcaagccccatttcctcacactaagagggcagactgtgaacttgactgggtgagacccaaagcggtccctgtccctctagtgccttctttagaccctcgggccccatcctcatccctgactggccaaacccttgctttcctgggcctttgcaagatgcttggttgtgttgaggtttttaaatatatattttgtactttgtggagagaatgtgtgtgtgtggcagggggccccgccagggctggggacagagggtgtcaaacattcgtgagctggggactcagggaccggtgctgcaggagtgtcctgcccatgccccagtcggccccatctctcatccttttggataagtttctattctgtcagtgttaaagattttgttttgttggacatttttttcgaatcttaatttattattttttttatatttattgttagaaaatgacttatttctgctctggaataaagttgcagatgattcaaaccgaaaaaaaaaaaaaaaaaa

SEQ ID NO: 4 - protein, 976 aa, Homo sapiens
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 5 - DNA, 5826 bp, Homo sapiens
  Inventor MIYAKE, Masato; FUJITA, Yoshiji
cccgctctgcttcagcgcacgctgaagacggcactaggacccagggaagtccccgagcggggttcgcggaaaggcagccagactcctccttatctccagtgtcaaacttgacatcagcctgcgagcggagcatggtaacttctccagcaatcagagcgctccccctcacatcagtggcatgcttcatggagatatgctcctctcactgccctctgcaccagcaacatggattgtcagctctccatcctcctccttctcagctgctctgttctcgacagcttcggggaactgattccgcagccttccaatgaagtcaatctactggattcaaaaacaattcaaggggagctgggctggatctcttatccatcacatgggtgggaagagatcagtggtgtggatgaacattacacacccatcaggacttaccaggtgtgcaatgtcatggaccacagtcaaaacaattggctgagaacaaactgggtccccaggaactcagctcagaagatttatgtggagctcaagttcactctacgagactgcaatagcattccattggttttaggaacttgcaaggagacattcaacctgtactacatggagtctgatgatgatcatggggtgaaatttcgagagcatcagtttacaaagattgacaccattgcagctgatgaaagtttcactcaaatggatcttggggaccgtattctgaagctcaacactgagattagagaagtaggtcctgtcaacaagaagggattttatttggcatttcaagatgttggtgcttgtgttgccttggtgtctgtgagagtatacttcaaaaagtgcccatttacagtgaagaatctggctatgtttccagacacggtacccatggactcccagtccctggtggaggttagagggtcttgtgtcaacaattctaaggaggaagatcctccaaggatgtactgcagtacagaaggcgaatggcttgtacccattggcaagtgttcctgcaatgctggctatgaagaaagaggttttatgtgccaagcttgtcgaccaggtttctacaaggcattggatggtaatatgaagtgtgctaagtgcccgcctcacagttctactcaggaagatggttcaatgaactgcaggtgtgagaataattacttccgggcagacaaagaccctccatccatggcttgtacccgacctccatcttcaccaagaaatgttatctctaatataaacgagacctcagttatcctggactggagttggcccctggacacaggaggccggaaagatgttaccttcaacatcatatgtaaaaaatgtgggtggaatataaaacagtgtgagccatgcagcccaaatgtccgcttcctccctcgacagtttggactcaccaacaccacggtgacagtgacagaccttctggcacatactaactacacctttgagattgatgccgttaatggggtgtcagagctgagctccccaccaagacagtttgctgcggtcagcatcacaactaatcaggctgctccatcacctgtcctgacgattaagaaagatcggacctccagaaatagcatctctttgtcctggcaagaacctgaacatcctaatgggatcatattggactacgaggtcaaatactatgaaaagcaggaacaagaaacaagttataccattctgagggcaagaggcacaaatgttaccatcagtagcctcaagcctgacactatatacgtattccaaatccgagcccgaacagccgctggatatgggacgaacagccgcaagtttgagtttgaaactagtccagactctttctccatctctggtgaaagtagccaagtggtcatgatcgccatttcagcggcagtagcaattattctcctcactgttgtcatctatgttttgattgggaggttctgtggctataagtcaaaacatggggcagatgaaaaaagacttcattttggcaatgggcatttaaaacttccaggtctcaggacttatgttgacccacatacatatgaagaccctacccaagctgttcatgagtttgccaaggaattggatgccaccaacatatccattgataaagttgttggagcaggtgaatttggagaggtgtgcagtggtcgcttaaaacttccttcaaaaaaagagatttcagtggccattaagaccctgaaagttggctacacagaaaagcagaggagagacttcctgggagaagcaagcattatgggacagtttgaccaccccaatatcattcgactggaaggagttgttaccaaaagtaagccagttatgattgtcacagaatacatggagaatggttccttggatagtttcctacgtaaacacgatgcccagtttactgtcattcagctagtggggatgcttcgagggatagcatctggcatgaagtacctgtcagacatgggctatgttcaccgagacctcgctgctcggaacatcttgatcaacagtaacttggtgtgtaaggtttctgatttcggactttcgcgtgtcctggaggatgacccagaagctgcttatacaacaagaggagggaagatcccaatcaggtggacatcaccagaagctatagcctaccgcaagttcacgtcagccagcgatgtatggagttatgggattgttctctgggaggtgatgtcttatggagagagaccatactgggagatgtccaatcaggatgtaattaaagctgtagatgagggctatcgactgccaccccccatggactgcccagctgccttgtatcagctgatgctggactgctggcagaaagacaggaacaacagacccaagtttgagcagattgttagtattctggacaagcttatccggaatcccggcagcctgaagatcatcaccagtgcagccgcaaggccatcaaaccttcttctggaccaaagcaatgtggatatcactaccttccgcacaacaggtgactggcttaatggtgtctggacagcacactgcaaggaaatcttcacgggtgtggagtacagttcttgtgacacaatagccaagatttccacagatgacatgaaaaaggttggtgtcaccgtggttgggccacagaagaagatcatcagtagcattaaagctctagaaacgcaatcaaagaatggcccagttcccgtgtaaagcacgggacggaagtgcttctggacggaagtggtggctgtggaaggcgtagcatcatcctgcagacagacaataattctggagatactggtggaagttccaagtccaataagacactcaaatatgagtacaaatgccttaaaatggaattgaaaaactctttattttcccctatcatttattggatgggtgggtggggtatttttttgtaattgcttttttaaatattagttaatggattaaatttaattcttcagcgtaaaatggtgaagaactagcatatagccattgatcataaactgactatcataaaatcaaaacaagtgaaataacaaaatggacatggtggctttgtttaggtagagccacaaaagaaaagacttgtaatatttttatatacagaggaaatctgtaacaggtattttgtttcttttaaagcaagcaacacagaggaatttatacctcaaactatctggccatatttactaccttatcactgcattattctcttttatctgtttaaagcatatagagatgaagtttgtagttgttttaagtactacacatttttaaattgttagcttccttaagtatatcatgtaaagaaatgtcttaatttttgaaaaaagtacatatttattttcttttgaattgtttttattgttttctatttatgccttgatgatttaatatggatttgttacagccaagtgccaaatgctctctcaaattgtcagcaatttaactagacacagataataatgggtttctttcagattttttgaaccatccacttacatatatttttaaaaaatgaaatccttttcctgttcatacactaaccaaatctctcaaatctgttatcccaatcattgttgcctctccgtttattataaactgtatgctcacaacttagtgtaatataccagcttgtatgcaatggattttcaaccagataacatacctttcctgctctggtgcttagagactatcaactccctcctttagtgaaggagccgtgttagagcttccgagaatagctccactggagagaagtggaatcctatatagaatgctgcactaattgacaacacagcctataggccaatgcatgagtaaaaaaaaaaacaattactggctcactggctttgaaaagtcacttactattgttgctgaaacttgctgagctgtttatagagaatgatgataacagaacttttcctctgtatcactggtgtttaggtgaattaattaaacattgtgatcattagtaccaggtattattatctttaagagtcttccacttcaatgcacatggtgcagttttggtgtgtaacttagaaggattgaacttctttgaatttactggacataacattttcagaatagttggtcatctagcaaccgcctcaaaatgtgtaagcaggagagaaatttctcatcacagggatttagacttactattacataaaggctaactatgagcttgctcattaattttgaaaagatgtacctggtggatatctagctagtaatatattctgaagcaacattttagctctattgatactctttctaatgctgatatgatcttgagtataagaaatgcatatgtcactagaatggataaaataatgctgcaaacttaatgttcttatgcaaaatggaacgctaatgaaacacagcttacaatcgcaaatcaaaactcacaagtgctcatctgttgtagatttagtgtaataagacttagattgtgctccttcggatatgattgtttctcaaatcttggcaatattccttagtcaaatcaggctactagaattctgtattggatatataagagcatgaaatttttaaaaatacacttgtgattataaaattaatcacaaatttcacttatacctgctatcagcagctagaaaacattttttttttaaatcaagtattttgtgtttggaatgttagaatgagatctgaatgtggtttcaatctaattttttcccagactactattttcttttttaggtactattctgagcatactcaacaaaacccatgcatttcataaactaatagaagttgaggattgttgaatctatttcacttattttggctgtggtttccatctgaaagtagaggttgtatacaccatatactgttcttcattttattaatatttttctccttgacctctcataaatttactttacacaattcttaccctgtacatatgtaaacataagtgtacgattcttaaccatggagtagaggtactagaatgcttacggccatctctttgtacaggaactgcattgactttcagtaaacataaagccacaactcctacatgatgttatgtaccatatgatctgttttgtatcttaaatttgatttacatatattatttatttctggtaactcactcagtttatgctgtgctaaatatcaatcaagccatgtataaatgtgatatgattggcaatatgtgtttactttaaacttgtcttttcaaaatattactcagtttatgttgtacaatgtagatggcctcttactaatgtaaaatgatttgtagtggaaacatttatatttttataataaacataatgaaaatattttttacagattggaaaaaaaaaaaaaaaaaaa

SEQ ID NO: 6 - protein, 983 aa, Homo sapiens
MDCQLSILLLLSCSVLDSFGELIPQPSNEVNLLDSKTIQGELGWISYPSHGWEEISGVDEHYTPIRTYQVCNVMDHSQNNWLRTNWVPRNSAQKIYVELKFTLRDCNSIPLVLGTCKETFNLYYMESDDDHGVKFREHQFTKIDTIAADESFTQMDLGDRILKLNTEIREVGPVNKKGFYLAFQDVGACVALVSVRVYFKKCPFTVKNLAMFPDTVPMDSQSLVEVRGSCVNNSKEEDPPRMYCSTEGEWLVPIGKCSCNAGYEERGFMCQACRPGFYKALDGNMKCAKCPPHSSTQEDGSMNCRCENNYFRADKDPPSMACTRPPSSPRNVISNINETSVILDWSWPLDTGGRKDVTFNIICKKCGWNIKQCEPCSPNVRFLPRQFGLTNTTVTVTDLLAHTNYTFEIDAVNGVSELSSPPRQFAAVSITTNQAAPSPVLTIKKDRTSRNSISLSWQEPEHPNGIILDYEVKYYEKQEQETSYTILRARGTNVTISSLKPDTIYVFQIRARTAAGYGTNSRKFEFETSPDSFSISGESSQVVMIAISAAVAIILLTVVIYVLIGRFCGYKSKHGADEKRLHFGNGHLKLPGLRTYVDPHTYEDPTQAVHEFAKELDATNISIDKVVGAGEFGEVCSGRLKLPSKKEISVAIKTLKVGYTEKQRRDFLGEASIMGQFDHPNIIRLEGVVTKSKPVMIVTEYMENGSLDSFLRKHDAQFTVIQLVGMLRGIASGMKYLSDMGYVHRDLAARNILINSNLVCKVSDFGLSRVLEDDPEAAYTTRGGKIPIRWTSPEAIAYRKFTSASDVWSYGIVLWEVMSYGERPYWEMSNQDVIKAVDEGYRLPPPMDCPAALYQLMLDCWQKDRNNRPKFEQIVSILDKLIRNPGSLKIITSAAARPSNLLLDQSNVDITTFRTTGDWLNGVWTAHCKEIFTGVEYSSCDTIAKISTDDMKKVGVTVVGPQKKIISSIKALETQSKNGPVPV

SEQ ID NO: 7 - DNA, 6364 bp, Homo sapiens
  Inventor MIYAKE, Masato; FUJITA, Yoshiji
ctgggatagaagcggcaggagcagcgttggcaccggcgaaccatggctgggattttctatttcgccctattttcgtgtctcttcgggatttgcgacgctgtcacaggttccagggtataccccgcgaatgaagttaccttattggattccagatctgttcagggagaacttgggtggatagcaagccctctggaaggagggtgggaggaagtgagtatcatggatgaaaaaaatacaccaatccgaacctaccaagtgtgcaatgtgatggaacccagccagaataactggctacgaactgattggatcacccgagaaggggctcagagggtgtatattgagattaaattcaccttgagggactgcaatagtcttccgggcgtcatggggacttgcaaggagacgtttaacctgtactactatgaatcagacaacgacaaagagcgtttcatcagagagaaccagtttgtcaaaattgacaccattgctgctgatgagagcttcacccaagtggacattggtgacagaatcatgaagctgaacaccgagatccgggatgtagggccattaagcaaaaaggggttttacctggcttttcaggatgtgggggcctgcatcgccctggtatcagtccgtgtgttctataaaaagtgtccactcacagtccgcaatctggcccagtttcctgacaccatcacaggggctgatacgtcttccctggtggaagttcgaggctcctgtgtcaacaactcagaagagaaagatgtgccaaaaatgtactgtggggcagatggtgaatggctggtacccattggcaactgcctatgcaacgctgggcatgaggagcggagcggagaatgccaagcttgcaaaattggatattacaaggctctctccacggatgccacctgtgccaagtgcccaccccacagctactctgtctgggaaggagccacctcgtgcacctgtgaccgaggctttttcagagctgacaacgatgctgcctctatgccctgcacccgtccaccatctgctcccctgaacttgatttcaaatgtcaacgagacatctgtgaacttggaatggagtagccctcagaatacaggtggccgccaggacatttcctataatgtggtatgcaagaaatgtggagctggtgaccccagcaagtgccgaccctgtggaagtggggtccactacaccccacagcagaatggcttgaagaccaccaaagtctccatcactgacctcctagctcataccaattacacctttgaaatctgggctgtgaatggagtgtccaaatataaccctaacccagaccaatcagtttctgtcactgtgaccaccaaccaagcagcaccatcatccattgctttggtccaggctaaagaagtcacaagatacagtgtggcactggcttggctggaaccagatcggcccaatggggtaatcctggaatatgaagtcaagtattatgagaaggatcagaatgagcgaagctatcgtatagttcggacagctgccaggaacacagatatcaaaggcctgaaccctctcacttcctatgttttccacgtgcgagccaggacagcagctggctatggagacttcagtgagcccttggaggttacaaccaacacagtgccttcccggatcattggagatggggctaactccacagtccttctggtctctgtctcgggcagtgtggtgctggtggtaattctcattgcagcttttgtcatcagccggagacggagtaaatacagtaaagccaaacaagaagcggatgaagagaaacatttgaatcaaggtgtaagaacatatgtggacccctttacgtacgaagatcccaaccaagcagtgcgagagtttgccaaagaaattgacgcatcctgcattaagattgaaaaagttataggagttggtgaatttggtgaggtatgcagtgggcgtctcaaagtgcctggcaagagagagatctgtgtggctatcaagactctgaaagctggttatacagacaaacagaggagagacttcctgagtgaggccagcatcatgggacagtttgaccatccgaacatcattcacttggaaggcgtggtcactaaatgtaaaccagtaatgatcataacagagtacatggagaatggctccttggatgcattcctcaggaaaaatgatggcagatttacagtcattcagctggtgggcatgcttcgtggcattgggtctgggatgaagtatttatctgatatgagctatgtgcatcgtgatctggccgcacggaacatcctggtgaacagcaacttggtctgcaaagtgtctgattttggcatgtcccgagtgcttgaggatgatccggaagcagcttacaccaccaggggtggcaagattcctatccggtggactgcgccagaagcaattgcctatcgtaaattcacatcagcaagtgatgtatggagctatggaatcgttatgtgggaagtgatgtcgtacggggagaggccctattgggatatgtccaatcaagatgtgattaaagccattgaggaaggctatcggttaccccctccaatggactgccccattgcgctccaccagctgatgctagactgctggcagaaggagaggagcgacaggcctaaatttgggcagattgtcaacatgttggacaaactcatccgcaaccccaacagcttgaagaggacagggacggagagctccagacctaacactgccttgttggatccaagctcccctgaattctctgctgtggtatcagtgggcgattggctccaggccattaaaatggaccggtataaggataacttcacagctgctggttataccacactagaggctgtggtgcacgtgaaccaggaggacctggcaagaattggtatcacagccatcacgcaccagaataagattttgagcagtgtccaggcaatgcgaacccaaatgcagcagatgcacggcagaatggttcccgtctgagccagtactgaataaactcaaaactcttgaaattagtttacctcatccatgcactttaattgaagaactgcactttttttacttcgtcttcgccctctgaaattaaagaaatgaaaaaaaaaaacaatatctgcagcgttgcttggtgcacagattgctgaaactgtggggcttacagaaatgactgccggtcatttgaatgagacctggaacaaatcgtttctcagaagtacttttctgttcatcaccagtctgtaaaatacatgtacctatagaaatagaacactgcctctgagttttgatgctgtatttgctgccagacactgagcttctgagacatccctgattctctctccatttggaattacaaccattgtattttgtttgtggcataaattacagtcatctgtctttcactggaatgaagaccatgcctaggaacattttttaaggactcagctgtggcttttagggcttggttcataccatgggggaaaaaaaagtcctaggagaaagcgacgtggctcattagtgttgcctcttcagtgctcaagccgcctggtggattcctatgacacagggggcctggaaagaaagggaaagtggatttaaaatatatatatacgtaacccaagccccataacccctaactggacaaatgaggtctgtttctttgggcctgaggctgtgccatataaagtcttattttgggactttacaaacttgtcctaactatcttgtggatagtgggctgtgacaatctggaatagagaacgttcacacttcgctcctttaaagaagcgaccccagatctgcaagggagtagattctgctatcttggcctcacagcccttcctgttgattacaaagcccgtggaagaaaacagaacacaccctcctcagttccgtctaaatgtgtttcttctgcttcaattacaccagttctggggcaaagacactgatgaaacaacacccatacctgaaaagaataaatgtgtgactttcaaatcccctttcgcagtgaaagaaacagcaaacacttaagattcagcatctgttctccagttgcactgaggaatgcactgtctcgcagcaccagctctgcagagcccttgccccagactctttgcggttttatttatatgtatttccatatttcattcctgtgtgtcactgctgcattggtgtggcagcaagtgaccaaatgctacaggtcttactatggacaccaggtcaggtgcaaccacacaaaacaaagccagttccatgagctgcctatgatatgcattgcggaagtaacattttacccagggtgtgccattgcagtgatataaatatatttttttcttagactaaatatgagctgactatctcttttgatgtgtgtacataggtgtgagtgtgtgtgtatgcgtgcctgtctgtgtgcgggtgtgtgtatgtgcatagcctcatgcttaggactacccatgaatgttgtggaatgctacacctggagagttctggttttccaccagtttcaagatgaagaactacatgatacagtggacctggagaccatccccttggaaagacaacccagagatgttcagcatcctgtatctacacgcatcctgtatctacacgtgtattttgtagctgtcacactaaccttaataagaattctacagctttggacagaggcattttcaccttaatggtgaagtaatttaaaatataaatccattcaggtgacaacccatcatcaaaattacaaattttctgattgaactcatctgaatcatcagttccttgatggagagagagaaggagatggaatgtgtctggtaaccccaaatggagtacaagtagcctttgttttcctgcataaatggacttgttgaatgcgaacgaatatatgcaattcatatacttttggagatgaacgtagatatgtgtgtcagctttgagatggtgtgtcctggattaatactttgtctcccaatatcacagaaaaatacatgccagtgactcttgaggttaaggtagttgggatgaaatggcctcaggcaatttcacattccctaattacctggaaagttctacagtaattaatatgcagctaactcctgttgccctcacaagagcatcagccttctagaatcggagctccggagtgtgaagattcagtattgatatgatatgtataccaaactccagccaacttactgccatttttcataatctgagtggctgccttgcttatcctaagctgtggttgcagaaaccgtggccatttatataagctataacatcaaatcagggaaaaatgaggaaaaaaaatagattctgaaccatttattgttgaataagtagagaaaatcatcaataaatatttattacattctgacagggtgtgtggcattgtgttctatgccagagtgacaaagttgattcacccctttttggggaccttaatatattttttaagggatgtgcctatgcattgatgcctgaaaaatatgtataaagaaatgaggttgactcttctgagcagttcatcttttccagaggtaagggtaggaggccaacttcagggtctgggtctgagcccgtgggcaagccctggccgagtgagctccaatgctaactcatgtgccgatctctagagcagtgggaaactaccccgctgcaccaaatcaagtagcttcaccttgtgtatgcaggccccaagttattttttagcaatcttacgagtgaaatgttctggtgggttgaaaaacgttcttattttaaagaaaggttgtgctcgctacactgctggtgtgtgcattctgagacctcttgtattcaatctgtgaaggatatgtgtattaatccgtacacccgtatagcctcaatatttgtctgaagacacttaaattctgacccataaaggaaagttctagaagcaatattttcacttatttaacattctccaaacaacatcaagcattgatacacactgaagagtgcgtttattttttgtatcactctaagtatgttggaatatgcaaggactgtggttcaaattagaatgtataaggcatattataatttagttcatactgaataagaaattaacagaacattgttcggttcacacgttccaaactttgagtgatttctggagttagacatagattttctattttgttttaatttgtcaaggtatttttcttcccttcatgaactttaggtacacataacttatgtcatttatttatggtcttttatacctagtttgtaaaattgtaaaatagcaaactaaatgcaaagagtttgcatttgaaaataataaagtagttgccgtatacaaccctgcaaaaaaaaaaaaaaaaaa

SEQ ID NO: 8 - protein, 986 aa, Homo sapiens
MAGIFYFALFSCLFGICDAVTGSRVYPANEVTLLDSRSVQGELGWIASPLEGGWEEVSIMDEKNTPIRTYQVCNVMEPSQNNWLRTDWITREGAQRVYIEIKFTLRDCNSLPGVMGTCKETFNLYYYESDNDKERFIRENQFVKIDTIAADESFTQVDIGDRIMKLNTEIRDVGPLSKKGFYLAFQDVGACIALVSVRVFYKKCPLTVRNLAQFPDTITGADTSSLVEVRGSCVNNSEEKDVPKMYCGADGEWLVPIGNCLCNAGHEERSGECQACKIGYYKALSTDATCAKCPPHSYSVWEGATSCTCDRGFFRADNDAASMPCTRPPSAPLNLISNVNETSVNLEWSSPQNTGGRQDISYNVVCKKCGAGDPSKCRPCGSGVHYTPQQNGLKTTKVSITDLLAHTNYTFEIWAVNGVSKYNPNPDQSVSVTVTTNQAAPSSIALVQAKEVTRYSVALAWLEPDRPNGVILEYEVKYYEKDQNERSYRIVRTAARNTDIKGLNPLTSYVFHVRARTAAGYGDFSEPLEVTTNTVPSRIIGDGANSTVLLVSVSGSVVLVVILIAAFVISRRRSKYSKAKQEADEEKHLNQGVRTYVDPFTYEDPNQAVREFAKEIDASCIKIEKVIGVGEFGEVCSGRLKVPGKREICVAIKTLKAGYTDKQRRDFLSEASIMGQFDHPNIIHLEGVVTKCKPVMIITEYMENGSLDAFLRKNDGRFTVIQLVGMLRGIGSGMKYLSDMSYVHRDLAARNILVNSNLVCKVSDFGMSRVLEDDPEAAYTTRGGKIPIRWTAPEAIAYRKFTSASDVWSYGIVMWEVMSYGERPYWDMSNQDVIKAIEEGYRLPPPMDCPIALHQLMLDCWQKERSDRPKFGQIVNMLDKLIRNPNSLKRTGTESSRPNTALLDPSSPEFSAVVSVGDWLQAIKMDRYKDNFTAAGYTTLEAVVHVNQEDLARIGITAITHQNKILSSVQAMRTQMQQMHGRMVPV

SEQ ID NO: 9 - DNA, 5229 bp, Homo sapiens
  Inventor MIYAKE, Masato; FUJITA, Yoshiji
gcagtcggagacttgcaggcagcaaacacggtgcgagcgaacaggagtgggggggaaattaaaaaaagctaaacgtggagcagccgatcggggaccgagaaggggaatcgatgcaaggagcacaataaaacaaaagctacttcggaacaaacagcatttaaaaatccacgactcaagataactgaaacctaaaataaaacctgctcatgcaccatggtttttcaaactcggtacccttcatggattattttatgctacatctggctgctccgctttgcacacacaggggaggcgcaggctgcgaaggaagtactactgctggattctaaagcacaacaaacagagttggagtggatttcctctccacccaatgggtgggaagaaattagtggtttggatgagaactataccccgatacgaacataccaggtgtgccaagtcatggagcccaaccaaaacaactggctgcggactaactggatttccaaaggcaatgcacaaaggatttttgtagaattgaaattcaccctgagggattgtaacagtcttcctggagtactgggaacttgcaaggaaacatttaatttgtactattatgaaacagactatgacactggcaggaatataagagaaaacctctatgtaaaaatagacaccattgctgcagatgaaagttttacccaaggtgaccttggtgaaagaaagatgaagcttaacactgaggtgagagagattggacctttgtccaaaaagggattctatcttgcctttcaggatgtaggggcttgcatagctttggtttctgtcaaagtgtactacaagaagtgctggtccattattgagaacttagctatctttccagatacagtgactggttcagaattttcctctttagtcgaggttcgagggacatgtgtcagcagtgcagaggaagaagcggaaaacgcccccaggatgcactgcagtgcagaaggagaatggttagtgcccattggaaaatgtatctgcaaagcaggctaccagcaaaaaggagacacttgtgaaccctgtggccgtgggttctacaagtcttcctctcaagatcttcagtgctctcgttgtccaactcacagtttttctgataaagaaggctcctccagatgtgaatgtgaagatgggtattacagggctccatctgacccaccatacgttgcatgcacaaggcctccatctgcaccacagaacctcattttcaacatcaaccaaaccacagtaagtttggaatggagtcctcctgcagacaatgggggaagaaacgatgtgacctacagaatattgtgtaagcggtgcagttgggagcagggcgaatgtgttccctgtgggagtaacattggatacatgccccagcagactggattagaggataactatgtcactgtcatggacctgctagcccacgctaattatacttttgaagttgaagctgtaaatggagtttctgacttaagccgatcccagaggctctttgctgctgtcagtatcaccactggtcaagcagctccctcgcaagtgagcggagtaatgaaggagagagtactgcagcggagtgtcgagctttcctggcaggaaccagagcatcccaatggagtcatcacagaatatgaaatcaagtattacgagaaagatcaaagggaacggacctactcaacagtaaaaaccaagtctacttcagcctccattaataatctgaaaccaggaacagtgtatgttttccagattcgggcttttactgctgctggttatggaaattacagtcccagacttgatgttgctacactagaggaagctacaggtaaaatgtttgaagctacagctgtctccagtgaacagaatcctgttattatcattgctgtggttgctgtagctgggaccatcattttggtgttcatggtctttggcttcatcattgggagaaggcactgtggttatagcaaagctgaccaagaaggcgatgaagagctttactttcattttaaatttccaggcaccaaaacctacattgaccctgaaacctatgaggacccaaatagagctgtccatcaattcgccaaggagctagatgcctcctgtattaaaattgagcgtgtgattggtgcaggagaattcggtgaagtctgcagtggccgtttgaaacttccagggaaaagagatgttgcagtagccataaaaaccctgaaagttggttacacagaaaaacaaaggagagactttttgtgtgaagcaagcatcatggggcagtttgaccacccaaatgttgtccatttggaaggggttgttacaagagggaaaccagtcatgatagtaatagagttcatggaaaatggagccctagatgcatttctcaggaaacatgatgggcaatttacagtcattcagttagtaggaatgctgagaggaattgctgctggaatgagatatttggctgatatgggatatgttcacagggaccttgcagctcgcaatattcttgtcaacagcaatctcgtttgtaaagtgtcagattttggcctgtcccgagttatagaggatgatccagaagctgtctatacaactactggtggaaaaattccagtaaggtggacagcacccgaagccatccagtaccggaaattcacatcagccagtgatgtatggagctatggaatagtcatgtgggaagttatgtcttatggagaaagaccttattgggacatgtcaaatcaagatgttataaaagcaatagaagaaggttatcgtttaccagcacccatggactgcccagctggccttcaccagctaatgttggattgttggcaaaaggagcgtgctgaaaggccaaaatttgaacagatagttggaattctagacaaaatgattcgaaacccaaatagtctgaaaactcccctgggaacttgtagtaggccaataagccctcttctggatcaaaacactcctgatttcactaccttttgttcagttggagaatggctacaagctattaagatggaaagatataaagataatttcacggcagctggctacaattcccttgaatcagtagccaggatgactattgaggatgtgatgagtttagggatcacactggttggtcatcaaaagaaaatcatgagcagcattcagactatgagagcacaaatgctacatttacatggaactggcattcaagtgtgatatgcatttctcccttttaagggagattacagactgcaagagaacagtactggccttcagtatatgcatagaatgctgctagaagacaagtgatgtcctgggtccttccaacagtgaagagaagatttaagaagcacctatagacttgaactcctaagtgccaccagaatatataaaaagggaatttaggatccaccatcggtggccaggaaaatagcagtgacaataaacaaagtactacctgaaaaacatccaaacaccttgagctctctaacctcctttttgtcttatagactttttaaaatgtacataaagaatttaagaaagaatatatttgtcaaataaaatcatgatcttattgttaaaattaatgaaatattttccttaaatatgtgatttcagactattcctttttaaaatcatttgtgtttattcttcataaggactttgttttagaaagctgtttatagctttggacctttttagtgttaaatctgtaacattactacactgggtacctttgaaagaatctcaaatttcaaaagaaatagcatgattgaagatacatctctgttagaacattggtatcctttttgtgccattttattctgtttaatcagtgctgttttgatattgtttgctaattggcaggtagtcaagaaaatgcaagttgccaagagctctgatattttttaaaaagaatttttttgtaaagatcagacaacacactatcttttcaatgaaaaaagcaataatgatccatacatactataaggcacttttaacagattgtttatagagtgattttactagaaagaatttaataaactcgaagtttaggtttatgagtatataaacaaatgaggcacttcatctgaagaatgttggtgaaggcaagtctctgaaagcagaactatccagtgttatctaaaaattaatctgagcacatcaagattttttcattctcgtgacattaggaaatttaggataaatagttgacatatattttatatcctcttctgttgaatgcagtccaaacatgaaaggaaataattgttttatattataactctgaagcatgataaaggggcagttcacaattttcaccatttaaacacaaatttgctgcacagaatatcaccattgcagttcaaaacaaaacaaaacaaaaagtcttttgtttgtgaacactgatgcaagaaacttgttaaatgaaaggactctttaccctagaaggaagaggtgaaggatctggcttgtttttaaagctttatttattaaaccatattatttgattactgtgttagaatttcataagcaataattaaatgtgtctttatagatattgcaggaatgtatacatattgtgattaatgctttcaaaacttatgaaaatcatgaactaccccagaattgaactgttgtacttccaaagagaattgggctgtttataatgattttaatagagaaagatcccagggatcggtcataattggtcttgtttgataatgtgggcatccacaaacaaacaaacaaataacagaaacaaaatctgtaaatgttcctttgtaaaacttgtaaattttatttatactgtcttgttttgtacacacatttctctgtagtgggctctgaatacattgaaaatgcactatatttttctattttacttgcagagcatcacaaaagaacaggtattttcagtgctacataatgtgttttcccacatttaggaccaaagacggctatagaaaaactcaaatggattgcttcccaaacccctccccaccctttttttttggttttaaatcactgtacagtgttatttgatattttaatttattttttgattgactagaaaaatcattttaatttcactaaaatgttttttgtccctaaggaaaagtaatctgtaaaaataattttaattagcataatacagtcacctagacacttccatttgtaatctttgtaatagactgtaaatatatttttggaactataaaaaaaaaaaaaaaa

SEQ ID NO: 10 - protein, 998 aa, Homo sapiens
MVFQTRYPSWIILCYIWLLRFAHTGEAQAAKEVLLLDSKAQQTELEWISSPPNGWEEISGLDENYTPIRTYQVCQVMEPNQNNWLRTNWISKGNAQRIFVELKFTLRDCNSLPGVLGTCKETFNLYYYETDYDTGRNIRENLYVKIDTIAADESFTQGDLGERKMKLNTEVREIGPLSKKGFYLAFQDVGACIALVSVKVYYKKCWSIIENLAIFPDTVTGSEFSSLVEVRGTCVSSAEEEAENAPRMHCSAEGEWLVPIGKCICKAGYQQKGDTCEPCGRGFYKSSSQDLQCSRCPTHSFSDKEGSSRCECEDGYYRAPSDPPYVACTRPPSAPQNLIFNINQTTVSLEWSPPADNGGRNDVTYRILCKRCSWEQGECVPCGSNIGYMPQQTGLEDNYVTVMDLLAHANYTFEVEAVNGVSDLSRSQRLFAAVSITTGQAAPSQVSGVMKERVLQRSVELSWQEPEHPNGVITEYEIKYYEKDQRERTYSTVKTKSTSASINNLKPGTVYVFQIRAFTAAGYGNYSPRLDVATLEEATGKMFEATAVSSEQNPVIIIAVVAVAGTIILVFMVFGFIIGRRHCGYSKADQEGDEELYFHFKFPGTKTYIDPETYEDPNRAVHQFAKELDASCIKIERVIGAGEFGEVCSGRLKLPGKRDVAVAIKTLKVGYTEKQRRDFLCEASIMGQFDHPNVVHLEGVVTRGKPVMIVIEFMENGALDAFLRKHDGQFTVIQLVGMLRGIAAGMRYLADMGYVHRDLAARNILVNSNLVCKVSDFGLSRVIEDDPEAVYTTTGGKIPVRWTAPEAIQYRKFTSASDVWSYGIVMWEVMSYGERPYWDMSNQDVIKAIEEGYRLPAPMDCPAGLHQLMLDCWQKERAERPKFEQIVGILDKMIRNPNSLKTPLGTCSRPISPLLDQNTPDFTTFCSVGEWLQAIKMERYKDNFTAAGYNSLESVARMTIEDVMSLGITLVGHQKKIMSSIQTMRAQMLHLHGTGIQV

SEQ ID NO: 11 - DNA, 4996 bp, Homo sapiens
  Inventor MIYAKE, Masato; FUJITA, Yoshiji
gcgccggggtgtgcgcccggccgggtgtgcggagagcgagggagcgcgctccctcccgacgcgcgggccgcagcggccaagcccgagggtgcgtggcgcccccgcccgcccggcccggcccggccatggcccccgcccggggccgcctgccccctgcgctctgggtcgtcacggccgcggcggcggcggccacctgcgtgtccgcggcgcgcggcgaagtgaatttgctggacacgtcgaccatccacggggactggggctggctcacgtatccggctcatgggtgggactccatcaacgaggtggacgagtccttccagcccatccacacgtaccaggtttgcaacgtcatgagccccaaccagaacaactggctgcgcacgagctgggtcccccgagacggcgcccggcgcgtctatgctgagatcaagtttaccctgcgcgactgcaacagcatgcctggtgtgctgggcacctgcaaggagaccttcaacctctactacctggagtcggaccgcgacctgggggccagcacacaagaaagccagttcctcaaaatcgacaccattgcggccgacgagagcttcacaggtgccgaccttggtgtgcggcgtctcaagctcaacacggaggtgcgcagtgtgggtcccctcagcaagcgcggcttctacctggccttccaggacataggtgcctgcctggccatcctctctctccgcatctactataagaagtgccctgccatggtgcgcaatctggctgccttctcggaggcagtgacgggggccgactcgtcctcactggtggaggtgaggggccagtgcgtgcggcactcagaggagcgggacacacccaagatgtactgcagcgcggagggcgagtggctcgtgcccatcggcaaatgcgtgtgcagtgccggctacgaggagcggcgggatgcctgtgtggcctgtgagctgggcttctacaagtcagcccctggggaccagctgtgtgcccgctgccctccccacagccactccgcagctccagccgcccaagcctgccactgtgacctcagctactaccgtgcagccctggacccgccgtcctcagcctgcacccggccaccctcggcaccagtgaacctgatctccagtgtgaatgggacatcagtgactctggagtgggcccctcccctggacccaggtggccgcagtgacatcacctacaatgccgtgtgccgccgctgcccctgggcactgagccgctgcgaggcatgtgggagcggcacccgctttgtgccccagcagacaagcctggtgcaggccagcctgctggtggccaacctgctggcccacatgaactactccttctggatcgaggccgtcaatggcgtgtccgacctgagccccgagccccgccgggccgctgtggtcaacatcaccacgaaccaggcagccccgtcccaggtggtggtgatccgtcaagagcgggcggggcagaccagcgtctcgctgctgtggcaggagcccgagcagccgaacggcatcatcctggagtatgagatcaagtactacgagaaggacaaggagatgcagagctactccaccctcaaggccgtcaccaccagagccaccgtctccggcctcaagccgggcacccgctacgtgttccaggtccgagcccgcacctcagcaggctgtggccgcttcagccaggccatggaggtggagaccgggaaaccccggccccgctatgacaccaggaccattgtctggatctgcctgacgctcatcacgggcctggtggtgcttctgctcctgctcatctgcaagaagaggcactgtggctacagcaaggccttccaggactcggacgaggagaagatgcactatcagaatggacaggcacccccacctgtcttcctgcctctgcatcaccccccgggaaagctcccagagccccagttctatgcggaaccccacacctacgaggagccaggccgggcgggccgcagtttcactcgggagatcgaggcctctaggatccacatcgagaaaatcatcggctctggagactccggggaagtctgctacgggaggctgcgggtgccagggcagcgggatgtgcccgtggccatcaaggccctcaaagccggctacacggagagacagaggcgggacttcctgagcgaggcgtccatcatggggcaattcgaccatcccaacatcatccgcctcgagggtgtcgtcacccgtggccgcctggcaatgattgtgactgagtacatggagaacggctctctggacaccttcctgaggacccacgacgggcagttcaccatcatgcagctggtgggcatgctgagaggagtgggtgccggcatgcgctacctctcagacctgggctatgtccaccgagacctggccgcccgcaacgtcctggttgacagcaacctggtctgcaaggtgtctgacttcgggctctcacgggtgctggaggacgacccggatgctgcctacaccaccacgggcgggaagatccccatccgctggacggccccagaggccatcgccttccgcaccttctcctcggccagcgacgtgtggagcttcggcgtggtcatgtgggaggtgctggcctatggggagcggccctactggaacatgaccaaccgggatgtcatcagctctgtggaggaggggtaccgcctgcccgcacccatgggctgcccccacgccctgcaccagctcatgctcgactgttggcacaaggaccgggcgcagcggcctcgcttctcccagattgtcagtgtcctcgatgcgctcatccgcagccctgagagtctcagggccaccgccacagtcagcaggtgcccaccccctgccttcgtccggagctgctttgacctccgagggggcagcggtggcggtgggggcctcaccgtgggggactggctggactccatccgcatgggccggtaccgagaccacttcgctgcgggcggatactcctctctgggcatggtgctacgcatgaacgcccaggacgtgcgcgccctgggcatcaccctcatgggccaccagaagaagatcctgggcagcattcagaccatgcgggcccagctgaccagcacccaggggccccgccggcacctctgatgtacagccagcagggcccaggcagccaccaagcccaccccaggtcatgccagcggcagaggacgtgaggggctggcagcaggcagggcggccccaggcctctgccctcctctcaggtgctggaggagctgaaggcttcgccacaggacctggagttatcaggggtcaggcgcctgggaaggggcctttggtggccaccctggtgaggacacctgtcccccagggcaggcaccttctcttttccagagcctggggcctccacgtcacagagtccaacagggacatcactcgcctgcctctgtgtgcgtgcatgtgtgtgtgtggtggggggtgttctcacaaggtcatgggatctcatgtgaacagtgtgtgatcaagtgtgtccaccccttcgggtctcagcatggacgtgtgcatgttatgagcgtgtgcttatccgttaaggctggaggcacatgtgggtgatggtggatgatgtgtcatgaatgaggaggtgtgtgagcagggaactcagtgtgacaccgccaggtccagcacccatgggggcgggggagaggctcacccctacgtccccccacacctggaggctggagccaggggccacttctgaactgcaccagcaccaggcccaccctcgtctctgcctgggtgagcccaccccggctgtatctcaggtctgggtcctccctctagccgaggaggccacctgcagcctccacccggctctcaccgctgcttcaacaggaaaacagggttcccggtcagtccggctggccgccttcatggaggcatcatggcagagcacatgagatgtcctcagctgggcttggctgcctggccagggccgggggctcagcagcctcctctagcctctgatgccttccctccacggcccaggtctcctcactcaaagtcccttcgccaacctttcaatgcccagccctgacacctgccctttgtcccccaggcctaggatcagggaccagaggatcctatcttctcagcacccagcccaccccttcctgtagcatatggggagactaaggcctggagagaggggtgatgccccgtcccaggttgcactgcaaccaagtgtcagagtcggggctccggcctcctgccaaggctcttgtccccatacaccatcccacaagggcgctgggggtggaagtgccctggaagcccctcccctctcacactgacctccccccttacggcccaccagggtatgtaaatatctcttttctaccatgtcagaatattttttcctcactcctgacaatgcaaaaatggtcttcaaagcacataaaaagcacccagggtgagaaagccccatcccgggggccgttggcaggcagggaagcaggaaccccaccgtgtgccccctgccagccccagagggagtggcgagcccagctgcccagccctgccccccctccccatagccagcacagctatcccgcggggacaccagcactgagccccctctccctcctgcaataattcggggagtctcagccccatccaggtgccgcggccagctctctacacctctatatattatattactatatagccgagctgttcttccttcctatggaagtcggaaacatggtcagaacacgatctgggggggggatcctgtcttcctccccaccccaccccactcttacccaatttctgggctctggatcctcacagtcatggaggcaccgtgggcctggcacttgcaaaagtgtggcccctcactctagtgtgtggtccctctcagggtcctggggatctgcctctctgtggtctccatcctgactcttgaacttacccacaataagaataaattctgcctcatcttt

SEQ ID NO: 12 - protein, 1005 aa, Homo sapiens
MAPARGRLPPALWVVTAAAAAATCVSAARGEVNLLDTSTIHGDWGWLTYPAHGWDSINEVDESFQPIHTYQVCNVMSPNQNNWLRTSWVPRDGARRVYAEIKFTLRDCNSMPGVLGTCKETFNLYYLESDRDLGASTQESQFLKIDTIAADESFTGADLGVRRLKLNTEVRSVGPLSKRGFYLAFQDIGACLAILSLRIYYKKCPAMVRNLAAFSEAVTGADSSSLVEVRGQCVRHSEERDTPKMYCSAEGEWLVPIGKCVCSAGYEERRDACVACELGFYKSAPGDQLCARCPPHSHSAAPAAQACHCDLSYYRAALDPPSSACTRPPSAPVNLISSVNGTSVTLEWAPPLDPGGRSDITYNAVCRRCPWALSRCEACGSGTRFVPQQTSLVQASLLVANLLAHMNYSFWIEAVNGVSDLSPEPRRAAVVNITTNQAAPSQVVVIRQERAGQTSVSLLWQEPEQPNGIILEYEIKYYEKDKEMQSYSTLKAVTTRATVSGLKPGTRYVFQVRARTSAGCGRFSQAMEVETGKPRPRYDTRTIVWICLTLITGLVVLLLLLICKKRHCGYSKAFQDSDEEKMHYQNGQAPPPVFLPLHHPPGKLPEPQFYAEPHTYEEPGRAGRSFTREIEASRIHIEKIIGSGDSGEVCYGRLRVPGQRDVPVAIKALKAGYTERQRRDFLSEASIMGQFDHPNIIRLEGVVTRGRLAMIVTEYMENGSLDTFLRTHDGQFTIMQLVGMLRGVGAGMRYLSDLGYVHRDLAARNVLVDSNLVCKVSDFGLSRVLEDDPDAAYTTTGGKIPIRWTAPEAIAFRTFSSASDVWSFGVVMWEVLAYGERPYWNMTNRDVISSVEEGYRLPAPMGCPHALHQLMLDCWHKDRAQRPRFSQIVSVLDALIRSPESLRATATVSRCPPPAFVRSCFDLRGGSGGGGGLTVGDWLDSIRMGRYRDHFAAGGYSSLGMVLRMNAQDVRALGITLMGHQKKILGSIQTMRAQLTSTQGPRRHL

SEQ ID NO: 13 - DNA, 4536 bp, Homo sapiens
  Inventor MIYAKE, Masato; FUJITA, Yoshiji
gaattccacatgcacacccacacccacgcgcgcccgcaccgccccacgcgcacacactcctgcccacgcccacgcagcgctccgggaagtccggtccgggcgagagcgcgaaaggataccgagaagccacccgcggagagcgcagcggcgccctgggacgcggcgctctcccggcgctgctgcctcggcttggtctcggcctgcgggccgtcggccggcgatggccctggattatctactactgctcctcctggcatccgcagtggctgcgatggaagaaacgttaatggacaccagaacggctactgcagagctgggctggacggccaatcctgcgtccgggtgggaagaagtcagtggctacgatgaaaacctgaacaccatccgcacctaccaggtgtgcaatgtcttcgagcccaaccagaacaattggctgctcaccaccttcatcaaccggcggggggcccatcgcatctacacagagatgcgcttcactgtgagagactgcagcagcctccctaatgtcccaggatcctgcaaggagaccttcaacttgtattactatgagactgactctgtcattgccaccaagaagtcagccttctggtctgaggccccctacctcaaagtagacaccattgctgcagatgagagcttctcccaggtggactttgggggaaggctgatgaaggtaaacacagaagtcaggagctttgggcctcttactcggaatggtttttacctcgcttttcaggattatggagcctgtatgtctcttctttctgtccgtgtcttcttcaaaaagtgtcccagcattgtgcaaaattttgcagtgtttccagagactatgacaggggcagagagcacatctctggtgattgctcggggcacatgcatccccaacgcagaggaagtggacgtgcccatcaaactctactgcaacggggatggggaatggatggtgcctattgggcgatgcacctgcaagcctggctatgagcctgagaacagcgtggcatgcaaggcttgccctgcagggacattcaaggccagccaggaagctgaaggctgctcccactgcccctccaacagccgctcccctgcagaggcgtctcccatctgcacctgtcggaccggttattaccgagcggactttgaccctccagaagtggcatgcactagcgtcccatcaggtccccgcaatgttatctccatcgtcaatgagacgtccatcattctggagtggcaccctccaagggagacaggtgggcgggatgatgtgacctacaacatcatctgcaaaaagtgccgggcagaccgccggagctgctcccgctgtgacgacaatgtggagtttgtgcccaggcagctgggcctgacggagtgccgcgtctccatcagcagcctgtgggcccacaccccctacacctttgacatccaggccatcaatggagtctccagcaagagtcccttccccccacagcacgtctctgtcaacatcaccacaaaccaagccgccccctccaccgttcccatcatgcaccaagtcagtgccactatgaggagcatcaccttgtcatggccacagccggagcagcccaatggcatcatcctggactatgagatccggtactatgagaaggaacacaatgagttcaactcctccatggccaggagtcagaccaacacagcaaggattgatgggctgcggcctggcatggtatatgtggtacaggtgcgtgcccgcactgttgctggctacggcaagttcagtggcaagatgtgcttccagactctgactgacgatgattacaagtcagagctgagggagcagctgcccctgattgctggctcggcagcggccggggtcgtgttcgttgtgtccttggtggccatctctatcgtctgtagcaggaaacgggcttatagcaaagaggctgtgtacagcgataagctccagcattacagcacaggccgaggctccccagggatgaagatctacattgaccccttcacttacgaggatcccaacgaagctgtccgggagtttgccaaggagattgatgtatcttttgtgaaaattgaagaggtcatcggagcaggggagtttggagaagtgtacaaggggcgtttgaaactgccaggcaagagggaaatctacgtggccatcaagaccctgaaggcagggtactcggagaagcagcgtcgggactttctgagtgaggcgagcatcatgggccagttcgaccatcctaacatcattcgcctggagggtgtggtcaccaagagtcggcctgtcatgatcatcacagagttcatggagaatggtgcattggattctttcctcaggcaaaatgacgggcagttcaccgtgatccagcttgtgggtatgctcaggggcatcgctgctggcatgaagtacctggctgagatgaattatgtgcatcgggacctggctgctaggaacattctggtcaacagtaacctggtgtgcaaggtgtccgactttggcctctcccgctacctccaggatgacacctcagatcccacctacaccagctccttgggagggaagatccctgtgagatggacagctccagaggccatcgcctaccgcaagttcacttcagccagcgacgtttggagctatgggatcgtcatgtgggaagtcatgtcatttggagagagaccctattgggatatgtccaaccaagatgtcatcaatgccatcgagcaggactaccggctgcccccacccatggactgtccagctgctctacaccagctcatgctggactgttggcagaaggaccggaacagccggccccggtttgcggagattgtcaacaccctagataagatgatccggaacccggcaagtctcaagactgtggcaaccatcaccgccgtgccttcccagcccctgctcgaccgctccatcccagacttcacggcctttaccaccgtggatgactggctcagcgccatcaaaatggtccagtacagggacagcttcctcactgctggcttcacctccctccagctggtcacccagatgacatcagaagacctcctgagaataggcatcaccttggcaggccatcagaagaagatcctgaacagcattcattctatgagggtccagataagtcagtcaccaacggcaatggcatgagaactcttgtttcttggggaaggagaggagggaaaaggaccagggtcaagggggaccagaggttgaccactgtggaatgtactggagagactggcttctcagctgaggaatgcatttccatcagtgaagaatcaaccggacctgttgctagcaggcaatctccatttctcagtgacagaagcatgtttgagatgccgtgggaaaccaaatatataataataaaaatataaaaaggtgatgttcaacagaagtgaagacaaaacaatatgcatcaggagaacaagagtaaacccagctcccactctcagtgggctgcagttgcccaaccacaggaagaaagggaaggaggtagagggaagaaacagaagcagtgttccattttcttcctcaccaatgacattcttttcttttctcctttcgtactcctccctgagagtcccctcccttctcccacactcgtttccctttgctcatgactcctgtagggaagtttcttcaaacaaaacccagctcctgagtctccagatgttgttctgtcagttgccaaaggactttgctgaccactgcatggggatccaaccaattcaattaatgtcttcatattgaagaagagatgtaccttcaattgaaaacctcgtttttcttttgtttgcattttctgcaaaaaggaaaaagaaaccacaaattggggaaaaaaaaagaagaaaaacctgtttccgtgtgcaaaagcacacatatgtatgtctgtgttataaaatgactgtgcttgttcgtaacagatgcaaacaagaaagaagaactgggaagtctttgtccctaggaaatccaaaggggctggaatatggtgttggtttggctttctggttggcccaatcggcctattggctcaatgggaagagaggagagggagaaaaataaaatgaaaggaaaaaaaaaagtttgcaaattcagacaggaaacaggtgagtggtttgaattggatgcagtgtgggccatcctggaatgatactgactgattaattattcctgataacatctcaagaaaaggagaaggaaagtgtttctggagaatgttctttcacatcactggaatctgcaattcaagaagtgacaagggagaattcttgctttacctatggactggcttaagccgtgtggcatccgaggaatgtttcaaatgtgtctgtgtttctctttacattccttgttgtacctcattgttcaattcacttttgtaaattccacctaacatttaattattttaaatttctccttttaccttaatctccttgctaattttatctgtctaattaaaaagagcagaagcatgtctgggtttacgtaaaaaaaaaaaaaaaaaa

SEQ ID NO: 14 - protein, 984 aa, Homo sapiens
MALDYLLLLLLASAVAAMEETLMDTRTATAELGWTANPASGWEEVSGYDENLNTIRTYQVCNVFEPNQNNWLLTTFINRRGAHRIYTEMRFTVRDCSSLPNVPGSCKETFNLYYYETDSVIATKKSAFWSEAPYLKVDTIAADESFSQVDFGGRLMKVNTEVRSFGPLTRNGFYLAFQDYGACMSLLSVRVFFKKCPSIVQNFAVFPETMTGAESTSLVIARGTCIPNAEEVDVPIKLYCNGDGEWMVPIGRCTCKPGYEPENSVACKACPAGTFKASQEAEGCSHCPSNSRSPAEASPICTCRTGYYRADFDPPEVACTSVPSGPRNVISIVNETSIILEWHPPRETGGRDDVTYNIICKKCRADRRSCSRCDDNVEFVPRQLGLTECRVSISSLWAHTPYTFDIQAINGVSSKSPFPPQHVSVNITTNQAAPSTVPIMHQVSATMRSITLSWPQPEQPNGIILDYEIRYYEKEHNEFNSSMARSQTNTARIDGLRPGMVYVVQVRARTVAGYGKFSGKMCFQTLTDDDYKSELREQLPLIAGSAAAGVVFVVSLVAISIVCSRKRAYSKEAVYSDKLQHYSTGRGSPGMKIYIDPFTYEDPNEAVREFAKEIDVSFVKIEEVIGAGEFGEVYKGRLKLPGKREIYVAIKTLKAGYSEKQRRDFLSEASIMGQFDHPNIIRLEGVVTKSRPVMIITEFMENGALDSFLRQNDGQFTVIQLVGMLRGIAAGMKYLAEMNYVHRDLAARNILVNSNLVCKVSDFGLSRYLQDDTSDPTYTSSLGGKIPVRWTAPEAIAYRKFTSASDVWSYGIVMWEVMSFGERPYWDMSNQDVINAIEQDYRLPPPMDCPAALHQLMLDCWQKDRNSRPRFAEIVNTLDKMIRNPASLKTVATITAVPSQPLLDRSIPDFTAFTTVDDWLSAIKMVQYRDSFLTAGFTSLQLVTQMTSEDLLRIGITLAGHQKKILNSIHSMRVQISQSPTAMA

SEQ ID NO: 15 - DNA, 4869 bp, Homo sapiens
  Inventor MIYAKE, Masato; FUJITA, Yoshiji
cattctgctggctgcgcggtggcggcggctgtgtgtgcgccgcgccttgccgccccccctggccccccgagcccggggcgcgcgctcccgcccgggccgtccgggccccgcggcgccgcggcccgaggccccgggaagcgcagccatggctctgcggaggctgggggccgcgctgctgctgctgccgctgctcgccgccgtggaagaaacgctaatggactccactacagcgactgctgagctgggctggatggtgcatcctccatcagggtgggaagaggtgagtggctacgatgagaacatgaacacgatccgcacgtaccaggtgtgcaacgtgtttgagtcaagccagaacaactggctacggaccaagtttatccggcgccgtggcgcccaccgcatccacgtggagatgaagttttcggtgcgtgactgcagcagcatccccagcgtgcctggctcctgcaaggagaccttcaacctctattactatgaggctgactttgactcggccaccaagaccttccccaactggatggagaatccatgggtgaaggtggataccattgcagccgacgagagcttctcccaggtggacctgggtggccgcgtcatgaaaatcaacaccgaggtgcggagcttcggacctgtgtcccgcagcggcttctacctggccttccaggactatggcggctgcatgtccctcatcgccgtgcgtgtcttctaccgcaagtgcccccgcatcatccagaatggcgccatcttccaggaaaccctgtcgggggctgagagcacatcgctggtggctgcccggggcagctgcatcgccaatgcggaagaggtggatgtacccatcaagctctactgtaacggggacggcgagtggctggtgcccatcgggcgctgcatgtgcaaagcaggcttcgaggccgttgagaatggcaccgtctgccgaggttgtccatctgggactttcaaggccaaccaaggggatgaggcctgtacccactgtcccatcaacagccggaccacttctgaaggggccaccaactgtgtctgccgcaatggctactacagagcagacctggaccccctggacatgccctgcacaaccatcccctccgcgccccaggctgtgatttccagtgtcaatgagacctccctcatgctggagtggacccctccccgcgactccggaggccgagaggacctcgtctacaacatcatctgcaagagctgtggctcgggccggggtgcctgcacccgctgcggggacaatgtacagtacgcaccacgccagctaggcctgaccgagccacgcatttacatcagtgacctgctggcccacacccagtacaccttcgagatccaggctgtgaacggcgttactgaccagagccccttctcgcctcagttcgcctctgtgaacatcaccaccaaccaggcagctccatcggcagtgtccatcatgcatcaggtgagccgcaccgtggacagcattaccctgtcgtggtcccagccggaccagcccaatggcgtgatcctggactatgagctgcagtactatgagaaggagctcagtgagtacaacgccacagccataaaaagccccaccaacacggtcaccgtgcagggcctcaaagccggcgccatctatgtcttccaggtgcgggcacgcaccgtggcaggctacgggcgctacagcggcaagatgtacttccagaccatgacagaagccgagtaccagacaagcatccaggagaagttgccactcatcatcggctcctcggccgctggcctggtcttcctcattgctgtggttgtcatcgccatcgtgtgtaacagaagacgggggtttgagcgtgctgactcggagtacacggacaagctgcaacactacaccagtggccacatgaccccaggcatgaagatctacatcgatcctttcacctacgaggaccccaacgaggcagtgcgggagtttgccaaggaaattgacatctcctgtgtcaaaattgagcaggtgatcggagcaggggagtttggcgaggtctgcagtggccacctgaagctgccaggcaagagagagatctttgtggccatcaagacgctcaagtcgggctacacggagaagcagcgccgggacttcctgagcgaagcctccatcatgggccagttcgaccatcccaacgtcatccacctggagggtgtcgtgaccaagagcacacctgtgatgatcatcaccgagttcatggagaatggctccctggactcctttctccggcaaaacgatgggcagttcacagtcatccagctggtgggcatgcttcggggcatcgcagctggcatgaagtacctggcagacatgaactatgttcaccgtgacctggctgcccgcaacatcctcgtcaacagcaacctggtctgcaaggtgtcggactttgggctctcacgctttctagaggacgatacctcagaccccacctacaccagtgccctgggcggaaagatccccatccgctggacagccccggaagccatccagtaccggaagttcacctcggccagtgatgtgtggagctacggcattgtcatgtgggaggtgatgtcctatggggagcggccctactgggacatgaccaaccaggatgtaatcaatgccattgagcaggactatcggctgccaccgcccatggactgcccgagcgccctgcaccaactcatgctggactgttggcagaaggaccgcaaccaccggcccaagttcggccaaattgtcaacacgctagacaagatgatccgcaatcccaacagcctcaaagccatggcgcccctctcctctggcatcaacctgccgctgctggaccgcacgatccccgactacaccagctttaacacggtggacgagtggctggaggccatcaagatggggcagtacaaggagagcttcgccaatgccggcttcacctcctttgacgtcgtgtctcagatgatgatggaggacattctccgggttggggtcactttggctggccaccagaaaaaaatcctgaacagtatccaggtgatgcgggcgcagatgaaccagattcagtctgtggaggtttgacattcacctgcctcggctcacctcttcctccaagccccgccccctctgccccacgtgccggccctcctggtgctctatccactgcagggccagccactcgccaggaggccacgggccacgggaagaaccaagcggtgccagccacgagacgtcaccaagaaaacatgcaactcaaacgacggaaaaaaaaagggaatgggaaaaaagaaaacagatcctgggagggggcgggaaatacaaggaatattttttaaagaggattctcataaggaaagcaatgactgttcttgcgggggataaaaaagggcttgggagattcatgcgatgtgtccaatcggagacaaaagcagtttctctccaactccctctgggaaggtgacctggccagagccaagaaacactttcagaaaaacaaatgtgaaggggagagacaggggccgcccttggctcctgtccctgctgctcctctaggcctcactcaacaaccaagcgcctggaggacgggacagatggacagacagccaccctgagaacccctctgggaaaatctattcctgccaccactgggcaaacagaagaatttttctgtctttggagagtattttagaaactccaatgaaagacactgtttctcctgttggctcacagggctgaaaggggcttttgtcctcctgggtcagggagaacgcggggaccccagaaaggtcagccttcctgaggatgggcaacccccaggtctgcagctccaggtacatatcacgcgcacagcctggcagcctggccctcctggtgcccactcccgccagcccctgcctcgaggactgatactgcagtgactgccgtcagctccgactgccgctgagaagggttgatcctgcatctgggtttgtttacagcaattcctggactcgggggtattttggtcacagggtggttttggtttagggggtttgtttgttgggttgttttttgttttttggttttttttaatgacaatgaagtgacactttgacatttcctaccttttgaggacttgatccttctccaggaagaaggtgctttctgcttactgacttaggcaatacaccaagggcgagattttatatgcacatttctggatttttttatacggttttcattgacactcttccctcctcccacctgccaccaggcctcaccaaagcccactgccatggggccatctgggccattcagagactggagtgagatttgggtgtggagggggaggcgccaaggtggaggagcttcccactccaggactgttgatgaaagggacagattgaggaggaagtgggctctgaggctgcagggctggaagtccttgcccacttcccactctcctgccccaatctatctagtacttcccaggcaaataggcccctttgaggctcctgagtgccctcagatggtcaaaacccagttttccctctgggagcctaaaccaggctgcatcggaggccaggacccggatcattcactgtgataccctgccctccagagggtgcgctcagagacacgggcaagcatgcctcttcccttccctggagagaaagtgtgtgatttctctcccacctccttccccccaccagacctttgctgggcctaaaggtcttggccatggggacgccctcagtctagggatctggccacagactccctcctgtgaaccaacacagacacccaagcagagcaatcagttagtgaattgaatggaaataaacgctttagttataatatga

SEQ ID NO: 16 - protein, 987 aa, Homo sapiens
MALRRLGAALLLLPLLAAVEETLMDSTTATAELGWMVHPPSGWEEVSGYDENMNTIRTYQVCNVFESSQNNWLRTKFIRRRGAHRIHVEMKFSVRDCSSIPSVPGSCKETFNLYYYEADFDSATKTFPNWMENPWVKVDTIAADESFSQVDLGGRVMKINTEVRSFGPVSRSGFYLAFQDYGGCMSLIAVRVFYRKCPRIIQNGAIFQETLSGAESTSLVAARGSCIANAEEVDVPIKLYCNGDGEWLVPIGRCMCKAGFEAVENGTVCRGCPSGTFKANQGDEACTHCPINSRTTSEGATNCVCRNGYYRADLDPLDMPCTTIPSAPQAVISSVNETSLMLEWTPPRDSGGREDLVYNIICKSCGSGRGACTRCGDNVQYAPRQLGLTEPRIYISDLLAHTQYTFEIQAVNGVTDQSPFSPQFASVNITTNQAAPSAVSIMHQVSRTVDSITLSWSQPDQPNGVILDYELQYYEKELSEYNATAIKSPTNTVTVQGLKAGAIYVFQVRARTVAGYGRYSGKMYFQTMTEAEYQTSIQEKLPLIIGSSAAGLVFLIAVVVIAIVCNRRRGFERADSEYTDKLQHYTSGHMTPGMKIYIDPFTYEDPNEAVREFAKEIDISCVKIEQVIGAGEFGEVCSGHLKLPGKREIFVAIKTLKSGYTEKQRRDFLSEASIMGQFDHPNVIHLEGVVTKSTPVMIITEFMENGSLDSFLRQNDGQFTVIQLVGMLRGIAAGMKYLADMNYVHRDLAARNILVNSNLVCKVSDFGLSRFLEDDTSDPTYTSALGGKIPIRWTAPEAIQYRKFTSASDVWSYGIVMWEVMSYGERPYWDMTNQDVINAIEQDYRLPPPMDCPSALHQLMLDCWQKDRNHRPKFGQIVNTLDKMIRNPNSLKAMAPLSSGINLPLLDRTIPDYTSFNTVDEWLEAIKMGQYKESFANAGFTSFDVVSQMMMEDILRVGVTLAGHQKKILNSIQVMRAQMNQIQSVEV

SEQ ID NO: 17 - DNA, 4234 bp, Homo sapiens
  Inventor MIYAKE, Masato; FUJITA, Yoshiji
cgtgagcggcgcagcaagatcccagctcggaccccggacggcgcgcgcccccgaagccccggatcccagtcgggcccgcagctgaccgccagattactgtgcatcccgaatcacgaccacctgcaccctcctgccccggcccgccccccaagtcctcaggcacccagctccccggcgccccggatcctcctggaccggtccgtccagattcccgcgggaccgacctgtccgcatccccaggaccgccgggctcggtgcaccgcctcggtcccggagccgcccgcctggattgcattccctcctctcctggatctcctgggacccgacgcgagcctgccccggagcccgccgagcgcaccctctctcgggtgcctgcagccccgccggcgcggcccggcccggcgcggcccggctcggctcctagagctgccacggccatggccagagcccgcccgccgccgccgccgtcgccgccgccggggcttctgccgctgctccctccgctgctgctgctgccgctgctgctgctgcccgccggctgccgggcgctggaagagaccctcatggacacaaaatgggtaacatctgagttggcgtggacatctcatccagaaagtgggtgggaagaggtgagtggctacgatgaggccatgaatcccatccgcacataccaggtgtgtaatgtgcgcgagtcaagccagaacaactggcttcgcacggggttcatctggcggcgggatgtgcagcgggtctacgtggagctcaagttcactgtgcgtgactgcaacagcatccccaacatccccggctcctgcaaggagaccttcaacctcttctactacgaggctgacagcgatgtggcctcagcctcctcccccttctggatggagaacccctacgtgaaagtggacaccattgcacccgatgagagcttctcgcggctggatgccggccgtgtcaacaccaaggtgcgcagctttgggccactttccaaggctggcttctacctggccttccaggaccagggcgcctgcatgtcgctcatctccgtgcgcgccttctacaagaagtgtgcatccaccaccgcaggcttcgcactcttccccgagaccctcactggggcggagcccacctcgctggtcattgctcctggcacctgcatccctaacgccgtggaggtgtcggtgccactcaagctctactgcaacggcgatggggagtggatggtgcctgtgggtgcctgcacctgtgccaccggccatgagccagctgccaaggagtcccagtgccgcccctgtccccctgggagctacaaggcgaagcagggagaggggccctgcctcccatgtccccccaacagccgtaccacctccccagccgccagcatctgcacctgccacaataacttctaccgtgcagactcggactctgcggacagtgcctgtaccaccgtgccatctccaccccgaggtgtgatctccaatgtgaatgaaacctcactgatcctcgagtggagtgagccccgggacctgggtggccgggatgacctcctgtacaatgtcatctgcaagaagtgccatggggctggaggggcctcagcctgctcacgctgtgatgacaacgtggagtttgtgcctcggcagctgggcctgacggagcgccgggtccacatcagccatctgctggcccacacgcgctacacctttgaggtgcaggcggtcaacggtgtctcgggcaagagccctctgccgcctcgttatgcggccgtgaatatcaccacaaaccaggctgccccgtctgaagtgcccacactacgcctgcacagcagctcaggcagcagcctcaccctatcctgggcacccccagagcggcccaacggagtcatcctggactacgagatgaagtactttgagaagagcgagggcatcgcctccacagtgaccagccagatgaactccgtgcagctggacgggcttcggcctgacgcccgctatgtggtccaggtccgtgcccgcacagtagctggctatgggcagtacagccgccctgccgagtttgagaccacaagtgagagaggctctggggcccagcagctccaggagcagcttcccctcatcgtgggctccgctacagctgggcttgtcttcgtggtggctgtcgtggtcatcgctatcgtctgcctcaggaagcagcgacacggctctgattcggagtacacggagaagctgcagcagtacattgctcctggaatgaaggtttatattgacccttttacctacgaggaccctaatgaggctgttcgggagtttgccaaggagatcgacgtgtcctgcgtcaagatcgaggaggtgatcggagctggggaatttggggaagtgtgccgtggtcgactgaaacagcctggccgccgagaggtgtttgtggccatcaagacgctgaaggtgggctacaccgagaggcagcggcgggacttcctaagcgaggcctccatcatgggtcagtttgatcaccccaatataatccggctcgagggcgtggtcaccaaaagtcggccagttatgatcctcactgagttcatggaaaactgcgccctggactccttcctccggctcaacgatgggcagttcacggtcatccagctggtgggcatgttgcggggcattgctgccggcatgaagtacctgtccgagatgaactatgtgcaccgcgacctggctgctcgcaacatccttgtcaacagcaacctggtctgcaaagtctcagactttggcctctcccgcttcctggaggatgacccctccgatcctacctacaccagttccctgggcgggaagatccccatccgctggactgccccagaggccatagcctatcggaagttcacttctgctagtgatgtctggagctacggaattgtcatgtgggaggtcatgagctatggagagcgaccctactgggacatgagcaaccaggatgtcatcaatgccgtggagcaggattaccggctgccaccacccatggactgtcccacagcactgcaccagctcatgctggactgctgggtgcgggaccggaacctcaggcccaaattctcccagattgtcaataccctggacaagctcatccgcaatgctgccagcctcaaggtcattgccagcgctcagtctggcatgtcacagcccctcctggaccgcacggtcccagattacacaaccttcacgacagttggtgattggctggatgccatcaagatggggcggtacaaggagagcttcgtcagtgcggggtttgcatcttttgacctggtggcccagatgacggcagaagacctgctccgtattggggtcaccctggccggccaccagaagaagatcctgagcagtatccaggacatgcggctgcagatgaaccagacgctgcctgtgcaggtctgacaccggctcccacggggaccctgaggaccgtgcagggatgccaagcagccggctggactttcggactcttggacttttggatgcctggccttaggctgtggcccagaagctggaagtttgggaaaggcccaagctgggacttctccaggcctgtgttccctccccaggaagtgcgccccaaacctcttcatattgaagatggattaggagagggggtgatgacccctccccaagcccctcagggcccagaccttcctgctctccagcaggggatccccacaacctcacacttgtctgttcttcagtgctggaggtcctggcagggtcaggctggggtaagccggggttccacagggcccagccctggcaggggtctggccccccaggtaggcggagagcagtccctccctcaggaactggaggaggggactccaggaatggggaaatgtgacaccaccatcctgaagccagcttgcacctccagtttgcacagggatttgttctgggggctgagggccctgtccccacccccgcccttggtgctgtcataaaagggcaggcaggggcaggctgaggagttgccctttgccccccagagactgactctcagagccagagatgggatgtgtgagtgtgtgtgtgtgtgtgtgtgtgtgcgcgcgcgcgcgcgtgtgtgtgtgcacgcactggcctgcacagagagcatgggtgagcgtgtaaaagcttggccctgtgccctacaatggggccagctgggccgacagcagaataaaggcaataagatgaaaaaaaaaaaaaaa

SEQ ID NO: 18 - protein, 998 aa, Homo sapiens
MARARPPPPPSPPPGLLPLLPPLLLLPLLLLPAGCRALEETLMDTKWVTSELAWTSHPESGWEEVSGYDEAMNPIRTYQVCNVRESSQNNWLRTGFIWRRDVQRVYVELKFTVRDCNSIPNIPGSCKETFNLFYYEADSDVASASSPFWMENPYVKVDTIAPDESFSRLDAGRVNTKVRSFGPLSKAGFYLAFQDQGACMSLISVRAFYKKCASTTAGFALFPETLTGAEPTSLVIAPGTCIPNAVEVSVPLKLYCNGDGEWMVPVGACTCATGHEPAAKESQCRPCPPGSYKAKQGEGPCLPCPPNSRTTSPAASICTCHNNFYRADSDSADSACTTVPSPPRGVISNVNETSLILEWSEPRDLGGRDDLLYNVICKKCHGAGGASACSRCDDNVEFVPRQLGLTERRVHISHLLAHTRYTFEVQAVNGVSGKSPLPPRYAAVNITTNQAAPSEVPTLRLHSSSGSSLTLSWAPPERPNGVILDYEMKYFEKSEGIASTVTSQMNSVQLDGLRPDARYVVQVRARTVAGYGQYSRPAEFETTSERGSGAQQLQEQLPLIVGSATAGLVFVVAVVVIAIVCLRKQRHGSDSEYTEKLQQYIAPGMKVYIDPFTYEDPNEAVREFAKEIDVSCVKIEEVIGAGEFGEVCRGRLKQPGRREVFVAIKTLKVGYTERQRRDFLSEASIMGQFDHPNIIRLEGVVTKSRPVMILTEFMENCALDSFLRLNDGQFTVIQLVGMLRGIAAGMKYLSEMNYVHRDLAARNILVNSNLVCKVSDFGLSRFLEDDPSDPTYTSSLGGKIPIRWTAPEAIAYRKFTSASDVWSYGIVMWEVMSYGERPYWDMSNQDVINAVEQDYRLPPPMDCPTALHQLMLDCWVRDRNLRPKFSQIVNTLDKLIRNAASLKVIASAQSGMSQPLLDRTVPDYTTFTTVGDWLDAIKMGRYKESFVSAGFASFDLVAQMTAEDLLRIGVTLAGHQKKILSSIQDMRLQMNQTLPVQV

SEQ ID NO: 19 - DNA, 4369 bp, Homo sapiens
  Inventor MIYAKE, Masato; FUJITA, Yoshiji
ttccagcgcagctcagcccctgcccggcccggcccgcccggctccgcgccgcagtctccctccctcccgctccgtccccgctcgggctcccaccatccccgcccgcgaggagagcactcggcccggcggcgcgagcagagccactccagggagggggggagaccgcgagcggccggctcagcccccgccacccggggcgggaccccgaggccccggagggaccccaactccagccacgtcttgctgcgcgcccgcccggcgcggccactgccagcacgctccgggcccgccgcccgcgcgcgcggcacagacgcggggccacacttggcgccgccgcccggtgccccgcacgctcgcatgggcccgcgctgagggccccgacgaggagtcccgcgcggagtatcggcgtccacccgcccagggagagtcagacctgggggggcgagggccccccaaactcagttcggatcctacccgagtgaggcggcgccatggagctccgggtgctgctctgctgggcttcgttggccgcagctttggaagagaccctgctgaacacaaaattggaaactgctgatctgaagtgggtgacattccctcaggtggacgggcagtgggaggaactgagcggcctggatgaggaacagcacagcgtgcgcacctacgaagtgtgtgacgtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtggacacggtggccgcggagcatctcacccggaagcgccctggggccgaggccaccgggaaggtgaatgtcaagacgctgcgtctgggaccgctcagcaaggctggcttctacctggccttccaggaccagggtgcctgcatggccctgctatccctgcacctcttctacaaaaagtgcgcccagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctccgcggagcgtggtttcccgcctgaacggctcctccctgcacctggaatggagtgcccccctggagtctggtggccgagaggacctcacctacgccctccgctgccgggagtgccgacccggaggctcctgtgcgccctgcgggggagacctgacttttgaccccggcccccgggacctggtggagccctgggtggtggttcgagggctacgtcctgacttcacctatacctttgaggtcactgcattgaacggggtatcctccttagccacggggcccgtcccatttgagcctgtcaatgtcaccactgaccgagaggtacctcctgcagtgtctgacatccgggtgacgcggtcctcacccagcagcttgagcctggcctgggctgttccccgggcacccagtggggctgtgctggactacgaggtcaaataccatgagaagggcgccgagggtcccagcagcgtgcggttcctgaagacgtcagaaaaccgggcagagctgcgggggctgaagcggggagccagctacctggtgcaggtacgggcgcgctctgaggccggctacgggcccttcggccaggaacatcacagccagacccaactggatgagagcgagggctggcgggagcagctggccctgattgcgggcacggcagtcgtgggtgtggtcctggtcctggtggtcattgtggtcgcagttctctgcctcaggaagcagagcaatgggagagaagcagaatattcggacaaacacggacagtatctcatcggacatggtactaaggtctacatcgaccccttcacttatgaagaccctaatgaggctgtgagggaatttgcaaaagagatcgatgtctcctacgtcaagattgaagaggtgattggtgcaggtgagtttggcgaggtgtgccgggggcggctcaaggccccagggaagaaggagagctgtgtggcaatcaagaccctgaagggtggctacacggagcggcagcggcgtgagtttctgagcgaggcctccatcatgggccagttcgagcaccccaatatcatccgcctggagggcgtggtcaccaacagcatgcccgtcatgattctcacagagttcatggagaacggcgccctggactccttcctgcggctaaacgacggacagttcacagtcatccagctcgtgggcatgctgcggggcatcgcctcgggcatgcggtaccttgccgagatgagctacgtccaccgagacctggctgctcgcaacatcctagtcaacagcaacctcgtctgcaaagtgtctgactttggcctttcccgattcctggaggagaactcttccgatcccacctacacgagctccctgggaggaaagattcccatccgatggactgccccggaggccattgccttccggaagttcacttccgccagtgatgcctggagttacgggattgtgatgtgggaggtgatgtcatttggggagaggccgtactgggacatgagcaatcaggacgtgatcaatgccattgaacaggactaccggctgcccccgcccccagactgtcccacctccctccaccagctcatgctggactgttggcagaaagaccggaatgcccggccccgcttcccccaggtggtcagcgccctggacaagatgatccggaaccccgccagcctcaaaatcgtggcccgggagaatggcggggcctcacaccctctcctggaccagcggcagcctcactactcagcttttggctctgtgggcgagtggcttcgggccatcaaaatgggaagatacgaagaaagtttcgcagccgctggctttggctccttcgagctggtcagccagatctctgctgaggacctgctccgaatcggagtcactctggcgggacaccagaagaaaatcttggccagtgtccagcacatgaagtcccaggccaagccgggaaccccgggtgggacaggaggaccggccccgcagtactgacctgcaggaactccccaccccagggacaccgcctccccattttccggggcagagtggggactcacagaggcccccagccctgtgccccgctggattgcactttgagcccgtggggtgaggagttggcaatttggagagacaggatttgggggttctgccataataggaggggaaaatcaccccccagccacctcggggaactccagaccaagggtgagggcgcctttccctcaggactgggtgtgaccagaggaaaaggaagtgcccaacatctcccagcctccccaggtgcccccctcaccttgatgggtgcgttcccgcagaccaaagagagtgtgactcccttgccagctccagagtgggggggctgtcccagggggcaagaaggggtgtcagggcccagtgacaaaatcattggggtttgtagtcccaacttgctgctgtcaccaccaaactcaatcatttttttcccttgtaaatgcccctcccccagctgctgccttcatattgaaggtttttgagttttgtttttggtcttaatttttctccccgttccctttttgtttcttcgttttgtttttctaccgtccttgtcataactttgtgttggagggaacctgtttcactatggcctcctttgcccaagttgaaacaggggcccatcatcatgtctgtttccagaacagtgccttggtcatcccacatccccggaccccgcctgggacccccaagctgtgtcctatgaaggggtgtggggtgaggtagtgaaaagggcggtagttggtggtggaacccagaaacggacgccggtgcttggaggggttcttaaattatatttaaaaaagtaactttttgtataaataaaagaaaatgggacgtgtcccagctccaggggtaaaaaaaaaaaaaaaaaa

SEQ ID NO: 20 - protein, 987 aa, Homo sapiens
MELRVLLCWASLAAALEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQLALIAGTAVVGVVLVLVVIVVAVLCLRKQSNGREAEYSDKHGQYLIGHGTKVYIDPFTYEDPNEAVREFAKEIDVSYVKIEEVIGAGEFGEVCRGRLKAPGKKESCVAIKTLKGGYTERQRREFLSEASIMGQFEHPNIIRLEGVVTNSMPVMILTEFMENGALDSFLRLNDGQFTVIQLVGMLRGIASGMRYLAEMSYVHRDLAARNILVNSNLVCKVSDFGLSRFLEENSSDPTYTSSLGGKIPIRWTAPEAIAFRKFTSASDAWSYGIVMWEVMSFGERPYWDMSNQDVINAIEQDYRLPPPPDCPTSLHQLMLDCWQKDRNARPRFPQVVSALDKMIRNPASLKIVARENGGASHPLLDQRQPHYSAFGSVGEWLRAIKMGRYEESFAAAGFGSFELVSQISAEDLLRIGVTLAGHQKKILASVQHMKSQAKPGTPGGTGGPAPQY

SEQ ID NO: 21 - DNA, 4059 bp, Homo sapiens
  Inventor MIYAKE, Masato; FUJITA, Yoshiji
ggggcgggccgggccggcttgggaggggacgcggagggggcgggccgggctgcgttcgctccagccgcggctctacagcagcgggcggcgggacccgggacccagcttggcgacggcgatctcgacgcgggcccccaggatctcccggcgccccacctctggagcagcccctgccgccagcgtcaggtccaccccggaatcccagggactctcggcgccgaacggacccgggccgggtgcaacggggtccccggactggagaagacgcgggtggcaccgtgcgagctccaggagccccgggtccactgcgaggcctcggggggcgcagacctgcagagactgcggccaacgggaagaaataaagggattatagtccacccaattcacagacttctgagactcagacacgaggagagatagagaaccgccaatctctagatcaacaagcaaaggaggtgccaagcctgtttgtcttcattgtgacactggagtctagatgctgggaagtccaagatcagggtgccggcatggtcagttcctggcgaagcctctcttctaggtttcagactgccctcttctttgttgtgtcctcgaatggcagaaaaaggggtggctgttggaggaagggaggagagtaaatgaagagaaagaactggaataaccccttgcagaaaaaaaaaaaaagggaagcaagcttagctgtacaccctgagtcttgcaaaagctgcagccccacccaggagcagggtggtggctggggcgatggtggacgccctgaagatgtcccatggctactgaaggggctgcccagttagggaacagagtggcgggcatggtgtgtagcctatgggtgctgctcctggtgtcttcagttctggctctggaagaggtattgctggacaccaccggagagacatctgagattggctggctcacctacccaccaggggggtgggacgaggtgagtgttctggacgaccagcgacgcctgactcggacctttgaggcatgtcatgtggcaggggcccctccaggcaccgggcaggacaattggttgcagacacactttgtggagcggcgcggggcccagagggcgcacattcgactccacttctctgtgcgggcatgctccagcctgggtgtgagcggcggcacctgccgggagaccttcaccctttactaccgtcaggctgaggagcccgacagccctgacagcgtttcctcctggcacctcaaacgctggaccaaggtggacacaattgcagcagacgagagctttccctcctcctcctcctcctcctcctcttcttcctctgcagcgtgggctgtgggaccccacggggctgggcagcgggctggactgcaactgaacgtcaaagagcggagctttgggcctctcacccaacgcggcttctacgtggccttccaggacacgggggcctgcctggccctggtcgctgtcaggctcttctcctacacctgccctgccgtgctccgatcctttgcttcctttccagagacgcaggccagtggggctgggggggcctccctggtggcagctgtgggcacctgtgtggctcatgcagagccagaggaggatggagtagggggccaggcaggaggcagcccccccaggctgcactgcaacggggagggcaagtggatggtagctgtcgggggctgccgctgccagcctggataccaaccagcacgaggagacaaggcctgccaagcctgcccacgggggctctataagtcttctgctgggaatgctccctgctcaccatgccctgcccgcagtcacgctcccaacccagcagcccccgtttgcccctgcctggagggcttctaccgggccagttccgacccaccagaggccccctgcactggtcctccatcggctccccaggagctttggtttgaggtgcaaggctcagcactcatgctacactggcgcctgcctcgggagctggggggtcgaggggacctgctcttcaatgtcgtgtgcaaggagtgtgaaggccgccaggaacctgccagcggtggtgggggcacttgtcaccgctgcagggatgaggtccacttcgaccctcgccagagaggcctgactgagagccgagtgttagtggggggactccgggcacacgtaccctacatcttagaggtgcaggctgttaatggggtgtctgagctcagccctgaccctcctcaggctgcagccatcaatgtcagcaccagccatgaagtgccctctgctgtccctgtggtgcaccaggtgagccgggcatccaacagcatcacggtgtcctggccgcagcccgaccagaccaatgggaacatcctggactatcagctccgctactatgaccaggcagaagacgaatcccactccttcaccctgaccagcgagaccaacactgccaccgtgacacagctgagccctggccacatctatggtttccaggtgcgggcccggactgctgccggccacggcccctacgggggcaaagtctatttccagacacttcctcaaggggagctgtcttcccagcttccggaaagactctccttggtgatcggctccatcctgggggctttggccttcctcctgctggcagccatcaccgtgctggcggtcgtcttccagcggaagcggcgtgggactggctacacggagcagctgcagcaatacagcagcccaggactcggggtgaagtattacatcgacccctccacctacgaggacccctgtcaggccatccgagaacttgcccgggaagtcgatcctgcttatatcaagattgaggaggtcattgggacaggctcttttggagaagtgcgccagggccgcctgcagccacggggacggagggagcagactgtggccatccaggccctgtgggccgggggcgccgaaagcctgcagatgaccttcctgggccgggccgcagtgctgggtcagttccagcaccccaacatcctgcggctggagggcgtggtcaccaagagccgacccctcatggtgctgacggagttcatggagcttggccccctggacagcttcctcaggcagcgggagggccagttcagcagcctgcagctggtggccatgcagcggggagtggctgctgccatgcagtacctgtccagctttgccttcgtccatcgctcgctgtctgcccacagcgtgctggtgaatagccacttggtgtgcaaggtggcccgtcttggccacagtcctcagggcccaagttgtttgcttcgctgggcagccccagaggtcattgcacatggaaagcatacaacatccagtgatgtctggagctttgggatactcatgtgggaagtgatgagttatggagaacggccttactgggacatgagtgagcaggaggtactaaatgcaatagagcaggagttccggctgcccccgcctccaggctgtcctcctggattacatctacttatgttggacacttggcagaaggaccgtgcccggcggcctcattttgaccagctggtggctgcatttgacaagatgatccgcaagccagataccctgcaggctggcggggacccaggggaaaggccttcccaggcccttctgacccctgtggccctggactttccttgtctggactcaccccaggcctggctttcagccattggactggagtgctaccaggacaacttctccaagtttggcctctgtaccttcagtgatgtggctcagctcagcctagaagacctgcctgccctgggcatcaccctggctggccaccagaagaagctgctgcaccacatccagctccttcagcaacacctgaggcagcagggctcagtggaggtctgagaatgacgatacccgtgactcagccctggacactggtccgagaagggacatgtgggacgtgagccgggctccaacagcctctgtgagagatgccccacaccaaacccaaccctcccgatggctgcattccctggtcctccgcctctccaccagccccctcctcattaaagggaaagaagggaatttgcaaaaaaaaaaaaaaaaaa

SEQ ID NO: 22 - protein, 1006 aa, Homo sapiens
MVCSLWVLLLVSSVLALEEVLLDTTGETSEIGWLTYPPGGWDEVSVLDDQRRLTRTFEACHVAGAPPGTGQDNWLQTHFVERRGAQRAHIRLHFSVRACSSLGVSGGTCRETFTLYYRQAEEPDSPDSVSSWHLKRWTKVDTIAADESFPSSSSSSSSSSSAAWAVGPHGAGQRAGLQLNVKERSFGPLTQRGFYVAFQDTGACLALVAVRLFSYTCPAVLRSFASFPETQASGAGGASLVAAVGTCVAHAEPEEDGVGGQAGGSPPRLHCNGEGKWMVAVGGCRCQPGYQPARGDKACQACPRGLYKSSAGNAPCSPCPARSHAPNPAAPVCPCLEGFYRASSDPPEAPCTGPPSAPQELWFEVQGSALMLHWRLPRELGGRGDLLFNVVCKECEGRQEPASGGGGTCHRCRDEVHFDPRQRGLTESRVLVGGLRAHVPYILEVQAVNGVSELSPDPPQAAAINVSTSHEVPSAVPVVHQVSRASNSITVSWPQPDQTNGNILDYQLRYYDQAEDESHSFTLTSETNTATVTQLSPGHIYGFQVRARTAAGHGPYGGKVYFQTLPQGELSSQLPERLSLVIGSILGALAFLLLAAITVLAVVFQRKRRGTGYTEQLQQYSSPGLGVKYYIDPSTYEDPCQAIRELAREVDPAYIKIEEVIGTGSFGEVRQGRLQPRGRREQTVAIQALWAGGAESLQMTFLGRAAVLGQFQHPNILRLEGVVTKSRPLMVLTEFMELGPLDSFLRQREGQFSSLQLVAMQRGVAAAMQYLSSFAFVHRSLSAHSVLVNSHLVCKVARLGHSPQGPSCLLRWAAPEVIAHGKHTTSSDVWSFGILMWEVMSYGERPYWDMSEQEVLNAIEQEFRLPPPPGCPPGLHLLMLDTWQKDRARRPHFDQLVAAFDKMIRKPDTLQAGGDPGERPSQALLTPVALDFPCLDSPQAWLSAIGLECYQDNFSKFGLCTFSDVAQLSLEDLPALGITLAGHQKKLLHHIQLLQQHLRQQGSVEV

SEQ ID NO: 23 - DNA, 5084 bp, Homo sapiens
  Inventor MIYAKE, Masato; FUJITA, Yoshiji
gatcccatcgcagctaccgcgatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctcttcttttggcttccatgatggttattttcttttctttcaacttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatctagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgaaacatttaaattttaccctttagactgtagcctggatattattcttgtagtttacctctttaaaaacaaaacaaaacaaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttgtgttgtcacccaagagattgttgtttgccatactttgtctgaaaaattcctttgtgtttctattgacttcaatgatagtaagaaaagtggttgttagttatagatgtctaggtacttcaggggcacttcattgagagttttgtcttgccatactttgtctgaaaaattcctttgtgtttctattgacttcaatgatagtaagaaaagtggttgttagttatagatgtctaggtacttcaggggcacttcattgagagttttgtcaatgtcttttgaatattcccaagcccatgagtccttgaaaatattttttatatatacagtaactttatgtgtaaatacataagcggcgtaagtttaaaggatgttggtgttccacgtgttttattcctgtatgttgtccaattgttgacagttctgaagaattc

SEQ ID NO: 24 - protein, 976 aa, Homo sapiens [sequence omitted]

SEQ ID NO: 25 - DNA, 6222 bp, Homo sapiens
  Inventor MIYAKE, Masato; FUJITA, Yoshiji
gggggcggcagcggtggtagcagctggtacctcccgccgcctctgttcggagggtcgcggggcaccgaggtgctttccggccgccctctggtcggccacccaaagccgcgggcgctgatgatgggtgaggagggggcggcaagatttcgggcgcccctgccctgaacgccctcagctgctgccgccggggccgctccagtgcctgcgaactctgaggagccgaggcgccggtgagagcaaggacgctgcaaacttgcgcagcgcgggggctgggattcacgcccagaagttcagcaggcagacagtccgaagccttcccgcagcggagagatagcttgagggtgcgcaagacggcagcctccgccctcggttcccgcccagaccgggcagaagagcttggaggagccaaaaggaacgcaaaaggcggccaggacagcgtgcagcagctgggagccgccgttctcagccttaaaagttgcagagattggaggctgccccgagaggggacagaccccagctccgactgcggggggcaggagaggacggtacccaactgccacctcccttcaaccatagtagttcctctgtaccgagcgcagcgagctacagacgggggcgcggcactcggcgcggagagcgggaggctcaaggtcccagccagtgagcccagtgtgcttgagtgtctctggactcgcccctgagcttccaggtctgtttcatttagactcctgctcgcctccgtgcagttgggggaaagcaagagacttgcgcgcacgcacagtcctctggagatcaggtggaaggagccgctgggtaccaaggactgttcagagcctcttcccatctcggggagagcgaagggtgaggctgggcccggagagcagtgtaaacggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaaaa

SEQ ID NO: 26 - protein, 1620 aa, Homo sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP